(19) United States
(12) Patent Application Publication
(10) Pub. No.: US 2006/0276436 A1
(43) Pub. Date: Dec. 7, 2006

(54) Farnesyl dibenzodiazepinone formulation

(75) Inventors: Maxime Ranger, Montreal (CA); Emmanuelle Roux, Montreal (CA)

Correspondence Address: ECOPIA BIOSCIENCES INC., 7290 FREDERICK-BANTING, SAINT-LAURENT, QC, H4S 2A1, CA

(73) Assignee: Ecopia BioSciences, Inc.

(21) Appl. No.: 11/443,186

(22) Filed: May 31, 2006

Related U.S. Application Data
(60) Provisional application No. 60/686,394, filed on Jun. 2, 2005.

(51) Int. Cl. A61K 31/675; A61K 31/551

(52) U.S. Cl. 514/80; 514/220

(57) ABSTRACT

The invention relates to pharmaceutical formulations comprising a farnesyl dibenzodiazepinone compound, or an analog, or a pharmaceutically acceptable salt or prodrug thereof, and a pharmaceutically acceptable surfactant and having improved chemical and biological properties. Such a formulation is a ready-to-use solution suitable for parenteral or non-parenteral administration or a bulk formulation for ex tempore preparation. The present invention also relates to therapeutic methods using the formulations, and methods for their preparation.

### RELATED APPLICATIONS

This application claims benefit under 35 USC §119 of Provisional Application U.S. Ser. No. 60/686,394, filed Jun. 2, 2005, the entire teachings of which are incorporated herein by reference for all purposes.

### FIELD OF THE INVENTION

The present invention relates to pharmaceutical formulations comprising a farnesyl dibenzodiazepinone compound, namely Compound 1 as defined below, or an analog, or a pharmaceutically acceptable salt or prodrug thereof. Such a formulation is a ready-to-use solution suitable for parenteral administration or non-parenteral administration, including oral or intranasal, or a bulk formulation for ex tempore reconstitution. Furthermore, the present invention also refers to methods of manufacture of formulations, to therapeutic methods of use of such formulations and their use in the manufacture of medicaments.

### BACKGROUND OF THE INVENTION

Compound 1, a novel farnesyl dibenzodiazepinone, was isolated from novel strains of actinomycetes, Micromonospora sp. Methods for the production of Compound 1 are disclosed in U.S. application Ser. No. 10/762,107 filed Jan. 21, 2004 and in WO 2004/065591 published in August 2004. Compound 1 also showed potent biological activities including anti-inflammatory, anti-bacterial and anti-cancer activity. U.S. application Ser. No. 10/951,436 filed Sep. 27, 2004 describes in vivo anti-cancer potency of the farnesyl dibenzodiazepinone Compound 1, in animal models. Analogs of Compound 1 are disclosed in U.S. Provisional Application 60/625,653 filed Nov. 8, 2004. Each of U.S. Ser. No. 10/762,107, WO 2004/065591, U.S. Ser. No. 10/951,436 and U.S. Ser. No. 60/625,653 are incorporated herein by reference in their entirety.

Farnesyl dibenzodiazepinones and analogs are lipophilic and not easily dissolved in aqueous media. In addition to enhanced solubility of the active compound, stability as well as physiological compatibility of the formulations is also required for parenteral administration.

One USP (United States Pharmacopeia) requirement for parenteral drug products is that the solution be visibly clear before use. Therefore, a vial of crystal clear solution is desired prior to administration, whether the solution is used directly or reconstituted from a powder or concentrate by the addition of solvent. Furthermore, to meet this standard, the number of particulates must be kept to a minimum. Particulates represent undissolved drug, which is ineffective and may block capillaries causing serious adverse health effects. However, formulations such as fat or lipid emulsions and suspensions have also been developed for parenteral use.

One method for the formulation of hydrophobic drugs is the use of surfactants. Among surfactant-using drug formulations marketed for pharmaceutical use in chemotherapy are VePesid™ (Etoposide with polysorbate 80), Vumon™ (Teniposide with Cremophor™ EL (polyoxyethylated castor oil)) and Taxol™ (Paclitaxel in Cremophor™ EL), all three from Bristol Myers Squibb, and Taxotere™ (Docetaxel in polysorbate 80) from Sanofi Aventis. Since the oral use of pharmaceutical surfactants is acceptable, bulk parenteral formulations using surfactants may also be used directly to produce oral preparations, such as gelatine capsules, gellules or incorporated in solutions, emulsions or suspensions.

Parenteral drug formulations are also prepared using liposome technology. Liposomal formulations are used, for example, to increase drug bioavailability, for tissue specific delivery, for the reduction of drug toxicity and to prevent precipitation, which can cause necrosis or other adverse effects at the site of injection. General principles of liposomal formulations for the delivery of chemotherapeutic agents were described in a review article published in 1999 (Drummond D. C. et al, Pharmacological Reviews (1999), vol. 51, no. 4, 691-743). Examples of liposomal drug formulations as successful pharmaceutical treatments are: antifungal agent amphotericin (Ambisome™, Gilead), and anticancer agents daunorubicin (DaunoXome™, Gilead) and doxorubicin (Doxil™, Alza, and Myocet™, Elan). Another example of liposomal formulation is the water insoluble benzoporphyrin which is marketed as Visudyne™ (QLT Phototherapeutics) for age-related macular degeneration.

Liposomal formulations for hydrophobic drugs have also been studied using Taxanes such as Paclitaxel and Docetaxel (Straubinger et al, Pharmaceutical Research (1994), vol. 11, no. 6, 889-896; Bernacki et al, Int. J. Cancer (1997), vol. 71, 103-107; Cattel et al, J. Control. Release (2000), vol. 63, 19-30 and (2003), vol. 91, 417429; Straubinger et al, AAPS PharmSci (2003), vol. 5, no. 4, Article 32, 1-11; Soepenberg et al, Eur. J. Cancer (2004), vol. 40, 681-688), using photosensitizers such as porphyrins (Reddi, J. Photochem. Photobio. B: Biology (1997), vol. 37, 189-195; Gurny et al, J. Photochem. Photobio. B: Biology (2002), vol. 66, 89-106; Sagristá et al, Int. J. Pharmaceutics (2004), vol. 278, 239-254; de Witte et al, Adv. Drug Delivery (2004), vol. 56, 17-30; Namiki et al, Pharmacological Research (2004), 65-76), and using thiazide diuretics such as HCT (hydrochlorothiazide) and CT (chlorothiazide) (Antimisiaris et al, J. Drug Targeting (2001), vol. 9, no. 1, 61-74).

### SUMMARY OF THE INVENTION

The present invention relates to suitable pharmaceutical formulations comprising a farnesyl dibenzodiazepinone compound as defined below, namely a compound of Formula I, any one of Compounds 1 to 130, Compound 1, any one of Compounds 2 to 7, 9 to 11, 14, 17, 18, 46, 63, 64, 67, 77, 78, 80, 82 to 85, 87, 89, 92, 95 to 98, 100 to 103, and 105, as defined below, or an ether, an ester, an N-alkylated or N-acylated derivative, or a pharmaceutically acceptable salt, solvate of prodrug of any one of the aforementioned compound as active ingredient, and a pharmaceutically acceptable carrier or vehicle.

In one aspect, the invention provides pharmaceutical formulations at a farnesyl dibenzodiazepinone concentration suitable for parenteral or nonparenteral delivery with or without mixing and/or dilution immediately prior to administration. In another embodiment, the formulation is a ready-to-use aqueous liquid solution suitable for parenteral administration. In another embodiment, the formulation is a bulk formulation for reconstitution immediately prior to parenteral administration. In a further embodiment, the formulation comprises a free, or liposomal farnesyl dibenzodiazepinone.

In one aspect, the invention provides a formulation comprising a farnesyl dibenzodiazepinone and a pharmaceutically acceptable hydrophobic carrier. In one embodiment, the hydrophobic carrier comprises at least one pharmaceutically acceptable surfactant. In another embodiment, the surfactant is a sorbitan ester, a phospholipid, tocopherol PEG succinate, or polyoxyethylated castor oil. In another embodiment, the surfactant is a sorbitan ester selected from polysorbate 80 (e.g. Tween™ 80 or Crillet 4 HP™), polysorbate 60, polysorbate 40 and polysorbate 20, more preferably a polysorbate 60 or 80, most preferably polysorbate 80. In another embodiment, the surfactant is polyoxyethylated castor oil. In another embodiment, the surfactant is a lipid, preferably a phospholipid or phospholipid derivative. In a subclass of this embodiment, when the surfactant is a phospholipid or phospholipid derivative, then the formulation is a liposomal formulation. Preferably liposomes diameter range from about 20 nm to about 1000 nm, more preferably about 80 nm to about 300 nm. In a further embodiment, the weight ratio of the surfactant to active ingredient is about 1:1 to about 100:1, preferably about 2:1 to about 50:1, more preferably about 5:1 to about 30:1, most preferably about 10:1 to about 25:1.

The invention further provides a formulation comprising a farnesyl dibenzodiazepinone or a pharmaceutically acceptable salt or prodrug thereof as active ingredient, a surfactant, and a pharmaceutically acceptable solvent. In one embodiment, the solvent is selected from ethanol, propylene glycol, glycofurol, N,N-dimethylacetamide, N-methylpyrrolidone and glycerin, preferably ethanol or propylene glycol, more preferably ethanol USP. In another embodiment, the formulation has a weight ratio of solvent to active ingredient, ranging from about 1:1 to about 100:1, preferably from about 1:1 to about 50:1, more preferably from about 1:1 to about 15:1, most preferably from about 2:1 to about 10:1.

The invention further provides a formulation comprising a farnesyl dibenzodiazepinone or a pharmaceutically acceptable salt or prodrug thereof as active ingredient, a surfactant and a solubilizer. In one embodiment, the formulation further comprises a solubilizer selected from cetrimide, docusate sodium, glyceryl monooleate, polyvinylpyrrolidone (Povidone, PVP) and poly(ethylene glycol) (PEG), preferably a hydrophilic polymer selected from PVP or PEG 400. In another embodiment, the weight ratio of solubilizer to active ingredient is about 1:1 to about 100:1, preferably from about 1:1 to about 50:1, more preferably from about 1:1 to about 15:1, most preferably from about 2:1 to about 10:1. In a further embodiment, the formulation further comprises a pharmaceutically acceptable solvent.

The invention further provides a formulation comprising a farnesyl dibenzodiazepinone or a pharmaceutically acceptable salt or prodrug thereof as active ingredient, a surfactant and an antioxidant. In one embodiment, the antioxidant is ascorbic acid or an ascorbate, such as sodium ascorbate. In another embodiment, the weight ratio of antioxidant to active ingredient is about 1:20 to about 20:1, preferably from about 1:10 to about 10:1, more preferably from about 1:5 to about 5:1, most preferably from about 1:5 to about 2:1. In a further embodiment, the invention further includes a pharmaceutically acceptable solvent or a solubilizer, or both.

The invention further provides a formulation comprising a farnesyl dibenzodiazepinone or a pharmaceutically acceptable salt or prodrug thereof as active ingredient, a surfactant and an aqueous medium. In one embodiment, the formulation is a bulk formulation and the aqueous medium is sterile water or water-for-injection. In another embodiment, the weight ratio of water to active ingredient is about 1:2 to about 50:1, preferably about 1:2 to about 25:1, more preferably about 1:1 to about 10:1, most preferably 1:1 to about 5:1. In another embodiment, the formulation is a ready-to-use solution and the aqueous media is water for injection, sterile water for injection, saline or dextrose in water, preferably 0.9% saline or 5% dextrose in water (D5W). In another embodiment, the concentration of active ingredient in the ready-to-use formulation is about 0.01 to about 50 mg/mL of the total volume of formulation, preferably about 0.05 to about 35 mg/mL, more preferably about 0.1 to about 20 mg/mL, most preferably about 1 to about 10 mg/mL. In a further embodiment, the formulation further comprises a pharmaceutically acceptable solvent, a solubilizer, or an antioxidant, or any combination thereof.

The invention also provides a method of preparing a bulk formulation, the method comprising the step of combining, with mixing, in any order, a farnesyl dibenzodiazepinone, or a pharmaceutically acceptable salt or prodrug thereof, and a surfactant. In one embodiment, the method comprises the incorporation of at least one solubilizer. In another embodiment, the method comprises the incorporation of at least one solubilizer selected from PVP and PEG 400. In another embodiment, the method comprises the incorporation of an additive, including a stabilizing agent, preferably an antioxidant. In a further embodiment, the antioxidant comprises at least one of ascorbic acid or ascorbate, preferably sodium ascorbate. In yet another embodiment, the additive comprises at least one of ascorbic acid or ascorbate, preferably sodium ascorbate, and an aqueous medium.

The invention further provides a method of preparing a formulation, the method comprising the steps of combining, with mixing: (a) the active ingredient and ethanol to obtain an ethanolic solution; (b) the antioxidant and sterile water to obtain an aqueous solution; (c) the hydrophilic polymer and the surfactant to obtain a mixture; (d) the ethanolic solution of step (a) and the mixture of step (c); and (e) the aqueous solution of step (b) and the solution of step (d) to produce the pharmaceutical formulation. In one embodiment, the formulation prepared is a bulk formulation.

In another aspect, the invention provides a method of preparing a ready-to-use formulation, the method comprising the steps of (a) providing a bulk formulation comprising a farnesyl dibenzodiazepinone in a form suitable for formulation, and (b) combining in any order, with mixing, the bulk formulation provided in (a) and an aqueous medium component. In one embodiment, the bulk formulation further comprises one or more additives. In a preferred embodiment, the additive is one or more solubilizers, from which one or more is preferably a surfactant. In another embodiment, optionally, the bulk farnesyl dibenzodiazepinone formulation is a liposomal form of a farnesyl dibenzodiazepinone or a pharmaceutically acceptable salt or prodrug thereof. In another embodiment, the aqueous medium is selected from water for injection, sterile water for injection, saline and dextrose in water, preferably 0.9% saline or 5% dextrose in water (D5W). In another embodiment, mixing step (b) is executed immediately prior to administration.

The invention further provides a method of preparing a ready-to-use formulation, the method comprising the steps of (a) providing a solid form comprising a farnesyl dibenzodiazepinone, and (b) combining in any order, with mixing, the solid form provided in (a) and a vehicle comprising a surfactant and an aqueous medium component and one or more additives. In one embodiment, the additive is selected from one or more solvent, one or more solubilizer or surfactant, and combinations thereof.

The invention further provides a method of preparing a formulation, the method comprising the steps of: (a) mixing in aqueous media a farnesyl dibenzodiazepinone with a lipid surfactant in such a manner that liposomes are formed, (b) lyophilizing the aqueous liposomal farnesyl dibenzodiazepinone to produce a bulk formulation. In one embodiment, the method further includes step (c) combining in any order, with mixing, the bulk formulation obtained in (b) and an aqueous media component to produce a ready-to-use formulation. In another embodiment, the bulk formulation comprises phospholipids. In another embodiment, the bulk formulation comprises phospholipids, and one or more additives. In another embodiment, the aqueous medium is selected from water for injection, sterile water for injection, saline and dextrose in water, preferably 0.9% saline or 5% dextrose in water (D5W). In another embodiment, mixing step (c) is executed immediately prior to administration.

In yet another aspect, the invention provides an article of manufacture, kit or commercial package, containing a parenterally deliverable pharmaceutical composition in a sealed vial and instructions for treatment of a neoplastic disorder. In one embodiment, the invention provides an article of manufacture comprising a first vial containing a bulk formulation of the invention and a second vial containing a physiologically suitable aqueous medium; wherein said aqueous medium in the second vial dissolves the bulk formulation in the first vial, and instructions for the treatment of a neoplastic disorder.

In a further aspect, this invention provides a commercial package, kit or system for continuous intravenous infusion, comprising a continuous intravenous infusion dosage of the compound of Formula I, or a pharmaceutically acceptable salt or prodrug thereof, together with instructions for use in the treatment of neoplasia in a mammal. In one embodiment, the infusion dosage is a concentrated form and the commercial package, kit or system further comprises a pre-filled syringe or other container containing an aqueous media for reconstitution of the infusion dosage. In another embodiment, the commercial package, kit or system further comprises an infusion bag. In another embodiment, the commercial package, kit or system further comprises connectors. In yet another embodiment, the commercial package, kit or system further comprises an administration set including a pump connector and anti-siphon valve. In another embodiment, the commercial package, kit or system further comprises an ambulatory infusion pump.

In another aspect, the invention provides an article of manufacture, kit or commercial package, containing a parenterally deliverable diluted or bulk formulation filled into a one or two compartment syringe to provide a ready-to-use product or ex tempore preparation product that will be used for parenteral administration.

The invention further provides an orally or intra-nasally deliverable formulation comprising a formulation as described above, and further comprising one or more additives. In one embodiment, the bulk formulation is filled into capsules, which are optionally enteric coated, and used for oral administration. In another embodiment, the bulk formulation is diluted into appropriate vehicle to form a solution, suspension or emulsion, and used for oral administration.

In a further aspect, the invention also provides a method of treating a subject having a condition or disorder wherein treatment with a farnesyl dibenzodiazepinone compound is indicated, namely tumor or neoplastic disorder, the method comprising the step of administering a therapeutically effective amount of a formulation as described herein. In one embodiment, the formulation is administered parenterally. The invention further provides the use of a formulation as described herein as an anti-tumor, anti-cancer or antineoplastic agent. The invention further provides the use of such a formulation in the manufacture of a medicament useful in the treatment of a neoplastic disorder.

Examples of neoplastic disorders, which may be treated by the formulations of the invention, include mammalian neoplasms such as leukemias, melanomas, central nervous system cancers (including glioblastoma, gliosarcoma, astrocytoma, and oligodendroglioma), breast cancers, lung cancers, pancreatic cancers, ovarian cancers, renal cancers, colon and colorectal cancers and prostate cancers. In another embodiment, the neoplastic disorder in the above-mentioned methods and uses, is selected from leukemia, breast cancer, prostate cancer, and CNS cancer.

### BRIEF DESCRIPTION OF THE DRAWINGS

FIG. 1: is a diagram showing the concentration of Compound 1, at 5 and 30 minutes after bolus injection of reconstituted Formulation B in different organs tissues and in plasma (plasma, liver, kidney, spleen, lung, fat and brain).

FIG. 2: shows the mean (±SD) plasma concentrations of Compound 1 in Swiss mice following 30 mg/kg intravenous (IV), intraperitoneal (IP), subcutaneous and (SC) bolus administration (using Formulation D11), and oral (PO) administration (using Formulation C).

FIG. 3: shows the mean concentration of Compound 1 in various tissues, 30 minutes after 30 mg/kg intravenous (IV), intraperitoneal (IP) and subcutaneous (SC) bolus administrations using Formulation D 1.

FIG. 4: shows in vivo antitumor activity of Compound 1 (Formulation D11) against the rat glioma (C6) tumor xenograft in female athymic (nu/nu) nude mice when given IP at 20 mg/kg (days 6-13) followed by 10 mg/kg (days 14-18) (upside down triangle), SC at 30 mg/kg (days 6-13) followed by 15 mg/kg (days 14-18) (square), and IV at 100 mg/kg (days 6-10 and days 13-17) (triangle), compared to the vehicle control group (circle) given IP at 5 mL/kg (days 6-18). Treatment was initiated when tumors were palpable (day 6).

FIG. 5: shows tumor volume growth curves of the different groups (mean±SEM) from in vivo antitumor activity of Compound 1 (Formulation D11) against the human glioma (U-87MG) tumor xenograft. Treatment was initiated when tumors were palpable (day 24). Compound 1 (30 mg/kg) (square) and drug-free control vehicle (5 mL/kg) (circle) were given SC once daily (Monday to Friday) for 2 weeks (q1d×5) 2 wk. Temodozolimide (diamond-shaped), used as positive control, was given PO at 150 mg/g every four days (total of 3 treatments).

FIG. 6: shows tumor volumes of all the animals from the different treatment groups of the in vivo activity assay of FIG. 5, when compared at day 34, after which time animals from the control group had to be sacrificed due to tumor burden.

FIG. 7: shows the antitumor efficacy of Compound 1 against human prostate tumor (PC3) xenografts in male Harlan nude mice, using Formulation D11 as bolus injections.

FIG. 8: shows the antitumor efficacy of Compound 1 against human prostate tumor (PC3) xenografts on individual male Harlan nude mice at day 22 of treatment, using bolus Formulation D11 administration.

FIG. 9: shows the antitumor efficacy of Compound 1 against human breast tumor (MDA-MB-231) xenografts in female Harlan nude mice, using Formulation D11 bolus administration.

FIG. 10: shows the antitumor efficacy of the compound of Formula I against human breast tumor (MDA-MB-231) xenografts on individual female Harlan nude mice at day 21 of treatment, using Formulation D11 bolus administration.

FIG. 11: shows the mean (±SD) plasma concentrations, during and post-infusion, of Compound 1 (Formulation D11) in Sprague-Dawley rats when administered continuous intravenous infusion (CIV) for 14 days (336 hours) at a dosage of 25 mg/kg/day, 50 mg/kg/day, and 75 mg/kg/day.

FIG. 12: shows the mean (±SD) plasma concentrations, during and post-infusion, of Compound 1 (Formulation D11) in Cynomolgus monkeys when administered CIV for 14 days (336 hours) at a dosage of 5 mg/kg/day, 15 mg/kg/day, and 30 mg/kg/day.

FIG. 13: shows a simulated Compound 1 plasma concentration-time profiles in humans, following a CIV infusion of Formulation D11 at 30 mg/m²/day for 14 days.

### DETAILED DESCRIPTION OF THE INVENTION

The invention relates to pharmaceutical formulations comprising a farnesyl dibenzodiazepinone or a pharmaceutically acceptable salt or prodrug thereof, and suitable for parenteral administration. In an embodiment of the invention, the formulation is a bulk composition comprising a farnesyl dibenzodiazepinone and a physiologically compatible vehicle, and optionally one or more additives. In another embodiment, the formulation is prepared immediately prior to parenteral administration.

The invention further relates to pharmaceutical formulations comprising a farnesyl dibenzodiazepinone or a pharmaceutically acceptable salt or prodrug thereof, loaded in liposomes and suitable for parenteral administration. In an embodiment of the invention, the formulation is a bulk composition comprising liposomal farnesyl dibenzodiazepinone and a physiologically compatible vehicle. In another embodiment, the formulation is prepared immediately prior to parenteral administration.

In yet another aspect, the invention provides methods for the preparation of said formulations. One method comprises the steps of providing a bulk farnesyl dibenzodiazepinone formulation and dissolving it in a pharmaceutically acceptable vehicle. In one aspect of the method, the bulk farnesyl dibenzodiazepinone formulation is a liposome preparation.

In an aspect, the invention provides methods of treating conditions such as tumor, pre-cancer and cancer conditions, said method comprising administering a formulation as described herein to a subject in need thereof.

In another aspect, the invention provides the use of a farnesyl dibenzodiazepinone formulation in the manufacture of a medicament for the treatment of said conditions. The invention further provides the use of a formulation of the invention in the treatment of a neoplastic disorder.

I. Definitions

Unless otherwise defined, all technical and scientific terms used herein have the meaning as commonly understood by a person skilled in the art to which this invention belongs.

The term “drug”, “active ingredient”, active pharmaceutical ingredient”, “API”, or “farnesyl dibenzodiazepinone” refers to a class of dibenzodiazepinone compounds containing a farnesyl moiety, and to derivatives of such compounds. The term includes, but is not limited to, 10-farnesyl-4,6,8-trihydroxy-dibenzodiazepin-11-one, which is referred to herein as Compound 1, or analogs of Compound 1, defined as Compounds 2 to 87 or the compounds of Formula I, or pharmaceutically acceptable salts or prodrugs thereof.

The term “pharmaceutically acceptable salt or prodrug” refers to any pharmaceutically acceptable ester, salt of an ester or any other derivative of a farnesyl dibenzodiazepinone, which upon administration to a mammal is capable of providing, either directly or indirectly, a compound of formula I or a biologically active metabolite or residue thereof. Particularly favored salts or prodrugs are those with improved properties, such as solubility, efficacy, or bioavailability of the compounds of this invention when such compounds are administered to the mammal (e.g., by allowing an orally administered compound to be more readily absorbed into the blood) or which enhance delivery of the parent compound to a biological compartment (e.g., the brain or lymphatic system) relative to the parent species. Pharmaceutically acceptable prodrugs of the compounds of this invention include, without limitation, carbamates, acyloxymethyl and acyloxyethyl derivatives, esters, amino acid esters, phosphate esters, sulfate and sulfonate esters. Salts refer to both acid addition salts and base addition salts. The nature of the salt is not critical, provided that it is pharmaceutically acceptable. Exemplary acid addition salts include, without limitation, hydrochloric, hydrobromic, hydroiodic, nitric, carbonic, sulphuric, phosphoric, formic, acetic, citric, tartaric, succinic, oxalic, malic, glutamic, propionic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric, galacturonic acid and the like. Suitable pharmaceutically acceptable base addition salts include, without limitation, metallic salts made from aluminium, calcium, lithium, magnesium, potassium, sodium and zinc or organic salts made from N,N′-dibenzylethylenediamine, chloroprocaine, choline, diethanolamine, ethylenediamine, N-methylglucamine, lysine, procaine and the like. Additional examples of pharmaceutically acceptable salts are listed in Berge et al, Journal of Pharmaceutical Sciences (1977), vol 66, no 1, 1-19. All of these salts may be prepared by conventional means from a farnesyl dibenzodiazepinone by treating the compound with the appropriate acid or base.

The term “solvate” refers to a physical association of a compound with one or more solvent molecules, whether organic or inorganic. This physical association includes hydrogen bonding. In certain instances the solvate will be capable of isolation, for example when one or more solvent molecules are incorporated in the crystal lattice of a crystalline solid. “Solvate” encompasses both solution-phase and isolable solvates. Exemplary solvates include hydrates, ethanolates, methanolates, and the like.

The term “formulation” or “composition” of the invention refers to ready-to-use pharmaceutically acceptable formulations, or to pharmaceutically acceptable reconstitutable bulk formulations comprising a farnesyl dibenzodiazepinone as defined below, and a pharmaceutically acceptable carrier or vehicle, suitable for parenteral administration or for oral or intranasal administration. As used herein, a “pharmaceutical composition” or “pharmaceutical formulation” comprises a pharmacologically effective amount of a farnesyl dibenzodiazepinone and a pharmaceutically acceptable carrier.

The term “bulk formulation” or “bulk composition” of the invention refers to pharmaceutically acceptable concentrated formulations, in bulk form, for later dispensing, formulation or compounding. The bulk formulation may be further formulated, or reconstituted to a form pharmaceutically acceptable for parenteral administration or oral or intranasal administration. The bulk formulation contains the active ingredient and a pharmaceutically acceptable carrier or vehicle. The bulk formulation optionally further comprises one or more additive and may optionally be a liposomal bulk formulation.

The terms “reconstituted” or “ready-to-use” formulation or composition of the invention, and equivalent expressions refer to pharmaceutically acceptable formulations having a ready-to-use concentration pharmaceutically acceptable for parenteral administration. The reconstituted formulation may be the result of the reconstitution or further dilution or production of a bulk formulation, to a form pharmaceutically acceptable and physiologically compatible for parenteral administration or oral or intranasal administration. The bulk formulation contains the active ingredient and a pharmaceutically acceptable carrier or vehicle. The bulk formulation optionally further comprises one or more additive and may optionally be a liposomal bulk formulation.

The term “pharmaceutically acceptable carrier” refers to one or more non-toxic, pharmaceutically-acceptable carriers and/or diluents and/or adjuvants and/or excipients, collectively referred to herein as “carrier” materials, and if desired includes other active ingredients or additives, for administration of a therapeutic agent. Examples of pharmaceutically acceptable carriers include, but are not limited to, solvents, vehicles or medium such as saline, buffered saline, 5% dextrose, water, glycerol, ethanol, propylene glycol, poly(ethylene glycol) (e.g. PEG 300 and 400), hydrophobic carriers, polysorbate 80 (e.g., Tween™ 80 or Crillet 4 HP™), polyoxyethylated castor oil (e.g. Cremophor EL™), poloxamer 407 and 188, and combinations thereof. The term specifically excludes cell culture medium. For drugs administered orally, examples of pharmaceutically acceptable carriers also include, but are not limited to pharmaceutically acceptable excipients such as inert diluents, disintegrating agents, binding agents, lubricating agents, sweetening agents, flavoring agents, coloring agents and preservatives.

The term “hydrophobic carriers” refers to carriers used for the pharmaceutical formulation of hydrophobic drugs. Examples of hydrophobic carriers include, without limitations, fat emulsions, surfactants, lipids, PEGylated phopholipids, polymer matrices, biocompatible polymers, and lipospheres, vesicles, micelles, particles and liposomes.

The terms “vehicle”, “solvent” or “medium” refers to a liquid that serves as solvent to dissolve the drug or formulation to obtain either a bulk formulation or ready-to-use formulation for parenteral administration. The vehicle may be aqueous, or water miscible (aqueous co-solvent), or non-aqueous (oleaginous). Examples of co-solvents include, without limitations, ethanol, propylene glycol, glycerine, poly(ethylene glycol) 300 NF, N-methylpyrrolidone, glycofurol, sorbitol and N,N-dimethylacetamide. Examples of aqueous vehicles or media include, without limitation, water for injection, 0.9% saline, buffered saine, and 5% dextrose in water (D5W). Examples of non-aqueous or oleaginous vehicles include, without limitation, peanut oil, corn oil, cottonseed oil, sesame oil, soybean oil, ethyl oleate, and isopropyl myristate.

The terms “pharmaceutically acceptable surfactant” or “surfactant” refer to a pharmaceutically acceptable substance, or a combination thereof, which reduces surface tension of a liquid, and lower the interfacial tension between two liquids. Surfactants are usually organic compounds that are amphipathic, meaning they contain both hydrophobic groups (their “tails”) and hydrophilic groups (their “heads”). Therefore, they are typically sparingly soluble in both organic solvents and water. A surfactant can be classified by the presence or absence of formally charged groups in its head. A nonionic surfactant has no charge groups in its head. The head of an ionic surfactant carries a net charge, if the charge is negative, the surfactant is anionic; if the charge is positive, it is cationic, if it contains a head with two oppositely charged groups, it is zwitterionic. Examples of surfactants include, without limitation, polyoxyethylated castor oil (e.g. Cremophor EL™), tocopherol PEG succinate, poloxamers (e.g. poloxamer 407 and 188), sorbitan esters such as polysorbate 80 (e.g. Tween™ 80 or Crillet 4 HP™), polysorbate 60, polysorbate 40 and polysorbate 20, and lipids (e.g. phospholipids). Further examples of surfactants suitable for pharmaceutical use are found, for example, in U.S. Pat. No. 6,761,903 (issued to Chen). Surfactants may also assemble in solution into aggregates that are known as micelles (e.g. polysorbates), or into liposomes (e.g. phospholipids).

The terms “liposome” and “liposomal formulation” refer to completely closed lipid bilayer membranes. Liposomes may be unilamellar vesicles (possessing a single bilayer membrane) or multilamellar vesicles (possessing multiple membrane layers, each separated from the next by an aqueous layer). The structure of the bilayer is such that the hydrophobic tails of the lipids orient toward the center and hydrophilic heads orient toward the aqueous phase. Examples of lipids used for the production of liposomes include, without limitation, natural or derived phospholipids, alpha tocopherol organic acid derivatives, and salt forms of cholesterol hemisuccinate, and combinations thereof. Phospholipids include, without limitation, phosphatidylcholines (e.g. EPC, HEPC, SPC, HSPC, DLPC, DMPC, DPPC, DSPC, DOPC, POPC), sphingomyelins (e.g. ESM, MSM), phosphatidylethanolamines (DMPE, DPPE, DSPE, DOPE), phosphatidylglycerols (e.g. EPG, DMPG, DPPG, DSPG, POPG), and phosphate (e.g. DMPA, DPPA, DSPA)), ceramides (e.g. C₂CER, C₈CER, C₁₄CER, C₁₆CER, C₁₈CER, C₂₀CER), and biotinated or pegylated phospholipids and ceramides (e.g. PEG₂₀₀₀DSPE, PEG₂₀₀₀DMPE, PEG₂₀₀₀DPPE and PEG₂₀₀₀CₙCER (n=8, 14, 20)). The liposomal formulation may also contain additives, such as cholesterol, which aid stabilization of the lipid bilayer. Other additives may also be employed, for example cryoprotectants or bulking agents (e.g. polyvinylpyrrolidone or mannitol), such as when the liposomal formulation is lyophilized to produce a bulk powder.

The term “liposomal drug” refers to a drug or active ingredient, which is isolated from the external aqueous phase by being included within the closed lipid bilayer membrane of the liposome, the drug may be present in the core of the vesicle or may be dissolved in the lipids of the lipid bilayer. Accordingly, the term “drug-loaded liposome” refers to the liposomal form including said active ingredient.

The terms “excipient” or “additive” refers to a pharmaceutically acceptable additive, other than the active ingredient, included in a formulation and having different purposes depending, for example on the nature of the drug, and the mode of administration. Examples of excipients include, without limitation: carriers, co-solvents, stabilizing agents, solubilizing agents and surfactants, buffers, antioxidants, tonicity agents, bulking agents, lubricating agents, emulsifiers, suspending or viscosity agents, antibacterial agents, chelating agents, preservatives, sweeteners, perfuming agents, flavouring agents, administration aids, and combinations thereof. Some of the excipients or additives may have more than one possible function or use, depending on their properties and the nature of the formulation.

The terms “solubilizing agent”, and “solubilizer” refer to a pharmaceutically acceptable excipient that enhances the solubility of the active ingredient in a physiologically acceptable formulation. Suitable solubilizing agents may include, without limitation, PVP (also known as polyvinylpyrrolidone or povidone) such as Kollidon™ 12 PF or 17PF, PEG (poly(ethylene glycol)) such as PEG 300 and 400 (e.g. Lutrol™ E400), cetrimide, docusate sodium, glyceryl monooleate, sodium lauryl sulfate, and surfactants.

The term “stabilizing agent” or “stabilizers” refers to a pharmaceutically acceptable excipient that enhances the physical or chemical stability of the active ingredient of the formulation. Examples of suitable stabilizing agents include, without limitation, buffers, antioxidants, chelating agents, cryo and lyoprotectants, delivery polymers (also solubilizers), bulking agents, tonicity agents and antibacterial agents.

The term “antioxidant” refers to a pharmaceutically acceptable excipient that prevents oxidation of the active ingredient by being oxidized faster than the active ingredient or by blocking oxidation. Examples of antioxidants include, without limitation, acetone sodium bisulfite, sodium bisulfite, butylated hydroxyanisole (BHA), butylated hydroxytoluene (BHT), cysteine, cysteinate hydrochloride, sodium dithionite, gentisic acid, gentisic acid ethanolamine, glutamic acid monosodium salt, sodium formaldehydesulfoxylate, potassium metabisulfite, sodium metabisulfite, monothioglycerol, propyl gallate, sodium sulfite, sodium thioglycolate, vitamin E, ascorbic acid and ascorbate salts, such as sodium ascorbate.

The term “emulsifier” or “emulsifying agent” refers to a pharmaceutically acceptable excipient that enhances the formation and stability of an emulsion, such as an oil or fat emulsion. Examples of emulsifiers include, without limitation, phospholipids, such as egg or soybean lecithin, or surfactants, such as poloxamers, and other polyoxyethylene derivatives such as polysorbates and polyoxyethylene castor oil.

The term “buffer” refers to a pharmaceutically acceptable excipient that helps to maintain the pH of the solution within a particular range specific to the buffering system, to prevent degradation and/or to keep adjusted to physiological pH. Suitable buffers include, without limitation, acetates, citrates, phosphates, tartrates, lactates, ascorbates, succinates, amino acids and the like.

The term “bulking agent” refers to a pharmaceutically acceptable excipient that adds bulk to a formulation which results in a well-formed cake upon drying, or freeze drying. Suitable bulking agents include, without limitation, mannitol, glycine, lactose, sucrose, trehalose, dextran, hydroxyethyl starch, ficoll and gelatin.

The term “tonicity agent” refers to a pharmaceutically acceptable excipient that, when added, reduces pain of injection by adjusting a hypotonic solution to isotonic so that the drug, when in solution, is physiologically compatible with the tissue cells of the patient. Examples of tonicity agents include, without limitation, glycerine, lactose, mannitol, dextrose, sodium chloride, sodium sulfate and sorbitol.

The term “antibacterial agent” refers to a pharmaceutically acceptable additive that prevents multiplication of microorganisms in a formulation. Examples of antibacterial agents include, without limitation, phenylmercuric nitrate, thimersol, benzethonium chloride, benzalkonium chloride, phenol, cresol, chlorobutanol.

The term “administration aid” refers to a pharmaceutically acceptable excipient that aids the administration, and/or activity of the drug. Examples of administration aids include, without limitation, local anesthetics (such as benzyl alcohol, xylocalne HCl and Procaine HCl), anti-inflammatory agents (such as hydrocortisone), anti-clotting agents (such as heparin), vaso-constrictor for prolongation (such as epinephrine), or agents that increase tissue permeability (such as hyaluronidase).

The term “v/v” refers to a concentration expressed in volume per total volume, of solution or mixture. For example, a percentage expressed in v/v refers to the number of millilitres of a constituent per 100 mL of solution or mixture.

The term “w/v” refers to a concentration expressed in weight per total volume of solution or mixture. For example, a percentage expressed in w/v refers to the number of grams of a constituent per 100 mL of solution or mixture.

The term “w/w” refers to a concentration expressed in weight per total weight of solution or mixture. For example, a percentage expressed in w/w refers to the number of grams of a constituent per 100 grams of solution or mixture.

As used herein, “weight ratio” refers to the amount of a first constituent compared to the amount of a second constituent, when both amounts are expressed by weight (e.g., in mg) and are both present in a formulation. For example, a formulation comprising a 1:5 weight ratio of active ingredient to surfactant will actually contain 5 mg of surfactant for each mg of active ingredient.

As used herein, “effective dose” means a dose that is deemed to be effective for a medical purpose (e.g. prophylactic or therapeutic) and will vary depending upon many factors. Such non-limiting factors include route and frequency of administration and medical purpose.

As used herein, the terms “unit dose” or “unit dosage” refer to physically discrete units suitable as unitary dosage for human subjects or other mammals, each unit containing a predetermined quantity of a farnesyl dibenzodiazepinone calculated to produce the desired therapeutic effect, in association with a suitable carrier. When a drug is administered over an extended period (e.g. via continuous intravenous infusion during 7 to 28 days), more than one discrete unit dose (e.g. ampoules or sealed vials) may be administered in a single administration event.

The term “reconstitution” refers to a process of returning a substance previously altered for preservation and storage to its original state, prior to administration, by addition of solvent or vehicle. For example, dilution of a concentrated liquid solution or suspension, or dissolution of a dry formulation, including dried or freeze-dried formulation.

The term “sterilization” refers to a process of substantially removing or neutralizing the microorganisms, which may be present with the drug after formulation, and/or before reconstitution of a bulk formulation, to prevent microbial proliferation and contamination of the patient. Examples of sterilization processes include, without limitation, steam sterilization, dry heat sterilization, filtration, gas sterilization, ionizing radiation.

The term “lyophilization” refers to a process of drying a drug or formulation solution; process in which water is sublimed from the product after it is frozen.

The terms “parenteral” and “parenteral administration” refer to bolus injection and/or infusion of a formulation in a para enteron mode of administration that is other than by the intestine, such as into or through the skin of a subject. Examples of parenteral modes of administration include, without limitation, intradermal, subcutaneous (s.c., s.q., sub-Q, Hypo), intramuscular (i.m.), intravenous (i.v.), intra-arterial, intramedulary, intracardiac, intra-articular (joint), intrasynovial (joint fluid area), intraspinal, intracranial and intrathecal (spinal fluids). Non-parenteral modes of administration include, without limitation, oral, intraocular, intranasal, topical, transdermal, rectal, sublingual and mucosal.

As used herein, abbreviations have their common meaning. Unless otherwise noted, the abbreviations “Ac”, “Me”, “Et”, “Pr”, “i-Pr”, “Bu”, and “Ph”, respectively refer to acetyl, methyl, ethyl, propyl(n- or iso-propyl), iso-propyl, butyl(n-, sec-, iso- or tert-butyl) and phenyl.

The term “alkyl” refers to linear, branched or cyclic, saturated hydrocarbon groups. Examples of alkyl groups include, without limitation, methyl, ethyl, n-propyl, isopropyl, n-butyl, pentyl, hexyl, heptyl, cyclopentyl, cyclohexyl, cyclohexylmethyl, and the like. Alkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, oxo, guanidino and formyl.

The term “C₁₋ₙalkyl”, wherein n is an integer from 2 to 12, refers to an alkyl group having from 1 to the indicated “n” number of carbons. The C₁₋ₙalkyl can be cyclic or a straight or branched chain.

The term “alkenyl” refers to linear, branched or cyclic unsaturated hydrocarbon groups containing, from one to six carbon-carbon double bonds. Examples of alkenyl groups include, without limitation, vinyl, 1-propene-2-yl, 1-butene-4-yl, 2-butene-4-yl, 1-pentene-5-yl and the like. Alkenyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidino. The double bond portion(s) of the unsaturated hydrocarbon chain may be either in the cis or trans configuration.

The term “C₂₋ₙalkenyl”, wherein n is an integer from 3 to 12, refers to an alkenyl group having from 2 to the indicated “n” number of carbons. The C₂₋ₙalkenyl can be cyclic or a straight or branched chain.

The term “alkynyl” refers to linear, branched or cyclic unsaturated hydrocarbon groups containing at least one carbon-carbon triple bond. Examples of alkynyl groups include, without limitation, ethynyl, 1-propyne-3-yl, 1-butyne-4-yl, 2-butyne-4-yl, 1-pentyne-5-yl and the like. Alkynyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl, formyl, oxo and guanidine.

The term “C₂₋ₙalkynyl”, wherein n is an integer from 3 to 12, refers to an alkynyl group having from 2 to the indicated “n” number of carbons. The C₂₋ₙalkynyl can be cyclic or a straight or branched chain.

The term “cycloalkyl” or “cycloalkyl ring” refers to an alkyl group, as defined above, further comprising a saturated or partially unsaturated carbocyclic ring in a single or fused carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. Cycloalkyl groups may optionally be substituted with substituents selected from acyl, amino, acylamino, acyloxy, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙcycloalkyl”, wherein n is an integer from 4 to 15, refers to a cycloalkyl ring or ring system or having from 3 to the indicated “n” number of carbons.

The term “heterocycloalkyl”, “heterocyclic” or “heterocycloalkyl ring” refers to a cycloalkyl group, as defined above, further comprising one to four hetero atoms (e.g. N, O, S, P) or hetero groups (e.g. NH, NR^{x}, PO₂, SO, SO₂) in a single or fused heterocyclic ring system having from three to fifteen ring members (e.g. tetrahydrofuranyl has five ring members, including one oxygen atom). Examples of a heterocycloalkyl, heterocyclic or heterocycloalkyl ring include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, and quinolizinyl. The foregoing heterocycloalkyl groups, as derived from the compounds listed above, may be C-attached or N-attached where such is possible. Heterocycloalkyl, heterocyclic or heterocycloalkyl ring may optionally be substituted with substituents selected from acyl, amino, acylamino, acyloxy, oxo, thiocarbonyl, imino, carboalkoxy, carboxy, carboxyamido, cyano, halo, hydroxyl, nitro, thio, alkyl, alkenyl, alkynyl, cycloalkyl, heterocycloalkyl, aryl, heteroaryl, alkoxy, aryloxy, sulfinyl, sulfonyl and formyl.

The term “C₃₋ₙheterocycloalkyl”, wherein n is an integer from 4 to 15, refers to an heterocycloalkyl group having from 3 to the indicated “n” number of atoms in the cycle and at least one hetero group as defined above.

The term “halo” refers to bromine, chlorine, fluorine or iodine substituents.

The term “aryl” or “aryl ring” refers to common aromatic groups having “4n+2” electrons, wherein n is an integer from 1 to 3, in a conjugated monocyclic or polycyclic system and having from five to fourteen ring atoms. Aryl may be directly attached, or connected via a C₁₋₃alkyl group (also referred to as aralkyl). Examples of aryl include, without limitation, phenyl, benzyl, phenethyl, 1-phenylethyl, tolyl, naphthyl, biphenyl, terphenyl, and the like. Aryl groups may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl.

The term “C₅₋ₙaryl”, wherein n is an integer from 5 to 14, refers to an aryl group having from 5 to the indicated “n” number of atoms, including carbon, nitrogen, oxygen and sulfur. The C₅₋ₙaryl can be mono or polycyclic.

The term “heteroaryl” or “heteroaryl ring” refers to an aryl ring, as defined above, further containing one to four heteroatoms selected from oxygen, nitrogen, sulphur or phosphorus. Examples of heteroaryl include, without limitation, pyridyl, imidazolyl, pyrimidinyl, pyrazolyl, triazolyl, tetrazolyl, furyl, thienyl, isoaxazolyl, thiazolyl, oxazolyl, isothiazolyl, pyrrollyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl groups. Heteroaryl may optionally be substituted with one or more substituent group selected from acyl, amino, acylamino, acyloxy, azido, alkythio, carboalkoxy, carboxy, carboxyamido, cyano, halo, hydroxyl, nitro, thio, alkyl, alkenyl, alkynyl, cycloalkyl, heterocyclyl, aryl, heteroaryl, alkoxy, aryloxy, sulfinyl, sulfonyl and formyl. Heteroaryl may be directly attached, or connected via a C₁₋₃alkyl group (also referred to as heteroaralkyl). The foregoing heteroaryl groups, as derived from the compounds listed above, may be C-attached or N-attached where such is possible.

The term “C₅₋ₙheteroaryl”, wherein n is an integer from 5 to 14, refers to an heteroaryl group having from 5 to the indicated “n” number of atoms, including carbon, nitrogen, oxygen and sulphur atoms. The C₅₋ₙheteroaryl can be mono or polycyclic.

The term “amino acid” refers to an organic acid containing an amino group. The term includes both naturally occurring and synthetic amino acids; therefore, the amino group can be but is not required to be, attached to the carbon next to the acid. A C-coupled amino acid substituent is attached to the heteroatom (nitrogen or oxygen) of the parent molecule via its carboxylic acid function. C-coupled amino acid forms an ester with the parent molecule when the heteroatom is oxygen, and an amide when the heteroatom is nitrogen. Examples of amino acids include, without limitation, alanine, valine, leucine, isoleucine, proline, phenylalanine, tryptophane, methionine, glycine, serine, threonine, cysteine, asparagine, glutamine, tyrosine, histidine, lysine, arginine, aspartic acid, glutamic acid, desmosine, ornithine, 2-aminobutyric acid, cyclohexylalanine, dimethylglycine, phenylglycine, norvaline, norleucine, hydroxylysine, allo-hydroxylysine, hydroxyproline, isodesmosine, allo-isoleucine, ethylglycine, beta-alanine, aminoadipic acid, aminobutyric acid, ethyl asparagine, and N-methyl amino acids. Amino acids can be pure L or D isomers or mixtures of L and D isomers.

II. Pharmaceutical Formulations and Methods for their Production

The invention relates to pharmaceutical formulations comprising a farnesyl dibenzodiazepinone, or a pharmaceutically acceptable salt or prodrug thereof, as active ingredient, and a pharmaceutically acceptable carrier or vehicle, as described below. Pharmaceutical formulations comprising a farnesyl dibenzodiazepinone are useful for treating a variety of diseases and disorders, particularly diseases associated with uncontrolled cellular growth and proliferation, such as neoplastic disorders. Farnesyl dibenzodiazepinones, or pharmaceutically acceptable salts or prodrugs thereof, are formulated and administered for the therapeutic or prophylactic treatment of diseases, particularly neoplastic disorders. The formulation comprises from about 0.1% to about 99.9%, about 1% to about 98%, about 5% to about 95%, about 10% to about 80% or about 15% to about 60% by weight of the active ingredient.

Active ingredients of interest for the novel formulation according to the present invention are farnesyl dibenzodiazepinones defined by Formula I: 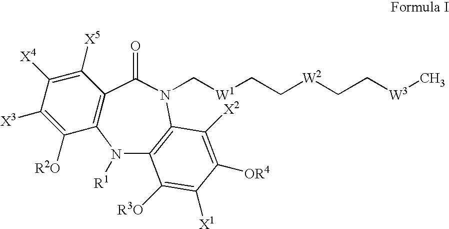
wherein,

W¹, W² and W³ are each independently selected from 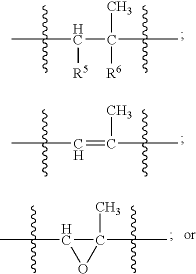
the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;

R¹ is selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;

R², R³, and R⁴ are each independently selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;

R⁵ and R⁶ are each independently selected from H, OH, OC₁₋₆alkyl, OC(O)C₁₋₆alkyl, NH₂, NHC₁₋₆alkyl, N(C₁₋₆alkyl)₂, NHC(O)C₁₋₆alkyl;

R⁷ is selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;

X¹, X², X³, X⁴ and X⁵ are each H; or

one of X¹, X², X³, X⁴ or X⁵ is halogen and the remaining ones are H; and

wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl;

and an ester, ether, N-alkylated or N-acylated derivative, or a pharmaceutically acceptable salt, solvate or prodrug thereof.

In one embodiment, R¹ is H, and all other groups are as previously disclosed. In another embodiment, R¹ is —CH₃, and all other groups are as previously disclosed. In another embodiment, R¹ is C₁₋₁₀alkyl, and all other groups are as previously disclosed. In a subclass of this embodiment, the alkyl group is optionally substituted with a substituent selected from halo, fluoro, C₆₋₁₀aryl, and C₅₋₁₀heteroaryl. In another embodiment, R¹ is —C(O)C₁₋₁₀alkyl, and all other groups are as previously disclosed. In another embodiment, R² is H, and all other groups are as previously disclosed. In another embodiment, R³ is H, and all other groups are as previously disclosed. In another embodiment, R⁴ is H, and all other groups are as previously disclosed. In another embodiment, R², R³ and R⁴ are each H, and all other groups are as previously disclosed. In another embodiment, one of R², R³ and R⁴ is CH₃, the others being each H, and all other groups are as previously disclosed. In another embodiment, two of R², R³ and R⁴ are CH₃, the other being H, and all other groups are as previously disclosed. In another embodiment, R², R³ and R⁴ are each CH₃, and all other groups are as previously disclosed. In another embodiment, R², R³ and R⁴ are each H, and W¹ is —CH═C(CH₃)—, and all other groups are as previously disclosed. In another embodiment, R², R³ and R⁴ are each H, and W² is —CH═C(CH₃)—, and all other groups are as previously disclosed. In another embodiment, R², R³ and R⁴ are each H, and W³ is —CH═C(CH₃)—, and all other groups are as previously disclosed. In another embodiment, R¹ is H and R², R³ and R⁴ are each H, and all other groups are as previously disclosed. In another embodiment, R¹ is H, each of W¹, W², and W³ is —CH═C(CH₃)—, and all other groups are as previously disclosed. In another embodiment, R¹ is H, each of W¹, W², and W³ is —CH₂CH(CH₃)—, and all other groups are as previously disclosed. In another embodiment, X¹ is Br, and each of X², X³, X⁴ and X⁵ are H, and all other groups are as previously disclosed. In another embodiment, if each of W¹, W² and W³ are —CH═C(CH₃)—, and each of R², R³, and R⁴ are H, then R¹ is not H. In a further embodiment, if each of W¹, W² and W³ are —CH═C(CH₃)—, and each of R², R³, and R⁴ are H, then R¹ is not CH₃. In a further embodiment, if each of W¹, W² and W³ are —CH═C(CH₃)—, and each of R², R³, and R⁴ are H, then R¹ is neither H nor CH₃. In a further embodiment, if the chain from the tricycle terminates at W¹ or W² with W² or W¹ respectively being either —CH═O, —CH(OC₁₋₆alkyl)₂, —CH₂OH, —CH₂OC₁₋₆alkyl or C(O)OR⁷, then R¹ is H. The invention encompasses all esters, ethers, N-alkylated or N-acylated derivatives, and pharmaceutically acceptable salts, solvates and prodrugs of the foregoing compounds.

Examples of specific interest are Compounds 1-130, defined as follows: 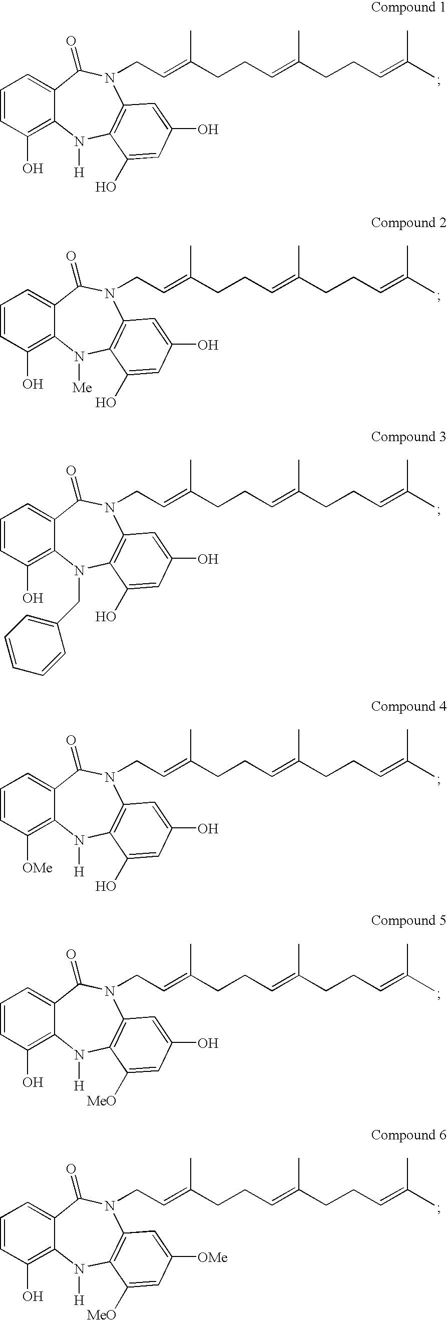 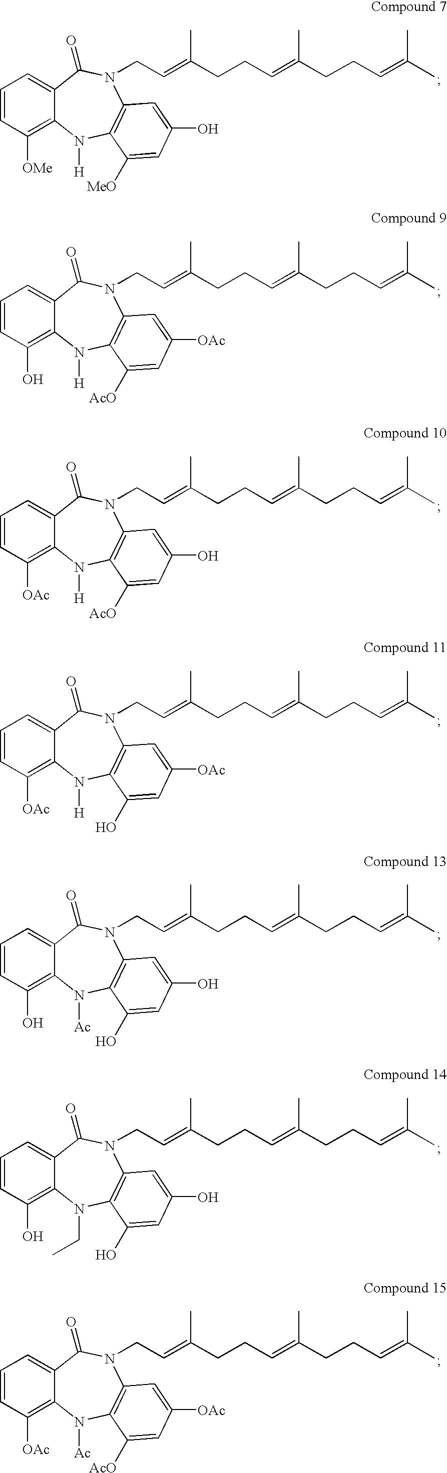 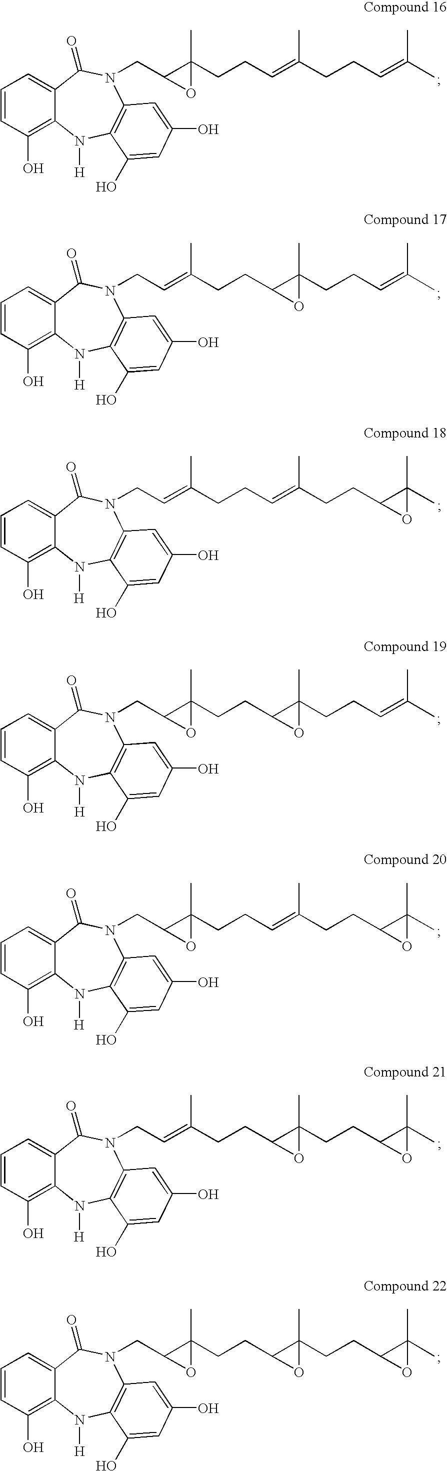 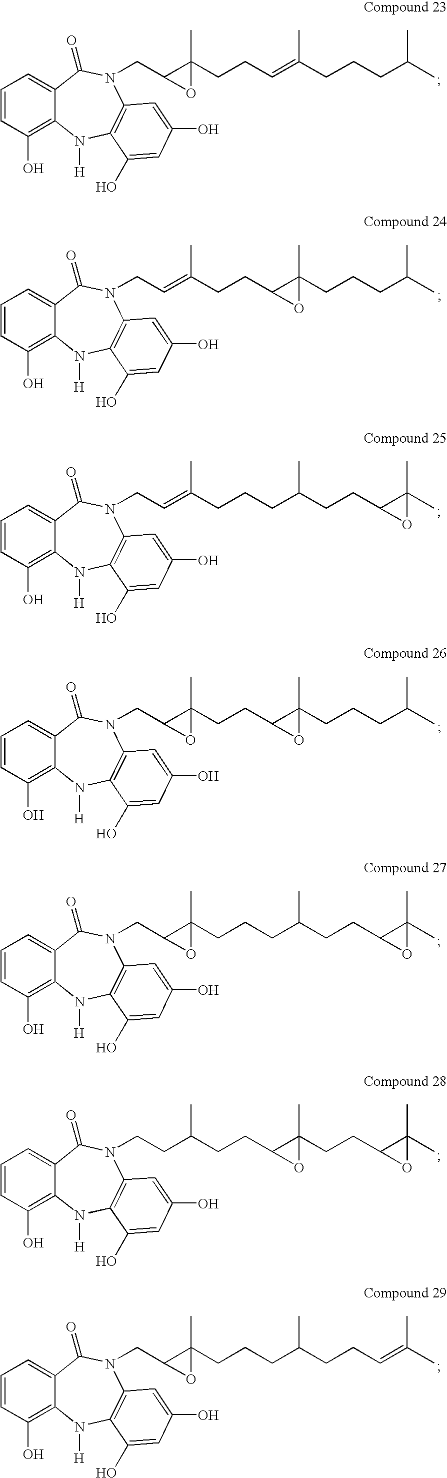 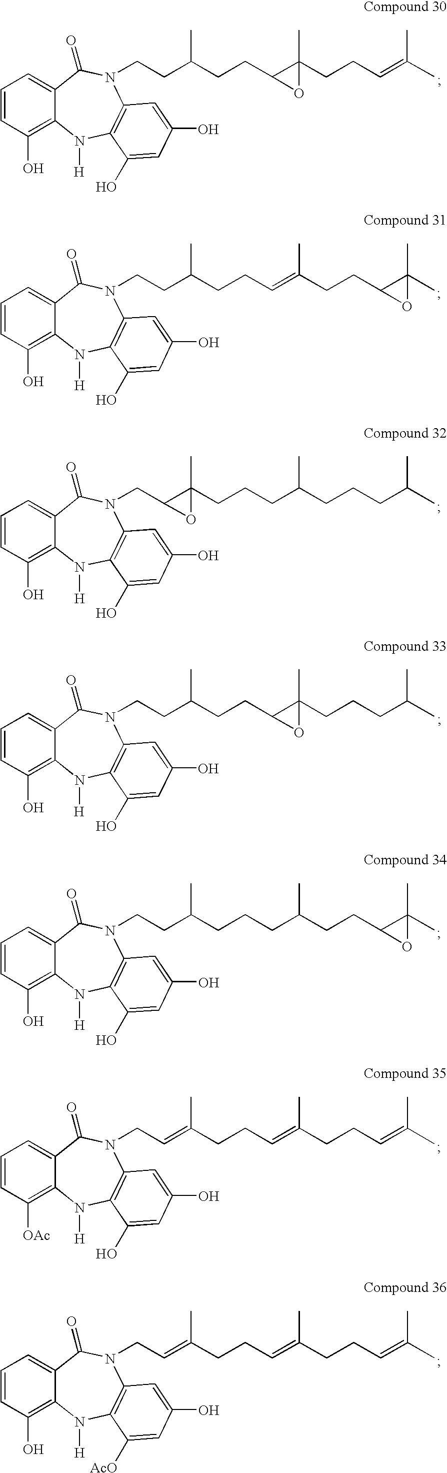 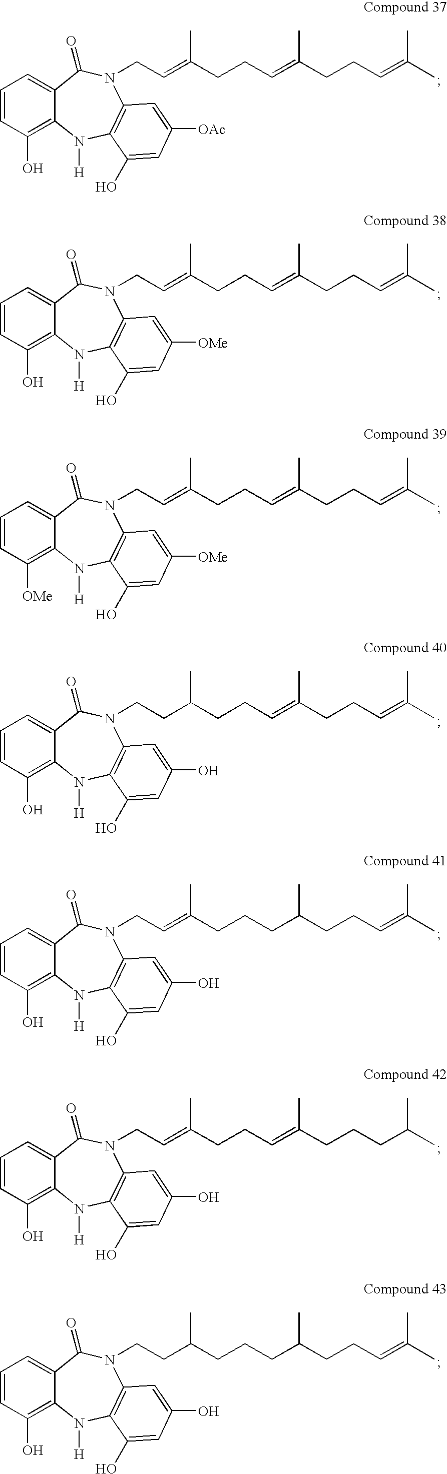 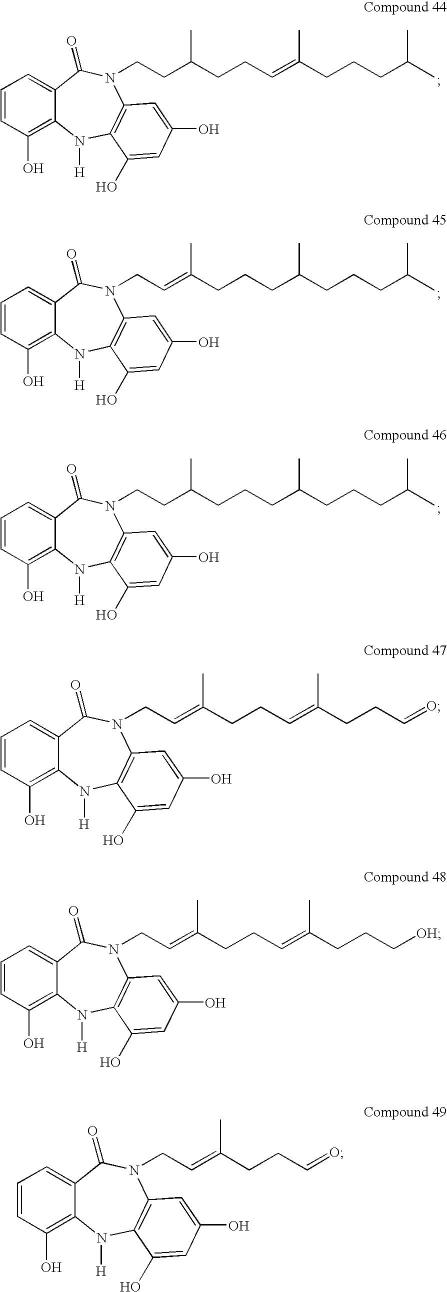 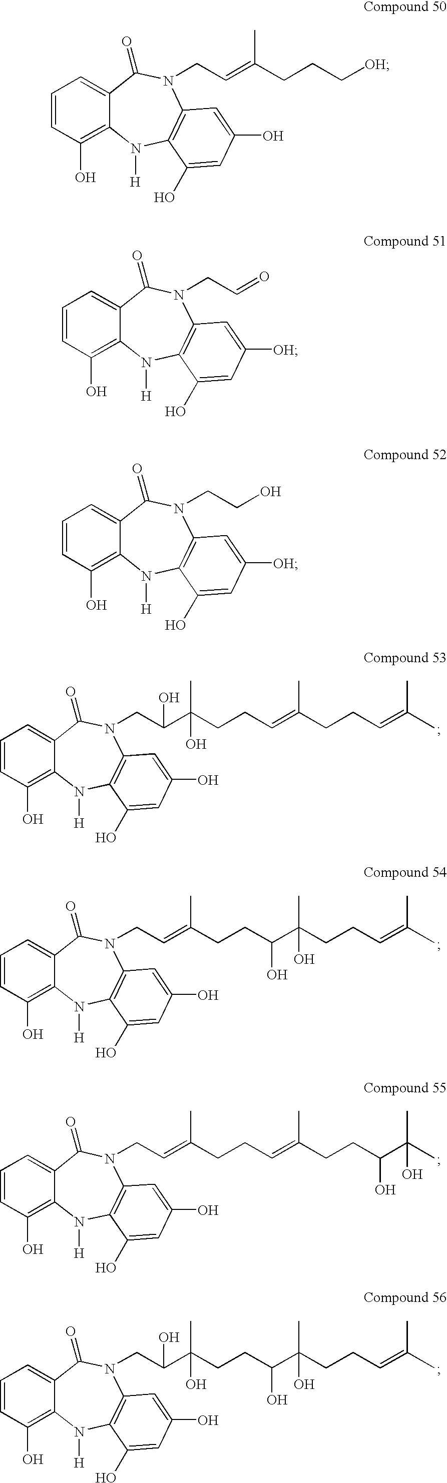 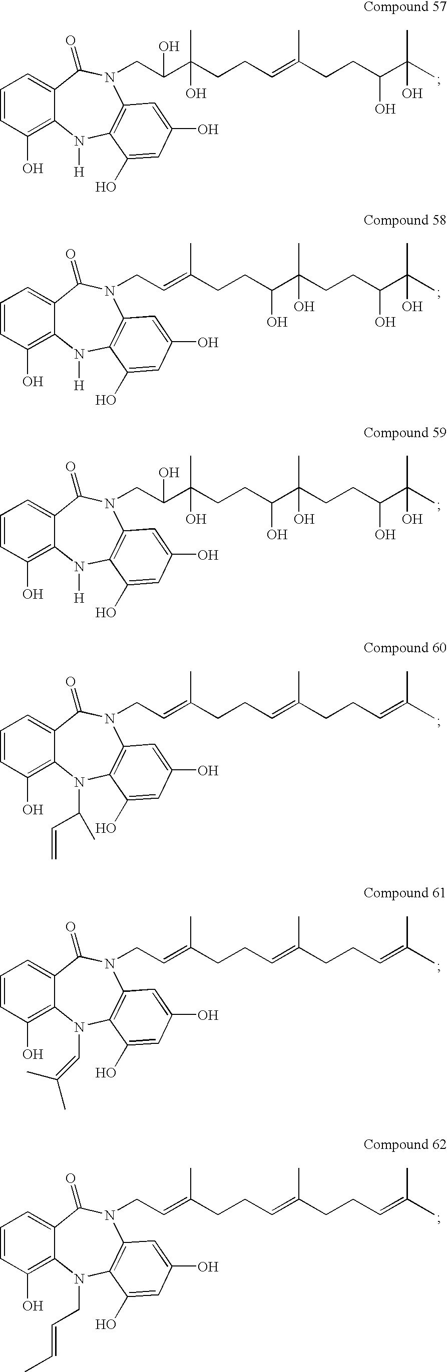 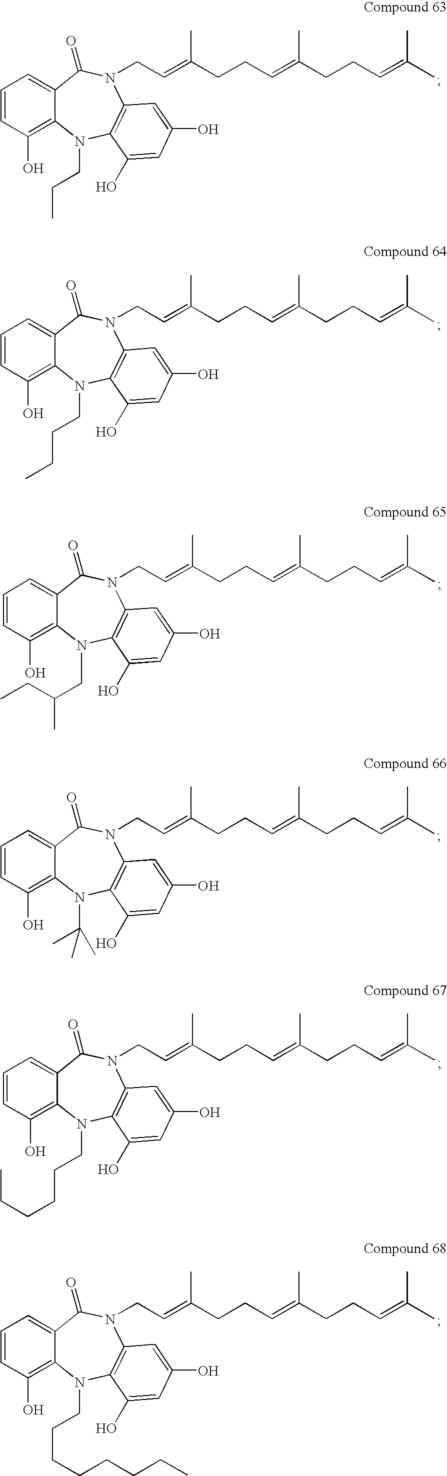 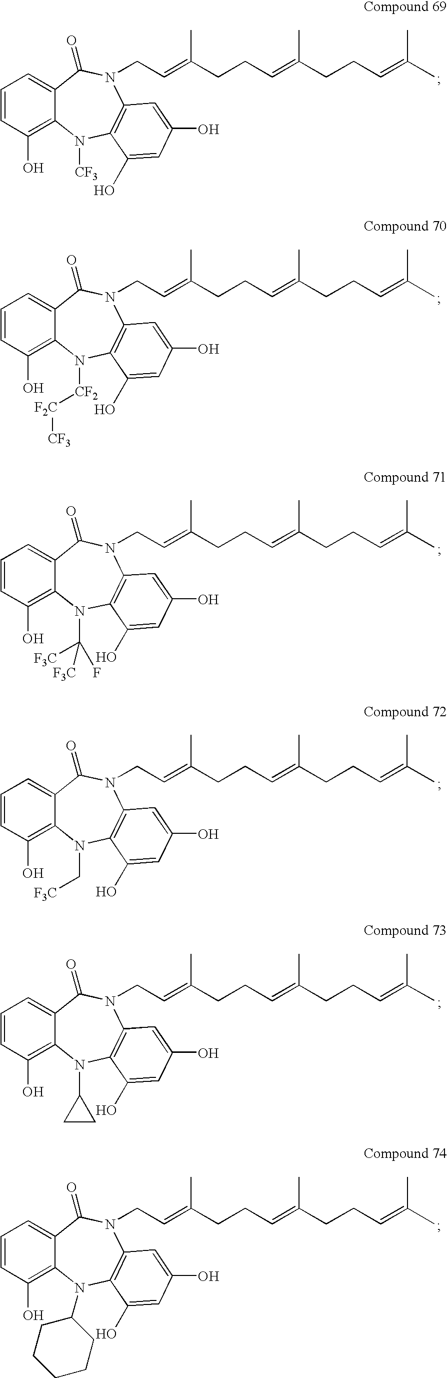 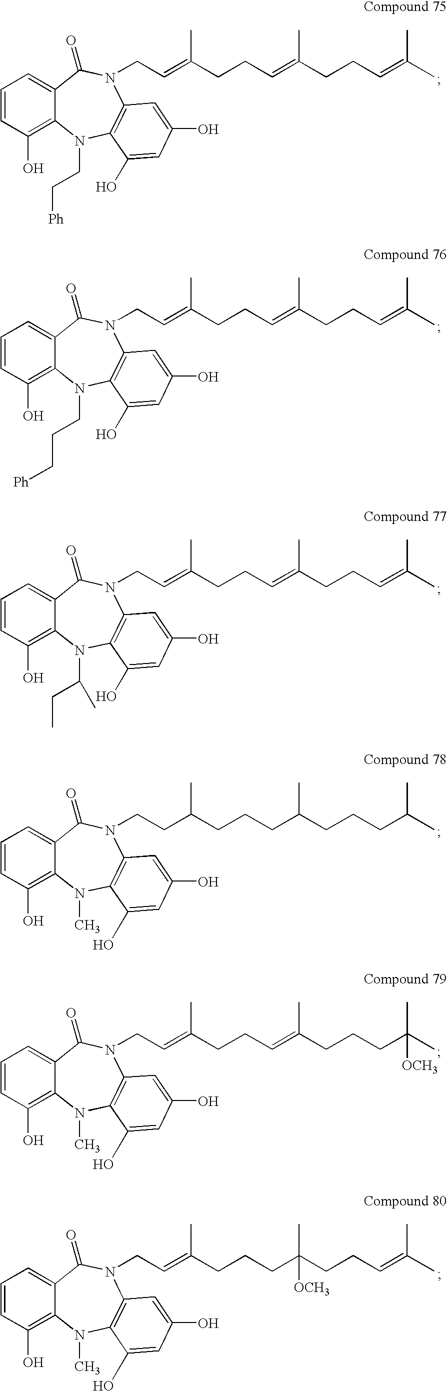 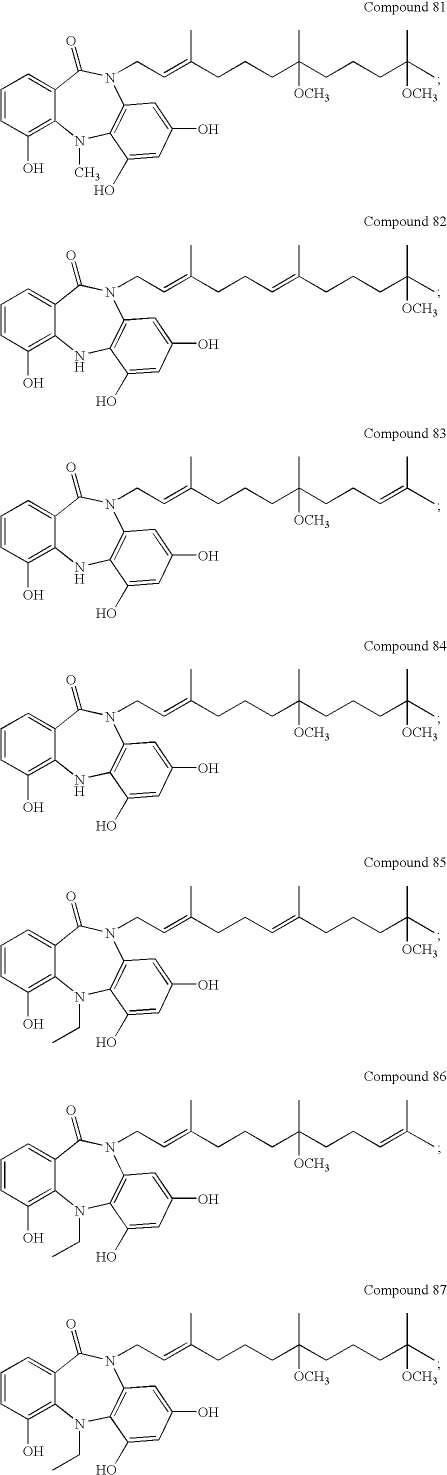 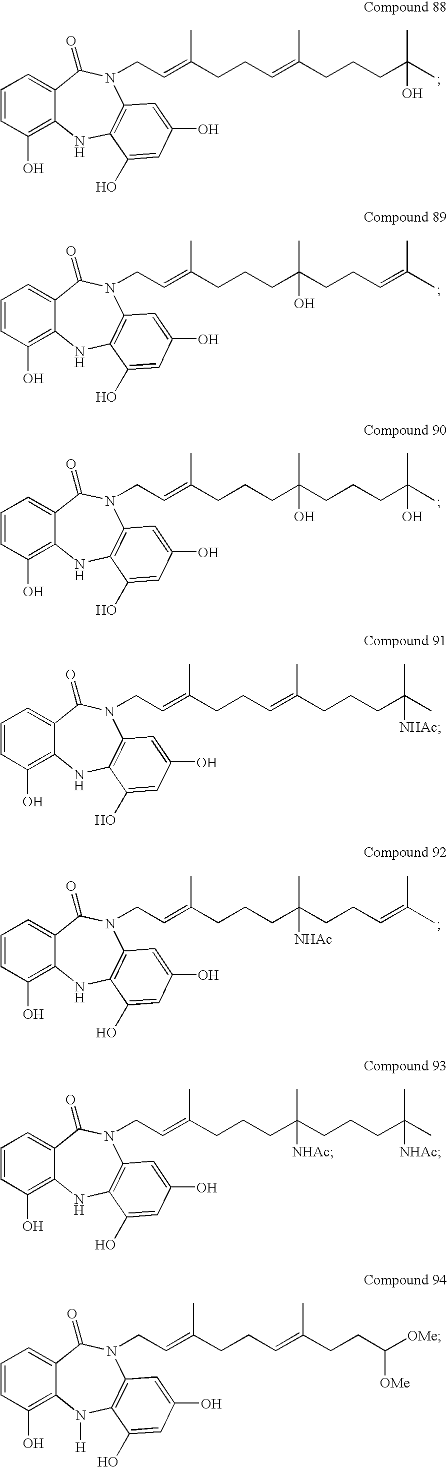 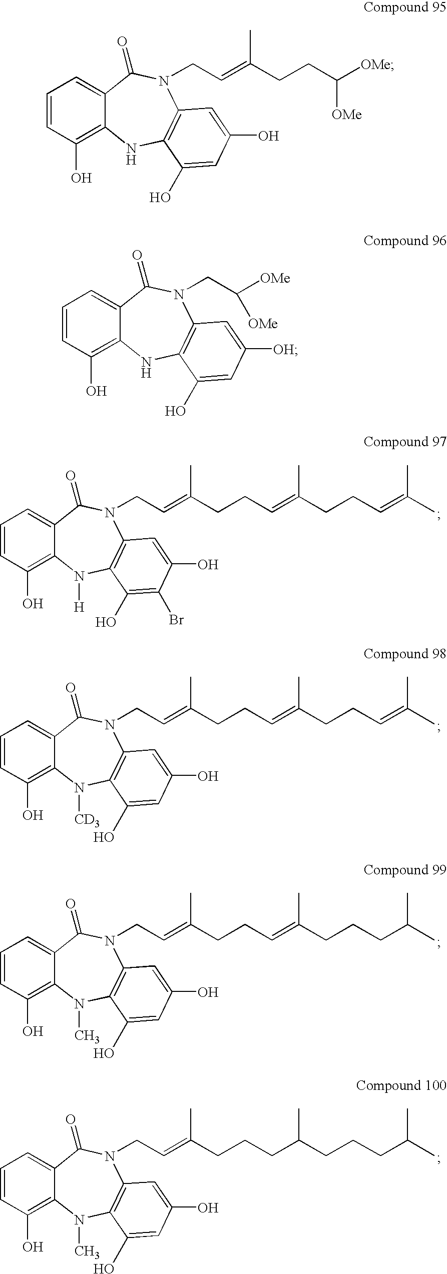 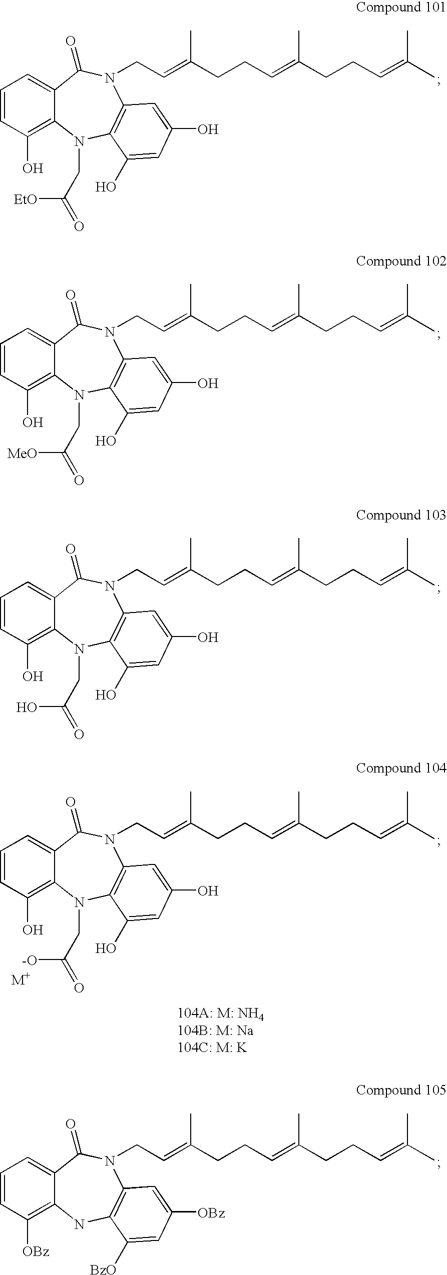 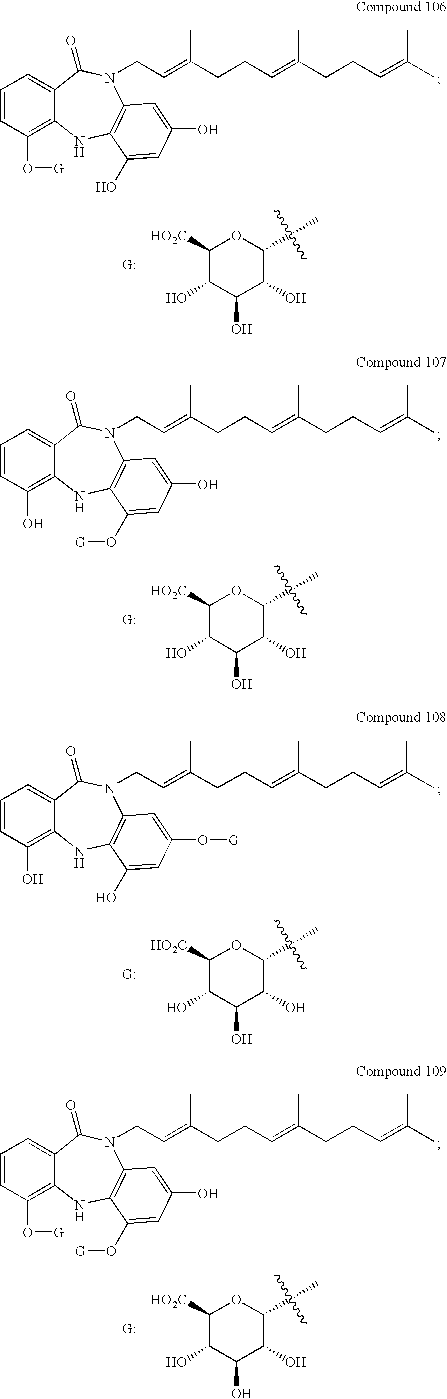 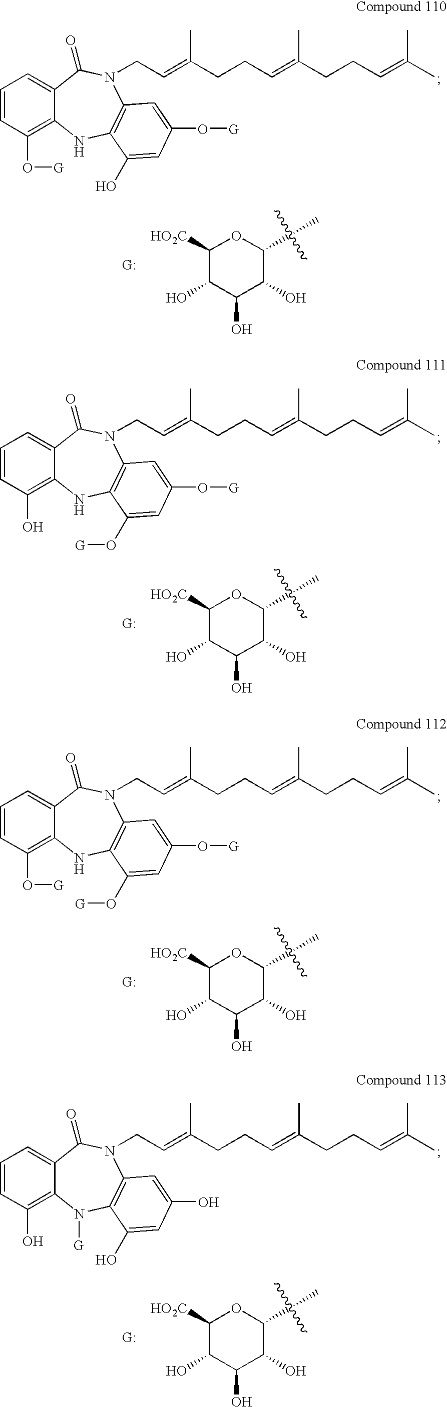 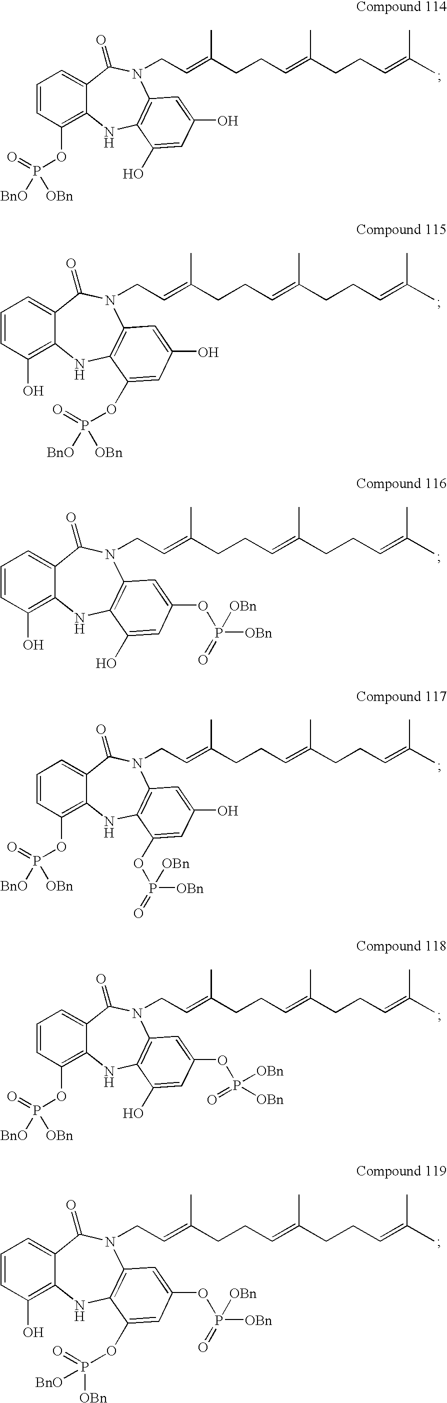 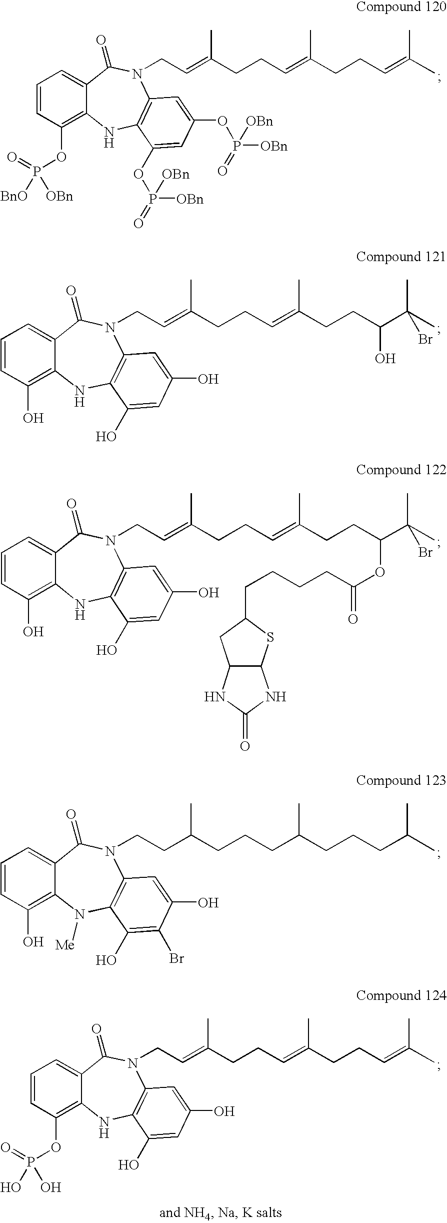 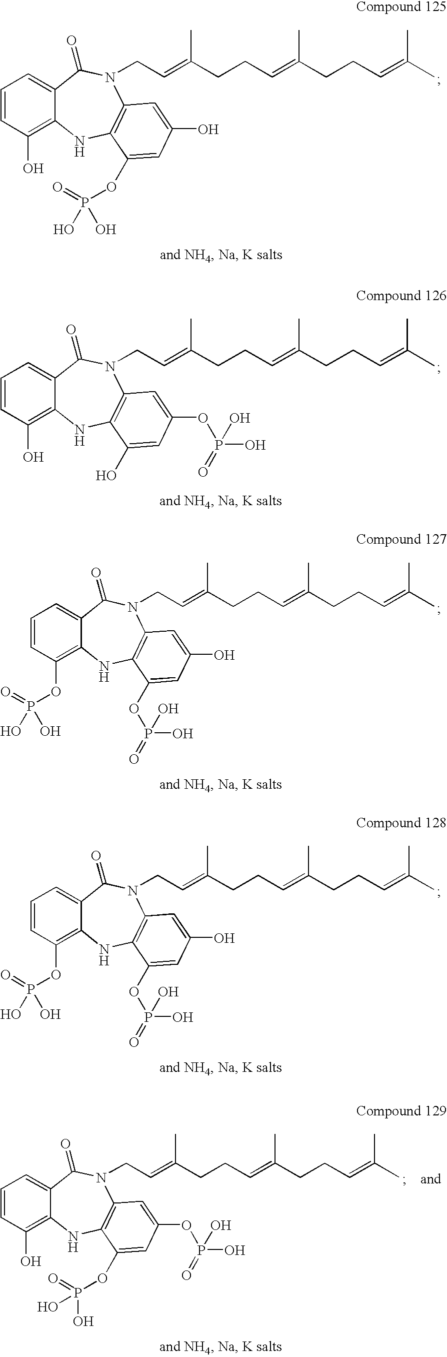 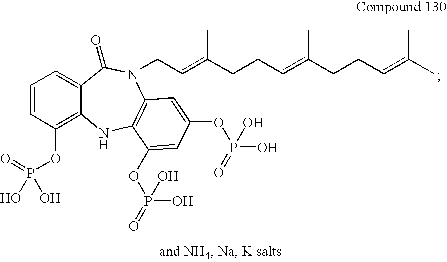
or a pharmaceutically acceptable salt, solvate or prodrug of any one of Compounds 1 to 130. Preferably the active ingredient is Compound 1, or a pharmaceutically acceptable salt, solvate or prodrug thereof.

The novel formulation according to the present invention comprises an active ingredient selected from farnesyl dibenzodiazepinones: Compound 1, a compound of Formula I, any one of Compounds 1-130, as defined above, or a pharmaceutically acceptable salt or prodrug thereof, together with a pharmaceutically acceptable carrier or vehicle, in a form suitable for parenteral or non-parenteral administration.

Pharmaceutically acceptable carriers refer to one or more non-toxic, pharmaceutically acceptable carriers and/or diluents and/or adjuvants and/or excipients, and/or vehicle, collectively referred to herein as “carrier” materials, for administration of a therapeutic agent. The carrier may optionally contain other active ingredients or additives. Pharmaceutically acceptable carriers and additives, other than the active ingredient, are included in a formulation and have different purposes depending, for example on the nature of the drug, and the mode of administration.

The compositions of the present invention can be delivered using controlled or sustained release delivery systems (e.g., bioerodable matrices). Exemplary delayed release delivery systems for drug delivery that are suitable for administration of the formulations of the invention (comprising a farnesyl dibenzodiazepinone) are described in U.S. Pat. No. 4,452,775 (issued to Kent), U.S. Pat. No. 5,039,660 (issued to Leonard), and U.S. Pat. No. 3,854,480 (issued to Zaffaroni).

A. Parenteral Pharmaceutical Formulations

Formulations for parental administration can be in the form of aqueous or non-aqueous isotonic sterile injection solutions, emulsions or suspensions, comprising a farnesyl dibenzodiazepinone, or a salt, solvate or prodrug thereof, as an active ingredient, and a pharmaceutically acceptable carrier. The parenteral form used for injection must be fluid to the extent that syringability exists and must be physiologically compatible. These solutions or suspensions are ready-to-use formulations suitable for parenteral administration or can be prepared from reconstitution of bulk formulations (e.g., concentrated liquids, powders or granules) immediately prior to administration.

Bulk formulations described herein are reconstituted prior to administration, in a pharmaceutically acceptable aqueous medium, such as water for injection, sterile water for injection, saline and dextrose in water, preferably 0.9% saline or 5% dextrose in water (D5W). In another embodiment, the concentration of active ingredient in the ready-to-use is about 0.01 to about 50 mg/mL of the total volume of formulation, preferably about 0.05 to about 35 mg/mL, more preferably about 0.1 to about 20 mg/mL, most preferably about 1 to about 10 mg/mL.

The parenteral formulations include a farnesyl dibenzodiazepinone and a pharmaceutically acceptable hydrophobic carrier including, for example, fat emulsions, and formulations containing surfactants, polymer matrices, biocompatible polymers, lipospheres, vesicles, micelles, particles, and liposomes. Fat emulsions include, in addition to the above-mentioned excipients, a lipid and an aqueous phase, and additives such as emulsifiers (e.g., phospholipids, poloxamers, polysorbates, and polyoxyethylene castor oil), and osmotic agents (e.g., sodium chloride, glycerol, sorbitol, xylitol, and glucose) to maintain the desired osmolarity.

The formulation may comprise one or more surfactant selected from a sorbitan ester, a lipid (e.g. phospholipids), tocopherol PEG succinate, poloxamer 407 and 188, or a polyoxyethylated castor oil (e.g., Cremophor EL™). Examples of sorbitan ester include polysorbate 80 (e.g., Tween™ 80 or Crillet 4 HP™), polysorbate 60, polysorbate 40 and polysorbate 20, preferably polysorbate 60 or 80, most preferably polysorbate 80. In one embodiment, the weight ratio of surfactant to active ingredient is about 1:1 to 100:1, preferably about 2:1 to 50:1, more preferably about 5:1 to 30:1, most preferably about 10:1 to about 25:1. Surfactants may form micelles, or liposomes, for example, where the surfactant is a lipid. Lipids may be selected from, for example, phospholipids and phospholipid derivatives such as phosphatidylcholine (PG), egg phosphatidylcholine (EPG), phosphatidylserine, phosphatidylglycerol, phosphatidylinositol, phosphatidic acid, dimyristoylphosphatidylcholine, dimyristoylphosphatidylglycerol and sphingomyelin. The liposome diameter may range from about 20 to about 1000 nm, preferably about 80 to about 300 nm. The formulation optionally comprises one or more additive, such as cholesterol, or cryoprotectants such as PVP or mannitol. The liposomal formulation is optionally lyophilized to produce a bulk formulation.

The bulk formulation may further comprise a pharmaceutically acceptable solvent. For example, the solvent may be selected from ethanol, corn oil, benzyl alcohol, propylene glycol, poly(ethylene glycol) 300 or 400 (PEG 300 and 400), glycofurol, N-methylpyrrolidone, sorbitol, N,N-dimethylacetamide, glycerin, preferably ethanol or propylene glycol, more preferably ethanol USP. The bulk formulation preferably has a weight ratio of solvent to active ingredient ranging from about 1:1 to about 100:1, from about 1:1 to about 50:1, from about 1:1 to about 15:1, or from about 2:1 to about 10:1 (wherein the density of ethanol at 25° C. is about 0.789 g/mL).

The formulation may further comprise one or more solubilizer including, for example, cetrimide, docusate sodium, glyceryl monooleate, polyvinylpyrrollidone (Povidone, PVP) and poly(ethylene glycol) (PEG), preferably a hydrophilic polymer such as PVP or PEG 400. The weight ratio of solubilizer to active ingredient is generally about 1:1 to about 100:1, about 1:1 to about 50:1, about 1:1 to about 15:1, or about 2:1 to about 10:1.

The formulation may further comprise additive(s), including one or more stabilizing agents, such as antioxidants. Preferred antioxidants include sodium ascorbate, with or without ascorbic acid. The weight ratio of antioxidant to active ingredient is generally about 1:20 to about 20:1, about 1:10 to about 10:1, or about 1:5 to about 5:1.

The bulk formulation may also include an aqueous media, preferably sterile water or water-for-injection, in a ratio of water to active ingredient of about 1:2 to about 50:1, about 1:2 to about 25:1, about 1:1 to about 10:1, or about 1:1 to about 5:1.

Bulk formulation may also be in a solid form (e.g. powder or granular) form for ex tempore reconstitution at the time of delivery. In addition to the above-mentioned excipients, solid forms optionally include bulking agents (e.g., mannitol, glycine, lactose, sucrose, trehalose, dextran, hydroxyethyl starch, ficoll, and gelatine), and cryo or lyoprotectants.

The pharmaceutical formulation may further contain administration aids, including local anaesthetics (such as benzyl alcohol, xylocalne HCl and Procaine HCl), anti-inflammatory agents (such as hydrocortisone), anti-clotting agents (such as heparin), vaso-constrictor for effect-prolongation (such as epinephrine), or agents that increase tissue permeability (such as hyaluronidase). These administration aids are used for patient comfort and/or drug delivery purposes.

The pharmaceutical formulation may also contain additives such as stabilizing agents including buffers, preservatives, antioxidants and antibacterial agents, and tonicity agents, which may serve to maintain the concentration of the active ingredient and the formulation into a physiologically acceptable form, a physiologically compatible sterile form, free of decomposition products, suspended particles and also free of microorganism contamination.

For example, in intravenous (IV) use (including continuous intravenous infusion), a sterile formulation of a compound of Formula I and one or more surfactants, can be dissolved or suspended in any of the commonly used intravenous fluids and administered by injection or infusion. Intravenous fluids include, without limitation, physiological saline, phosphate buffered saline, 5% glucose, or Ringer's™ solution. In intramuscular preparations, a sterile formulation of the compound of the present invention or suitable soluble salts or prodrugs forming the compound, can be dissolved and administered in a pharmaceutical diluent such as Water-for-Injection (WFI), physiological saline or 5% glucose. A suitable insoluble form of the compound may be prepared and administered as a suspension in an aqueous base or a pharmaceutically acceptable oil base, e.g. an ester of a long chain fatty acid such as ethyl oleate.

B. Non-Parenteral Pharmaceutical Formulations

Optionally, bulk parenteral formulations described above may be used directly to prepare a formulation for non-parenteral administration, for example for oral, topical or intranasal administration. One or more excipients or vehicle may be added to provide a more easily manipulated form. The bulk formulation described above, may be filled into gelatine capsules (optionally enteric coated), or used in suspensions or solutions, for oral administration.

For oral use, solid formulations such as tablets and capsules are particularly useful. Sustained release or enterically coated preparations may also be devised. For pediatric and geriatric applications, suspension, solutions and chewable tablets are especially suitable. For oral administration, the pharmaceutical compositions are in the form of, for example, tablets, chewable tablets, capsules, gelatine capsules, suspensions, emulsions, solutions or liquid syrups or elixirs, wafers and the like. For general oral administration, the formulation may contain one or more excipient or additives including, for example, inert diluents (e.g., sodium and calcium carbonate, sodium and calcium phosphate, and lactose), fillers (e.g., calcium phosphate, glycine, lactose, maize-starch, mannitol, sorbitol, or sucrose), disintegrating agents (e.g., potato starch, corn starch and alginic acid), binding agents (e.g., acacia gum, starch, gelatin, sucrose, polyvinylpyrrolidone (Povidone), sorbitol, or tragacanth methylcellulose, sodium carboxymethylcellulose, hydroxypropyl methylcellulose, and ethylcellulose), wetting agents, lubricating agents (e.g., magnesium stearate or other metallic stearates, stearic acid, poly(ethylene glycol), waxes, oils, silica and colloical silica, silicon fluid or talc), sweetening agents, perfuming agents, flavoring agents (e.g., peppermint, oil of wintergreen, fruit flavouring, cherry, grape, bubblegum, and the like), coloring agents and preservatives. Coloring agents may be used to make the dosage form more aesthetic in appearance or to help identify the product. The oral pharmaceutical composition is preferably made in the form of a unit dosage containing a therapeutically-effective amount of the active ingredient. Carriers may also include coating excipients such as glyceryl monostearate or glyceryl distearate, to delay absorption in the gastrointestinal tract.

Oral liquid preparations, generally in the form of aqueous or oily solutions, suspensions, emulsions, solutions or elixirs, may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, microcrystalline cellulose, methyl or propyl para-hydroxybenzoate, propylene glycol, sorbitol, or sorbic acid.

For topical use the compounds of present invention can also be prepared in suitable forms to be applied to the skin, or mucus membranes of the nose and throat, and can take the form of creams, ointments, nasal drop, liquid sprays or inhalants, lozenges, or throat paints. Such topical formulations further can include chemical compounds such as dimethylsulfoxide (DMSO) to facilitate surface penetration of the active ingredient. For application to the eyes or ears, the compounds of the present invention can be presented in liquid or semi-liquid form formulated in hydrophobic or hydrophilic bases as ointments, creams, lotions, paints or powders. For rectal administration the compounds of the present invention can be administered in the form of suppositories admixed with conventional carriers such as cocoa butter, wax or other glyceride.

Final concentration of active ingredient in the non-parenteral formulations (e.g., oral, topical or intranasal) may be higher than in parenteral formulations. The active ingredient may constitute from 10% to 100% by weight of the total formulation.

C. Methods of Manufacturing the Pharmaceutical Formulations

The formulations of the invention may be prepared according to any method known to the art of pharmaceutical manufacturing. Art recognized protocols and standards for the production of pharmaceutical formulations are available, for example in R. J. Strickley, Pharm. Res. (2004), vol. 21, no. 2, 201-230; M. J. Akers, J. Pharm. Sci. (2002), vol. 91, no. 11, 2283-2300 and B. Nuijen, Investigational New Drugs (2001), vol. 19,143-153.

The formulations are prepared according to FDA requirements and according to principles known to the art. The formulations of the invention are prepared and used at solvent and/or additive concentrations within acceptable ranges to produce a physiologically compatible reconstituted formulation. For example, the concentration in the ready-to-use formulation (reconstituted) of polysorbate 80 (e.g., Tween™ 80 or Crillet 4 HP™) is preferably less than 25% (v/v), PEG 400 is preferably less than 20% (v/v), PVP (e.g., Kollidon™ 12PF) is preferably less than 40% (v/v), and the concentration of ethanol is preferably less than 10% (v/v).

The method for preparing a ready-to-use formulation as described herein comprises the steps of (a) providing a bulk formulation comprising a farnesyl dibenzodiazepinone in a form suitable for formulation, and (b) combining in any order, with mixing, the bulk formulation provided in (a) and an aqueous medium component. Bulk and ready-to-use formulations are as described above. Preferably, mixing step (b) is executed immediately prior to administration.

The bulk formulation is provided by combining, with mixing, in any order, a farnesyl dibenzodiazepinone, or a pharmaceutically acceptable salt or prodrug thereof, a surfactant, optionally one or more solvents, optionally one or more solubilizers and optionally one or more stabilizers such as an antioxidant. Examples and ratios of surfactants, solvents, solubilizers and other excipients are provided above.

For example, a method of preparing the formulation comprises the steps of combining, with mixing: (a) the active ingredient and ethanol to obtain an ethanolic solution; (b) the antioxidant and sterile water to obtain an aqueous solution; (c) the hydrophilic polymer and the surfactant to obtain a mixture; (d) the ethanolic solution of step (a) and the mixture of step (c); and (e) the aqueous solution of step (b) and the solution of step (d) to produce the pharmaceutical formulation.

The invention further provides a method of preparing a formulation as described herein; the method comprising the steps of: (a) loading a farnesyl dibenzodiazepinone in liposomes in aqueous media, (b) lyophilizing the aqueous liposomal farnesyl dibenzodiazepinone to produce a bulk formulation, and (c) combining in any order, with mixing, the bulk formulation obtained in (b) and an aqueous media component. Preferably, the bulk formulation comprises a lipid surfactant, such as phospholipids, and optionally one or more additives. The aqueous medium is generally selected from water for injection, sterile water for injection, saline and dextrose in water, preferably 0.9% saline or 5% dextrose in water (D5W). Mixing step (c) may be executed immediately prior to parenteral administration. The formulation obtained from step (a) may be used directly for parenteral administration.

The farnesyl dibenzodiazepinone incorporation in liposomes is executed by conventional methods. Examples of procedures are found throughout the literature, and for example in: Straubinger et al, Pharmaceutical Research (1994), vol. 11, no. 6, 889-896; Bernacki et al, Int. J. Cancer (1997), vol. 71, 103-107; Cattel et al, J. Control Release (2003), vol. 91, 417-429; and Sagristá et al, Int. J. Pharmaceutics (2004), vol. 278, 239-254. As an exemplary procedure, phospholipids and the active compound (2-25 mol % vs lipids, preferably 4-20 mol %) are dissolved in an organic solvent such as methanol, chloroform, dichloromethane, tetrahydrofuran, or a combination thereof, and optionally comprising a vesicle stabilizing cholesterol agent. The organic solvent is removed in vacuo and/or by nitrogen stream. The lipids-active ingredient complex is swelled in a vehicle or aqueous media and optionally passed through an extruder to homogenize vesicles sizes. The liposomal formulation may optionally be lyophilized and reconstituted prior administration, or may be diluted directly with aqueous media suitable for parenteral administration.

Pharmaceutically acceptable salt of a farnesyl dibenzodiazepinone, or a prodrug thereof may be generated in situ in the vehicle by adding the corresponding acid or base, or prior to formulation.

The formulation, bulk or reconstituted, may be sterilized using any art-recognized technique. Preferably, the formulation is sterilized by filtration before or after reconstitution.

The formulations of the invention may be hermetically sealed in ampoules, vials or containers until use. The container may be capped under sterile environment with a stopper made of rubber or other polymeric material, optionally coated with Teflon™ (polytetrafluoroethylene). The vial or ampoule may contain one unit dose of the farnesyl dibenzodiazepinone formulation of this invention. A unit dose is an amount of a formulation comprising an amount of a farnesyl dibenzodiazepinone, such amount being suitable for delivery in a single administration event. However, more than one discrete unit dose (e.g. ampoule or sealed vial) may be used when the formulation is administered over an extended period of time, e.g. by continuous intravenous infusion. A unit dose of a formulation having a farnesyl dibenzodiazepinone as active ingredient may contain about 10 to 3000 mg of active ingredient, or about 20 to 1000 mg of active ingredient. The hermetically sealed unit dosage formulation can be a ready-to-use formulation of the compound or a salt or prodrug thereof in a suitable vehicle. Optionally, the formulation may also be filled in a syringe as a ready-to-use.

The hermetically sealed container may also contain a unit dose of a bulk formulation. A second container or vial containing a suitable sterile solvent or vehicle may also be provided, along with instructions on how to dissolve the content of the first container prior to administration, preferably the vehicle is an aqueous media. The bulk formulation may also be filled into a one- or two-compartment syringe to provide a preparation product that will be used for parenteral administration after reconstitution in the appropriate sterile vehicle.

The pharmaceutical formulation may be packaged into a convenient commercial package providing the necessary material, such as the pharmaceutical formulation as described herein, and written instructions for its use in treating a neoplastic condition, in a suitable container.

III. Modes of Administration and Methods of Treating Neoplastic Disorders

The pharmaceutical formulations disclosed herein are prepared in accordance with standard procedures (USP, FDA) and are administered at dosages that are selected to reduce, prevent, or eliminate neoplastic cells, neoplasms, cancers or pre-cancers. (See, e.g., Remington's Pharmaceutical Sciences, Mack Publishing Company, Easton, Pa.; and Goodman and Gilman, Pharmaceutical Basis of Therapeutics, Pergamon Press, New York, N.Y., the contents of which are incorporated herein by reference, for a general description of the methods for administering various medicaments for human therapy, including chemotherapy). The pharmaceutical formulations of this invention may be administered parenterally or by non-parenteral routes, such as oral, topical or intranasal. Parenteral routes of administration include intradermal, subcutaneous (SC, s.q., sub-Q, Hypo), intramuscular (IM), intravenous (IV) and continuous intravenous infusion (CIV), intra-arterial, intramedulary, intracardiac, intra-articular (joint), intrasynovial point fluid area), intracranial, intraspinal and intrathecal (spinal fluids). Any known device useful for parenteral injection or infusion of drug formulations can be used to effect such administration.

The invention relates to a method for inhibiting growth and/or proliferation of cancer cells in a mammal and a method of treating a neoplastic condition in a mammal. Mammals include ungulates (e.g. sheeps, goats, cows, horses, pigs), and non-ungulates, including rodents, felines, canines and primates (i.e. human and non-human primates). Preferably, the mammal is a human.

As used herein, the terms “neoplasm”, “neoplastic disorder”, “neoplasia” “cancer,” “tumor” and “proliferative disorder” refer to cells having the capacity for autonomous growth, i.e., an abnormal state of condition characterized by rapidly proliferating cell growth which generally forms a distinct mass that show partial or total lack of structural organization and functional coordination with normal tissue. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or maligriantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, esophagus cancers, colon cancers, colorectal cancers, gastro-intestinal cancers, melanomas, ovarian and endometrial cancer, renal and bladder cancer, liver cancer, endocrine cancer (e.g. thyroid), and pancreatic cancer.

The farnesyl dibenzodiazepinone is brought into contact with or introduced into a cancerous cell or tissue. In general, the methods of the invention for delivering the pharmaceutical compositions of the invention in vivo utilize art-recognized protocols for delivering therapeutic agents with the only substantial procedural modification being the substitution of the farnesyl dibenzodiazepinone of the present invention for the therapeutic agent in the art-recognized protocols. The route by which the farnesyl dibenzodiazepinone-containing formulation is administered, as well as the formulation, carrier or vehicle will depend on the location as well as the type of the neoplasm. A wide variety of administration routes can be employed. The farnesyl dibenzodiazepinone formulation may be administered by intravenous or intraperitoneal infusion or injection. For example, for a solid tumor or neoplasm that is accessible, the formulation may be administered by injection directly into the tumor or neoplasm. For a hematopoietic neoplasm the formulation may be administered intravenously or intravascularly. For neoplasms that are not easily accessible within the body, such as metastases or brain tumors, the formulation may be administered in a manner such that it can be transported systemically through the body of the mammal and thereby reach the neoplasm and distant metastases for example intrathecally, intravenously or intramuscularly or orally. The farnesyl dibenzodiazepinone-containing formulation can also be administered subcutaneously, intraperitoneally, topically (for example for melanoma), rectally (for example colorectal neoplasm), vaginally (for example for cervical or vaginal neoplasm), nasally or by inhalation spray (for example for lung neoplasm).

The farnesyl dibenzodiazepinone formulation is administered in an amount that is sufficient to inhibit the growth or proliferation of a neoplastic cell, or to treat a neoplastic disorder. The term “inhibition” refers to suppression, killing, stasis, or destruction of cancer cells. The inhibition of mammalian cancer cell growth according to this method can be monitored in several ways. Cancer cells grown in vitro can be treated with the compound and monitored for growth or death relative to the same cells cultured in the absence of the compound. A cessation of growth or a slowing of the growth rate (i.e., the doubling rate), e.g., by 50% or more at 100 micromolar, is indicative of cancer cell inhibition (see Anticancer Drug Development Guide: preclinical screening, clinical trials and approval; B. A. Teicher and P. A. Andrews, ed., 2004, Humana Press, Totowa, N.J.). Alternatively, cancer cell inhibition can be monitored by administering the pharmaceutical formulation to an animal model of the cancer of interest. Examples of experimental non-human animal cancer models are known in the art and described below and in the examples herein. A cessation of tumor growth (i.e., no further increase in size) or a reduction in tumor size (i.e., reduction of tumor volume by least a 58%) in animals treated with the formulation relative to tumors in control animals not treated with the formulation is indicative of significant tumor growth inhibition (see Anticancer Drug Development Guide: preclinical screening, clinical trials and approval; B. A. Teicher and P. A. Andrews, ed., 2004, Humana Press, Totowa, N.J.).

The term “treatment” refers to the application or administration of a farnesyl dibenzodiazepinone-containing formulation to a mammal, or application or administration of a formulation to an isolated tissue or cell line from a mammal, who has a neoplastic disorder, a symptom of a neoplastic disorder or a predisposition toward a neoplastic disorder, with the purpose to cure, heal, alleviate, relieve, alter, ameliorate, improve, or control the disorder, the symptoms of disorder, or the predisposition toward disorder. The term “treating” is defined as administering, to a mammal, an amount of a farnesyl dibenzodiazepinone-containing formulation sufficient to result in the prevention, reduction or elimination of neoplastic cells in a mammal (“therapeutically effective amount”). The therapeutically effective amount and timing of dosage will be determined on an individual basis and may be based, at least in part, on consideration of the age, body weight, sex, diet and general health of the recipient subject, on the nature and severity of the disease condition, and on previous treatments and other diseases present. Other factors also include the route and frequency of administration, the activity of the administered compound, the metabolic stability, length of action and excretion of the compound, drug combination, the tolerance of the recipient subject to the compound and the type of neoplasm or proliferative disorder. In one embodiment, a therapeutically effective amount of the compound is in the range of about 0.5 mg/kg to about 750 mg/kg of body weight of the mammal, per day. In another embodiment, the therapeutically effective amount is in the range of about 0.5 mg/kg to about 300 mg/kg body weight per day. In yet another embodiment, the therapeutically effective amount is in the range of 1 mg/kg to about 50 mg/kg body weight per day. The therapeutically effective doses of the above embodiments may also be expressed in milligrams per square meter (mg/m²) of body surface, for example in the case of human patients. Conversion factors for different mammalian species may be found in: Freireich et al, Quantitative comparison of toxicity of anticancer agents in mouse, rat, dog, monkey and man, Cancer Chemoth. Report, 1966, 50(4): 219-244). When administered by continuous intravenous infusion (CIV), the therapeutically effective amount ranges from about 10 mg/m²/day to about 1000 mg/m²/day, from about 20 mg/m²/day to about 750 mg/m²/day, from about 30 mg/m²/day to about 500 mg/m²/day, or about 120 mg/m²/day to about 480 mg/m²/day.

When special requirements may be needed (e.g. for children patients), the therapeutically effective doses described above may be outside the ranges stated herein. Such higher or lower doses are within the scope of the present invention.

To monitor the efficacy of tumor treatment in a human, tumor size and/or tumor morphology is measured before and after initiation of the treatment, and treatment is considered effective if either the tumor size ceases further growth, or if the tumor is reduced in size, e.g., by at least 10% or more (e.g., 20%, 30%, 40%, 50%, 60%, 70%, 80%, 90% or even 100%, that is, the absence of the tumor). Prolongation of survival, time-to-disease progression, partial response and objective response rate are surrogate measures of clinical activity of the investigational agent. Tumor shrinkage is considered to be one treatment-specific response. This system is limited by the requirement that patients have visceral masses that are amenable to accurate measurement. Methods of determining the size of a tumor in vivo vary with the type of tumor, and include, for example, various imaging techniques well known to those in the medical imaging or oncology fields (MRI, CAT, PET, etc.), as well as histological techniques and flow cytometry. For certain types of cancer, evaluation of serum tumor markers are also used to evaluate response (e.g. prostate-specific antigen (PSA) for prostate cancer, and carcino-embryonic antigen (CEA), for colon cancer). Other methods of monitoring cancer growth include cell counts (e.g. in leukemias) in blood or relief in bone pain (e.g. prostate cancer).

The farnesyl dibenzodiazepinone formulation may be administered once daily, or the compound may be administered as two, three, four, or more sub-doses at appropriate intervals throughout the day. In that case, the amount of the farnesyl dibenzodiazepinone contained in each sub-dose must be correspondingly smaller in order to achieve the total daily dosage. The dosage unit can also be compounded for delivery over several days, e.g., using a conventional sustained release formulation which provides sustained release of the farnesyl dibenzodiazepinone compound over a several day period. Sustained release formulations are well known in the art. In this embodiment, the dosage unit contains a corresponding multiple of the daily dose. The effective dose can be administered either as a single administration event (e.g., oral, topical or intranasal administration or bolus parenteral injection) or as a slow injection or continuous infusion, e.g. over 30 minutes to about 24 hours. The formulation may be administered as a treatment, e.g. for up to 30 days. Moreover, treatment of a subject with a therapeutically effective amount of a composition can include a single treatment or a series of treatments (e.g., a four-week treatment repeated 3 times, with a 2 months interval between each treatment). Estimates of effective dosages, toxicities and in vivo half-lives for the farnesyl dibenzodiazepinone compounds encompassed by the invention can be made using conventional methodologies or on the basis of in vivo testing using an appropriate animal model.

Treatment of tumor in a subject, including mammals and humans, may be accomplished by administering the formulation of the invention as a single agent, or in combination with other known anticancer treatments such as radiotherapy and chemotherapy regimen. The farnesyl dibenzodiazepinone may be administered in conjunction with or in addition to known anticancer compounds or chemotherapeutic agents. Chemotherapeutic families include: cytostatic or cytotoxic agents, antibiotic-type agents, alkylating agents, antimetabolite agents, hormonal agents, aromatase agents, immunological agents, interferon-type agents, cyclooxygenase inhibitiors (e.g. COX-2 inhibitors), matrix metalloprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other CDK inhibitors, tubulin binding agents, topoisomerase I inhibitors, topoisomerase II inhibitors, and the like. Examples of chemotherapeutic agents include, but are not limited to, 5-flurouracil, mitomycin C, methotrexate, hydroxyurea, cyclophosphamide, dacarbazine, mitoxantrone, anthracyclins (Epirubicin and Doxurubicin), CPT-11, camptothecin and derivatives thereof, etoposide, navelbine, vinblastine, pregnasome, platinum compounds such as carboplatin and cisplatin, taxanes such as taxol and taxotere; hormone therapies such as tamoxifen and anti-estrogens; antibodies to receptors, such as herceptin and Iressa; aromatase inhibitors, progestational agents and LHRH analogs; biological response modifiers such as IL2 and interferons; multidrug reversing agents such as the cyclosporin analog PSC 833, optionally within liposomal formulations. (For more examples, see: The Merck Index, 12^{th} edition (1996), Therapeutic Category and Biological Activity Index, lists under “Antineoplastic” sections, incorporated herein by reference).

Toxicity and therapeutic efficacy of farnesyl dibenzodiazepinone compounds can be determined by standard pharmaceutical procedures in cell cultures or experimental animals. Therapeutic efficacy is determined in animal models as described above and in the examples herein. Toxicity studies are done to determine the lethal dose for 10% of tested animals (LD10). Animals are treated at the maximum tolerated dose (MTD): the highest dose not producing mortality or greater than 20% body weight loss. The effective dose (ED) is related to the MTD in a given tumor model to determine the therapeutic index of the compound. A therapeutic index (MTD/ED) close to 1.0 has been found to be acceptable for some chemotherapeutic drugs, a preferred therapeutic index for classical chemotherapeutic drugs is 1.25 or higher.

The data obtained from cell culture assays and animal studies can be used in formulating a range of dosage for use in humans. The dosage of compositions of the invention will generally be within a range of circulating concentrations that include the MTD. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound. Such information can be used to more accurately determine useful doses in humans. Levels in plasma may be measured, for example, by high performance liquid chromatography.

Animal models to determine antitumor efficacy of a compound are generally carried out in mice. Either murine tumor cells are inoculated subcutaneously into the hind flank of mice from the same species (syngeneic models) or human tumor cells are inoculated subcutaneously into the hind flank of severe combined immune deficient (SCID) mice or other immune deficient mouse (nude mice) (xenograft models).

Advances in mouse genetics have generated a number of mouse models for the study of various human diseases including cancer. The MMHCC (Mouse models of Human Cancer Consortium) web page, sponsored by the National Cancer Institute, provides disease-site-specific compendium of known cancer models, and has links to the searchable Cancer Models Database, as well as the NCI-MMHCC mouse repository. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of farnesyl dibenzodiazepinone compounds, as well as for determining a therapeutically effective dose.

In addition, formulations of this invention comprising pharmaceutically acceptable salts or prodrugs of farnesyl dibenzodiazepinones may also be employed in compositions to treat or prevent the above-identified disorders.

### EXAMPLES

The formulations exemplified herein were prepared using substantially pure Compound 1, which was isolated from the fermentation broth of either strains of Micromonospora [S01]046 or 046-ECO11 respectively having IDAC 231203-01 and 070303-01 accession numbers (International Depository Authority of Canada (IDAC), Bureau of Microbiology, Health Canada, 1015 Arlington Street, Winnipeg, Manitoba, Canada, R3E 3R2). Compound 1 was produced and isolated according to the procedures described in U.S. patent application Ser. No. 10/762,107 filed Jan. 21, 2004, also published as WO 2004/065591 in August 2004, incorporated herein by reference in their entirety. Any compound of Formula I may replace Compound 1 in the formulations of this invention. The compounds of Formula I, including Compounds 1 to 11, 14, 17, 18, 46, 63, 64, 67, 77, 78, 80, 82 to 85, 87, 89, 92, 95 to 98, 100 to 103, 105, 107 and 108, were prepared according to the procedures disclosed in U.S. Publication Number US 2006/0079512.

Unless otherwise indicated, all reagents, solvents, or excipients were supplied by Sigma-Aldrich, or Fisher Scientific. Kollidon™ 12 PF (PVP), Lutrol™ E400 (PEG 400) and Cremophor™ EL were supplied by BASF. Lipids (EPC, DMPC and PEG₂₀₀₀DSPE), and cholesterol were supplied by Northern Lipids or Avanti® Polar Lipids.

Unless otherwise indicated, all numbers expressing quantities of ingredients, properties such as stability and solubility, pharmacokinetic results, efficacy results, GI₅₀ and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set in the examples, Tables and Figures are reported as precisely as possible. Any numerical values may inherently contain certain errors resulting from variations in experiments, testing measurements, statistical analyses and such.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### Example 1

### Bulk Surfactant Formulations A, B and C

Formulations A, B and C were prepared by the procedure described below. Table 1 summarizes the different ingredients and respective proportions used for their preparation. The same formulations may be produced in larger quantities using large-scale methods and equipment known to the art, and keeping the same average proportions as the formulations of this invention.

TABLE 1
Bulk Formulations A, B and C ingredients for 5 mg of Compound 1

| | A | B | C |
|---|---|---|---|
| Compound 1 | 5 mg | 5 mg | 5 mg |
| Polysorbate 80 | 88 mg | 88 mg | — |
| PEG 400 | — | 25 mg | — |
| PVP | 25 mg | — | — |
| Cremophor ™ EL | — | — | 125 mg |
| Ethanol | 76 μL | 59 μL | 75 μL |

1. Formulation A

The appropriate number of serum bottles (USP type 1; borosilicate; clear; size of 2 mL, 5 mL, 10 mL or 30 mL) and Teflon™-coated butyl stoppers were autoclaved at 121° C. in an autoclave bag for 15 minutes.

A stock solution of Compound 1 in ethanol was prepared (250 mg/mL), in a volumetric flask. A stock solution of PVP (Kollidon™ 12 PF) was prepared in ethanol (450 mg/mL), in a volumetric flask. An amount of polysorbate 80 (1401 mg) was weighed in a 20 mL scintillation vial. The PVP solution (893 μL, containing 402 mg of PVP) was added to the vial and the mixture vortexed for 30 seconds. Compound 1 solution (320 μL, containing 80 mg) was added and the mixture vortexed for 30 seconds. The mixture was sterilized by filtration (0.2 micron NL16 S&S sterile filter, supplied by Schleicher & Schuell) in a sterile environment to give a bulk formulation containing 80 mg of Compound 1 ready for reconstitution. Formulation A may also be used as it is for oral administration.

In a sterile environment, a volume containing 5 mg of Compound 1 (see Table 1) was added to each sterile serum bottle and the bottles closed with Teflon™-coated stoppers, and sealed with aluminium seals.

2. Formulation B

The appropriate number of serum bottles (USP type 1; borosilicate; clear; size of 2 mL, 5 mL, 10 mL or 30 mL) and Teflon™-coated butyl stoppers were autoclaved at 121° C. in an autoclave bag for 15 minutes.

A stock solution of Compound 1 in ethanol was prepared (250 mg/mL), in a volumetric flask. A stock solution of PEG 400 (Lutrol™ E400) was prepared in ethanol (650 mg/mL), in a volumetric flask. An amount of polysorbate 80 (1401 mg) was weighed in a 20 mL scintillation vial. The PEG 400 solution (618 μL, containing 402 mg of Lutrol™) was added to the vial and the mixture vortexed for 30 seconds. Compound 1 solution (320 μL, containing 80 mg) was added and the mixture vortexed for 30 seconds. The mixture was sterilized by filtration (0.2 micron NL16 S&S sterile filter) in a sterile environment to give a bulk formulation B containing 80 mg of Compound 1 ready for reconstitution. Formulation B may also be used as it is for oral administration.

In a sterile environment, a volume containing 5 mg of Compound 1 (see Table 1) was added to each sterile serum bottle and the bottles closed with Teflon™-coated stoppers, and sealed with aluminium seals.

3. Formulation C

An amount of 120 mg was dissolved in 1.8 mL of ethanol, and 3 g of Cremophor™ EL was added. The solution was vortexed for 30 seconds. The mixture was used as is, for oral administration.

### Example 2

### Bulk Surfactant Formulations B1 to B10

A. Formulations B1 to B9

Preparation:

Formulations B1 to B9 were prepared by following the method of Formulation B (Example 1-A-2). For Formulations B6 to B9, water and sodium ascorbate with or without ascorbic acid were further added. B1 to B3 were also used as control formulations, where only one excipient at a time was used, to verify the effect of each on drug stability. Formulation B4 corresponded to Formulation B above, with a lower content in ethanol (B4 contains about 25 μL of ethanol per 5 mg of drug). Formulation B5 contained sodium ascorbate as antioxidant agents. Formulations B6 to B8 contained increasing water content but the same ascorbic acid/sodium ascorbate content, acting as buffer and antioxidant. Table 2 summarizes the different ingredients and respective proportions used for their preparation.

TABLE 2
Bulk Formulations B1 to B9 (% wt)

| | B1 | B2 | B3 | B4 | B5 | B6 | B7 | B8 | B9 |
|---|---|---|---|---|---|---|---|---|---|
| Compound 1 | 20.20 | 4.45 | 10.05 | 3.64 | 3.47 | 3.47 | 3.36 | 3.15 | 3.18 |
| Polysorbate 80 | — | 77.95 | — | 63.75 | 60.81 | 60.81 | 58.77 | 55.16 | 55.73 |
| PEG 400 | — | — | 50.25 | 18.21 | 17.37 | 17.37 | 16.79 | 15.76 | 15.92 |
| Ethanol | 79.80 | 17.59 | 39.70 | 14.39 | 13.73 | 13.73 | 13.26 | 12.45 | 12.58 |
| Ascorbic acid | — | — | — | — | — | 0.57 | 0.55 | 0.52 | — |
| Sodium ascorbate | — | — | — | — | 1.14 | 0.57 | 0.55 | 0.52 | — |
| Water | — | — | — | — | 3.47 | 3.47 | 6.72 | 12.45 | 12.58^{a} |

^{a}2N hydrochloric acid solution

Stability:

Aim of the study was to verify the relative stability and effect of water, ascorbic acid and sodium ascorbate to prevent drug degradation. Bulk Formulations B1 to B9 were kept at temperatures of about 5° C., about 25° C. (±2° C., relative humidity (RH) of 60%), and about 40° C. (±2° C., RH of 70%), in the upright position protected from light. Drug content was tested by HPLC after 1, 2, 3 weeks, and 2 months. Formulations B5 and B8 were also tested after 4 months. Results are shown in Table 3 below.

TABLE 3
Stability testing result for Formulations B1 to B9

| | | Time | | | | |
|---|---|---|---|---|---|---|
| Temperature (° C.) | Formulation | 1 week | 2 weeks | 3 weeks | 2 months | 4 months |
| 5 ± 3° C. | B1 | 95.18 | 99.21 | 95.4 | 99.52 | — |
| | B2 | 97.16 | 95.49 | 97.4 | 93.92^{a} | — |
| | B3 | 102.04 | 99.92 | 101.3 | 100.73 | — |
| | B4 | 97.84 | 95.22 | 96.0 | 91.56^{a} | — |
| | B5 | 102.31 | 100.63 | 103.0 | 101.44 | 101.74 |
| | B6 | 100.70 | 99.82 | 100.3 | 100.12 | — |
| | B7 | 101.56 | 100.37 | 102.4 | 101.84 | — |
| | B8 | 102.07 | 101.65 | 103.2 | 101.71 | 103.69 |
| | B9 | 103.26 | 100.28 | 103.2 | 100.68 | — |
| 25° C. ± 2° C. | B1 | 91.42^{a} | 96.80 | 97.7 | 85.51^{b} | — |
| RH 60% ± 5% | B2 | 92.26^{a} | 87.42^{b} | 86.7^{b} | 69.26^{b} | — |
| | B3 | 100.46 | 101.97 | 102.6 | 96.74 | — |
| | B4 | 88.67^{b} | 83.02^{b} | 80.3^{b} | 71.08^{b} | — |
| | B5 | 106.29^{a} | 101.17 | 102.9 | 101.44 | 100.54 |
| | B6 | 99.91 | 98.27 | 100.4 | 99.53 | — |
| | B7 | 100.86 | 100.12 | 103.8 | 101.84 | — |
| | B8 | 102.14 | 102.11 | 102.6 | 102.04 | 102.70 |
| | B9 | 97.36 | 90.22^{a} | 90.1^{a} | 66.80^{b} | — |
| 40° C. ± 2° C. | B1 | 91.28^{a} | 92.15^{a} | 86.6^{b} | 61.94^{b} | — |
| RH 70% ± 5% | B2 | 78.78^{b} | 70.14^{b} | 66.5^{b} | 51.30^{b} | — |
| | B3 | 100.37 | 100.12 | 98.3 | 87.61^{b} | — |
| | B4 | 76.32^{b} | 68.98^{b} | 66.2^{b} | 53.45^{b} | — |
| | B5 | 100.87 | 99.10 | 100.7 | 96.34 | 89.44^{b} |
| | B6 | 99.06 | 97.30 | 99.6 | 93.95^{a} | — |
| | B7 | 101.04 | 99.85 | 102.6 | 98.16 | — |
| | B8 | 101.02 | 99.84 | 102.0 | 97.43 | 96.12 |
| | B9 | 74.49^{b} | 41.19^{b} | 25.8^{b} | 0.00^{b} | — |

^{a}Outside 95-105%
^{b}Outside 90-110%

HPLC analysis showed that drug degradation of the initial Formulation (B4) followed the same trend then the drug in presence of only polysorbate 80, which indicates that polysorbate 80 is the main excipient which may cause drug degradation. This trend clearly amplified as a function of increasing storage temperatures.

As shown in Table 3, the Formulation B1 featured a slight decrease of the drug content when stored at 25 and 40° C., whereas no degradation was observed when kept refrigerated. Ambient temperature induced Compound 1 degradation in a highly polar and protic solvent such as ethanol. A previous study showed that Compound 1 slightly oxidizes when dissolved in methanol.

Formulation B9, containing 12.58% of a 2N hydrochoric acid solution, displayed significant drug degradation at ambient temperature with drug loss of up to 40% over 2 months, whereas complete drug degradation was observed at 40° C. after the same period. Nevertheless, the drug content of Formulation B9 remained stable when stored refrigerated.

The drug content of all formulation B5 to B8 remained within the range of 95-105% of the initial content over 2 months. However, a stability difference was observed between B5 and B8 when stored at 40° C. for 4 months. In this particular case, the higher water content might contribute to the prevention of drug degradation.

Formulations B2 and B4, comprising polysorbate 80 and ethanol without antioxidant was less stable when stored at room temperature, or at 5° C. for 2 months or more. When these formulations are used, they would preferably be prepared prior to reconstitution or be kept at low temperature for a short period of time.

The presence of sodium L-ascorbate prevented drug degradation at temperatures up to 40° C. for up to 4 months in Formulation B5. According to ICH guidelines (International Conference on Harmonisation of Technical Requirements for Registration of Pharmaceuticals for Human Use, Guideline Q1A(R2), February 2003, incorporated by reference in its entirety), it can be expected from these results that the drug will be stable for at least 8 months at ambient temperature, and for 12 months when refrigerated. The presence of water seemed to also delay the drug degradation. The addition of water into the formulation composition allowed for the only use of sodium L-ascorbate (salt) as antioxidant agent.

Based on results, water and sodium L-ascorbate are preferred for preventing drug degradation and extending shelf life time of surfactant formulations as herein described. The quantity of water should be equalled to the one of ethanol. The sodium L-ascorbate should be equalled to the maximum free fatty acids that might be present in the Compound 1 drug product because of the polysorbate 80.

B. Bulk Formulation B10

Preparation:

Formulation B10 was produced by mixing, by weight: Compound 1 (3.15%), polysorbate 80 (Crillet 4 HP™, 55.15%), PEG 400 (15.76%), ethanol absolute (12.45%), water (water-for-injection (WFI), 12.45%), and (+)-sodium L-ascorbate (1.04%). Polysorbate 80 was obtained from J. T. Baker, absolute ethanol from Commercial Alcohols Inc., PEG 400 and (+)-sodium L-ascorbate from Spectrum, and WFI from VWR.

The bulk Formulation B10 was prepared according to the following: Compound 1 was dissolved in ethanol and filter sterilized (0.22-micron PES (polyethersulfone) membrane filter) producing solution A. (+)-Sodium L-ascorbate was dissolved in water-for-injection and filter sterilized (0.22-micron PES) producing solution B. Polyethylene glycol was added to polysorbate 80 (both sterilized by dry heat at 160-165° C. for five hours) producing solution C. Aseptically, solution A and solution B were successively added to solution C. The final bulk solution B10 was filter-sterilized through a sterile Millipak-60™ cartridge.

Stability:

Formulation B10 was assayed for stability at 5±3 and 25±2° C. (60±2% relative humidity). The content in active ingredient at time zero was 95.0%. After 2 months at 5±3° C., the content in active ingredient was 97.3%. After 2 months at 25±2° C., the content in active ingredient was 96.7%. The formulation is expected to be the most stable as it contain sodium ascorbate (as in B5) and water (as in B8), the two most stable formulations of Tables 2 and 3.

### Example 3

### Ready-to-Use Surfactant Formulations D1 to D11

The following D1 to D11 formulations were also prepared. Final dilution was done in isotonic medium, at 10 and 1 mg/mL of Compound 1. Isotonic media used were both 0.9% saline and 5% dextrose. Table 4 summarizes the compositions used in 10 mg/mL formulations. The procedure used to produce all of them is described below.

TABLE 4
Composition (% w/v) of Formulations D1 to D11
(10 mg/mL of Compound 1)

| | Polysorbate 80 | PEG 400 | PVP | Ethanol (% v/v)^{a} |
|---|---|---|---|---|
| D1 | 25 | — | — | <8 |
| D2 | 20 | — | 2.5 | <8 |
| D3 | 15 | — | 2.5 | <8 |
| D4 | 20 | — | 5 | <8 |
| D5 | 15 | — | 5 | <8 |
| D6 | 20 | — | 7.5 | <8 |
| D7 | 15 | — | 7.5 | <8 |
| D8 | 10 | — | 7.5 | <8 |
| D9 | 20 | 7.5 | — | <8 |
| D10 | 15 | 7.5 | — | <8 |
| D11 | 15 | 5 | — | 5 |

^{a}ethanol content from the final formulations is <8% (v/v), which is less than the recommended maximum 10% (v/v).

Stock Solutions:
Compound 1 (obtained as described above) at 250 mg/mL in ethanol
Polysorbate 80 (Tween™ 80, Sigma-Aldrich) 750 mg/mL in ethanol
PVP (Kollidon™ 12 PF—polyvinylpyrrolidone) 700 mg/mL in ethanol
PEG 400 (Lutrol™ E400) used as it is.

A volume of 20 μL (containing 5 mg) of the Compound 1 solution was added to a culture tube (13×100 mm). A solution of polysorbate 80 was added, according to the desired amount (see Table 4), and PVP or PEG 400, where appropriate (see Table 4). The solution was vortexed for 10 seconds between each addition. Isotonic medium (0.9% saline or 5% dextrose) was added to reach a concentration of 10 mg/mL of Compound 1 and the solution shaken for 3 minutes by hand. A volume of 100 μL of the 10 mg/mL solution was transferred in a second tube and an extra volume of 900 μL of isotonic media was added to reach a 1 mg/mL and the solution shaken for 3 minutes by hand.

Formulations D1-D10 all resulted in clear solutions and the drug stayed in solution for at least 6 hours, both at 10 mg/mL and 1 mg/mL. Formulation D11 resulted in a clear solution and the drug stayed in solution for at least 6 hours, at all concentrations between 10 mg/mL and 1 mg/mL.

Ready-to-use Formulation D11 (6 or 10 mg/mL concentration) was also prepared by reconstitution, with D5W (5% dextrose), of a bulk formulation (Formulation B4) containing, 20% ethanol (v/v), 20% PEG 400 (w/v) and 60% polysorbate 80 (w/v), and having a concentration of 24 or 40 mg/mL of Compound 1.

Formulation D11 was also prepared by replacing Compound 1 by Compound 2 (Formulation D11(2)) or Compound 46 (Formulation D11 (46)) as active ingredient. Both Formulations D11(2) and D11(46) resulted in clear solutions and their respective active ingredient stayed in solution for at least 6 hours, at all concentrations between 10 mg/mL and 1 mg/mL. These formulations were used in in vivo studies.

### Example 4

### Liposomal Formulations E1 to E25

Liposomal formulations of Compound 1 were produced using various phospholipids with or without cholesterol. Abbreviations have the following meaning:
API: Active Pharmaceutical Ingredient (here Compound 1, MW: 462.6)
EPC: Egg phosphatidylcholine (MW: 386.6)
DMPC: Dimyristoylphosphatidylcholine (MW: 677.9)
PEG₂₀₀₀DSPE: Distearoylphosphatidylethanolamine-PEG (MW: 2810.3)
Chol: Cholesterol (MW: 386.6)

Phospholipids and cholesterol were supplied by Northern Lipids and Avanti® Polar Lipids. Active ingredient Compound 1 was prepared according to patent applications as mentioned in Example 1. Table 5 summarises the concentrations of ingredient used in each formulation.

TABLE 5
Formulations E1 to E25 constituents and final drug concentration (mg/mL)

| | | | Concentration | |
|---|---|---|---|---|
| | Constituents | Relative Molar Ratio | (mM)^{a} | Drug (mg/mL)^{b} |
| E1 | EPC:API | 92.5:7.5 | 30 | 1.04 |
| E2 | EPC:API | 92.5:7.5 | 60 | 2.08 |
| E3 | EPC:API | 90:10 | 30 | 1.39 |
| E4 | EPC:API | 90:10 | 60 | 2.78 |
| E5 | EPC:API | 80:20 | 60 | 5.55 |
| E6 | EPC:Chol:API | 76:19:5 | 60 | 1.39 |
| E7 | EPC:DSPE-PEG:API | 89.5:3:7.5 | 30 | 1.04 |
| E8 | EPC:DSPE-PEG:API | 89.5:3:7.5 | 60 | 2.08 |
| E9 | EPC:DSPE-PEG:API | 86.9:5.8:4.7 | 60 | 2.08 |
| E10 | EPC:DSPE-PEG:API | 86.5:6:7.5 | 30 | 1.04 |
| E11 | EPC:DSPE-PEG:API | 86.5:6:7.5 | 60 | 2.08 |
| E12 | EPC:DSPE-PEG:API | 89:6:5 | 60 | 1.39 |
| E13 | EPC:DSPE-PEG:API | 79:6:15 | 60 | 4.16 |
| E14 | EPC:DSPE-PEG:API | 74:6:20 | 60 | 5.55 |
| E15 | EPC:Chol:DSPE-PEG:API | 67.2:16.8:6:10 | 60 | 2.78 |
| E16 | EPC:Chol:DSPE-PEG:API | 63.2:15.8:6:15 | 60 | 4.16 |
| E17 | DMPC:API | 92.5:7.5 | 30 | 1.04 |
| E18 | DMPC:Chol:API | 76:19:5 | 60 | 1.39 |
| E19 | DMPC:Chol:API | 83.3:9.33:7.5 | 60 | 2.08 |
| E20 | DMPC:Chol:API | 74.0:18.5:7.5 | 60 | 2.08 |
| E21 | DMPC:DSPE-PEG:API | 86.5:6:7.5 | 30 | 1.04 |
| E22 | DMPC:DSPE-PEG:API | 86.5:6:7.5 | 60 | 2.08 |
| E23 | DMPC:Chol:DSPE-PEG:API | 67.2:16.8:11.3:4.7 | 60 | 1.39 |
| E24 | DMPC:Chol:DSPE-PEG:API | 67.2:16.8:6:10 | 60 | 2.78 |
| E25 | DMPC:Chol:DSPE-PEG:API | 63.2:15.8:6:15 | 60 | 4.16 |

^{a}Total molar concentration (mM) of all-combined components
^{b}Drug Compound 1 concentration assuming 100% incorporation of the drug added.

Liposomal formulations E1 to E25 were prepared according to the following procedure:

A stock solution of Compound 1 (50 mg/mL) was prepared in a mixture of methanol/chloroform (1:1). Stock solutions of each lipid (EPC, DMPC and DSPE-PEG (i.e. PEG₂₀₀₀DSPE)) were prepared as 3 separate 40 mg/mL solutions, using the same solvent system. Finally a stock solution of cholesterol was also prepared at 40 mg/mL using the same solvent system. Required volume* of lipids (EPC, DMPC and PEG₂₀₀₀DSPE), cholesterol and active ingredient (Compound 1) stock solutions were combined in culture tubes (12×75 mm) or in round-bottom flasks if the volume was higher than 1 mL.

*For example of required volume: Formulation E5, having a desired total hydrated molar concentration of 60 mM, and a molar ratio of components of 80:20 (EPC/API), required a total of 0.06 mmole of material (for a 1 mL scale), i.e. 0.048 mmole EPC and 0.012 mmole API, which gave a required 0.91 mL and 0.11 mL of their respective stock solutions.

The resulting solution was gently mixed. The solvent was removed by rotary evaporation and residual traces of solvent evaporated in vacuo for at least 4 hours, preferably overnight.

Hydration was done by the addition of a 5% (w/v) dextrose solution to the tube (1 mL). Hydration was done at a temperature above the Tₘ of the lipid, for example EPC-only (Tₘ=−2.5° C.) formulations were hydrated at room temperature and DMPC formulations (Tₘ=23° C.) were hydrated at 30° C., in a water bath set at the desired temperature. The lipid/drug mixture was suspended by vigorous mixing using a vortex, until no residual film was observed. A sonicator bath was also used when necessary (alternating sonication and vortex).

The suspension was hydrated at 4° C. overnight, allowing non-incorporated drug, if any, to precipitate or crystallize. Liposomal suspension was extruded using an Avanti™ Mini-Extruder from Avanti Polar Lipids (with at least 500 μL of liposomes and 1 mL syringes). Liposome suspension was passed 21 times through a 100 nm polycarbonate filter, and 21 times through a 50 nm filter. For both 100 nm and 50 nm extrusions, the suspension was collected at the opposite side from which extrusion started (to allow removal of precipitated drug) and the extruder rinsed.

A 10 mL Lipex™ extruder (Northern Lipids) was used when more than 1 mL of liposomes was prepared. The extrusion was done using nitrogen gas, 10 times through a 100 nm filter, 10 times through a 50 nm filter, or until desired liposome size was achieved.

Liposomes were sterilized by filtration through a 0.2 μm sterile filter in a sterile hood and kept at 4° C. Formulations were characterized to determine liposome size by measuring the Brownian motion of particles by Dynamic Light Scattering (DLS, measured in 5% dextrose, using a Malvern NanoSizer NS™, in automatic mode). Brownian motion is the random movement of particles in a fluid due to the bombardment by the molecules that surround them. For formulations E1-4, 6-12, 15, 17 and 18-24, the average diameter of the liposomes was comprised between 102-190 nm. The average liposomes diameter for formulations E5, 13-14, 16 and 25 was included between 120-165 nm. Liposome sizes did not change upon storage for at least 3 weeks.

### Example 5

### Formulation F

Formulation F, as described in U.S. application Ser. No. 10/951,436 filed Sep. 27, 2004, was produced by dissolving Compound 1 in a 30:30:40 solution of PEG/PG/water. PG is propylene glycol and was supplied by Sigma-Aldrich. The concentration of Compound 1 was adjusted by dissolving the appropriate amount in the solution and the formulation obtained was used as is. For example, to obtain a 20 mg/mL solution, 20 mg of Compound 1 were dissolved per mL of the above solution.

### Example 6

### Pharmacokinetic Properties of Formulations B and F in CD1 Mice

A. Pharmacokinetics:

Table 6 summarizes key results obtained from administration of Formulation F (30 and 50 mg/kg) and reconstituted Formulation B (at 30 mg/kg), including Cₘₐₓ, Tₘₐₓ and AUC. Cₘₐₓ values represent the maximum observed plasma concentration, Tₘₐₓ values represent the time where the maximum concentration was observed, and AUC represents the area under the plasma concentration versus time curve.

TABLE 6
Compound 1 Pharmacokinetics for Formulations B and F in CD1 mice

| | | Tₘₐₓ | AUC (ng/mL * H) |
|---|---|---|---|
| Formulation/Mode/Dose | Cₘₐₓ (ng/mL) | (H) | 0.033 → 4.0 H^{a} |
| B, IV (30 mg/kg) | 131452.0 | 0.033 | 18457 |
| F, IV (30 mg/kg) | 46916.7 | 0.033 | 5550 |
| F, IV (50 mg/kg) | 50750.0 | 0.033 | 8956 |
| F, 30 minutes IV short | 27500.0 | 0.33 | 17454 |
| infusion (50 mg/kg) | | | |

^{a}AUC (ng/mL*H) between 0.033 → 2.5 H for IV infusion (T₀ is the start of infusion).

Bulk Formulation B was reconstituted using 5% dextrose, to reach a 3 mg/mL concentration, used for pharmacokinetic (PK) studies. Formulation F was produced by dissolving 20 mg per mL of the 30:30:40 solution of PEG/PG/water and used as is (20 mg/mL) for the PK testing.

CD1 female mice (6 weeks of age) received a single intravenous (30 mg/kg; 10 mL/kg) dose of Compound 1 in Formulation B described above and either a single intravenous dose of Formulation F (30 and 50 mg/kg; 1.5 and 2.5 mL/kg) or a 30-minute infusion of Formulation F (50 mg/kg; 2.5 mL/kg). Four (4) mice per group were sacrificed at 3 min, 5 min, 15 min, 30 min, 1 h, 2 h, 4 h and 8 h. Blood was collected into EDTA-containing tubes by cardiac puncture and brains were rapidly collected and immediately frozen on dry ice. Samples were analysed by LC/MS/MS. Standard curve ranged from 25 to 2000 ng/mL with limit of quantification (LOQ)≦15 ng/mL. Plasma and brain concentration values of Compound 1 falling below the limit of quantification (LOQ) were set to zero. Mean concentration values and standard deviation (SD) were calculated at each time points of the pharmacokinetic study (n=4 animals/time point).

The PK study showed a significant increase of the maximum plasma concentration (Cₘₐₓ) of about 2.8-fold, for reconstituted Formulation B compared to Formulation F. Also, the AUC tripled between 3 minutes and 8 hours, for Formulation B versus Formulation F, at the same dosage (30 mg/kg).

B. Tissue Distribution:

The drug accumulation in mouse brain using Formulation B (reconstituted as in A), was compared to formulation F at the same dose (30 mg/kg). No haemorrhage or inflammation was observed in mouse brain tissues with formulation B. A capillary inflammation was observed with formulation F at doses of 30 and 50 mg/kg.

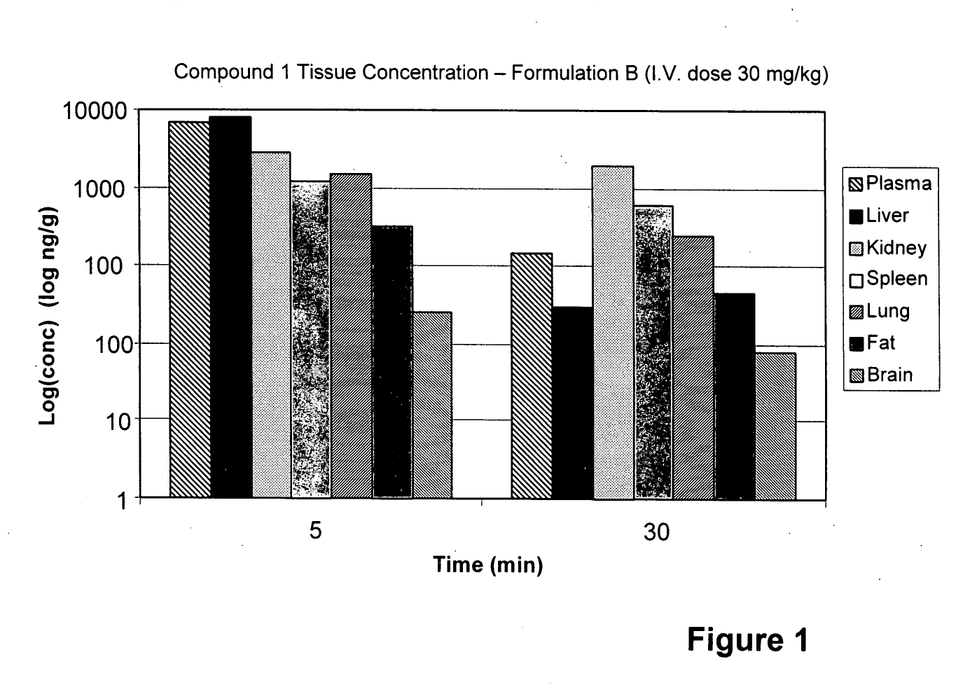

Various tissues were dosed at time points of 5 and 30 minutes, as illustrated in FIG. 1. Although the drug level in plasma dropped of two orders of magnitude after 30 minutes, the drug concentration in brain tissue remained relatively constant. The ratio of drug concentration in brain to that in fat was constant, indicating that the blood-brain barrier did not seem to restrain drug crossing into brain tissue.

### Example 7

### Oral Bioavailability of Formulations C and F in Mice

No dilution is required for Formulation C, and was produced as described in Example 1. Compound 1 concentration was 26 mg/mL, and the administered dose was 120 mg/kg.

Formulation F was produced according to Example 4, as a 20 mg/mL solution in 30:30:40 PEG/PG/water. The administered dose was 120 mg/kg.

Both Formulations were used for PO (per os, oral) administration by gavage (mice). The administration volume was adjusted as a function of individual mouse weight. The AUC (PO) results were determined as described in Example 6.

Oral bioavailability (F), was determined using the following formula:

$$F = \frac{AUC ( PO )}{AUC ( IV )} \times \frac{dose ( IV )}{dose ( PO )}$$

wherein AUC (IV) and dose (IV) values correspond to the results obtained for IV administration of reconstituted Formulation B, as described in Example 6. At a dosage of 120 mg/kg, oral bioavailability of Formulation C was 3.4% compared to oral bioavailability of Formulation F, which was 2.6%.

### Example 8

### Toxicity of Formulations B and F in CD1 Mice

With CD1 mice, the maximum tolerated dose (MTD) for a single-dose IV injection of Formulation F was 100 mg/kg (10 mg/mL concentration, in PEG/PG/Water 30:30:40). Using this dosage, oedema and necrosis at the injection site was observed.

With CD1 mice, the maximum tolerated dose (MTD) for a single-dose IV injection of reconstituted Formulation B was 150 mg/kg (20 mg/mL concentration, reconstituted in 5% dextrose). A multiple dose regimen of reconstituted Formulation B was well tolerated for up to 150 mg/kg (15 mg/mL concentration, reconstituted in 5% dextrose), when injected once a day over 2 weeks (Q1D×5×2 weeks), without causing any apparent mouse weight loss. The MTTD (maximum total tolerated dose) for reconstituted Formulation B was around 150 mg/kg.

### Example 9

### Pharmacokinetic Studies using Formulation D11

Formulation D11, at a final concentration of 6 mg/ml of Compound 1 was used for IV, IP and SC bolus administration. For oral administration, Formulation C was used at a final concentration of 6 mg/ml in Cremophor EL™/Ethanol (50%:50%). Prior to dosing, animals (female Crl: CD1 mice; 6 weeks of age, 22-24 g) were weighed, randomly selected and assigned to the different treatment groups. Compound 1 was administered by the intravenous (IV), subcutaneous (SC), intraperitoneal (IP), or oral (PO) route to the assigned animals. The dosing volume of Compound 1 was 5 mL per kg body weight. Animals were anesthetized prior to bleeding with 5% isoflurane. Blood was collected into microtainer tubes containing the anticoagulant K₂EDTA by cardiac puncture from each of 4 animals per bleeding timepoint (2 min, 5 min, 15 min, 30 min, 1 h, 2 h, 4 h and 8 h). Following collection, the samples were centrifuged and the plasma obtained from each sample was recovered and stored frozen (at approximately −80° C.) pending analysis. At the 5 min and 30 min time points, the following organs were harvested from each animal: brain, lungs, skeletal muscle, fat tissue, kidneys, spleen, thymus and liver. Tissues were frozen (at approximately −80° C.) pending analysis. Samples were analysed by LC/MS/MS. Standard curve ranged from 25 to 2000 ng/mL with limit of quantitation (LOQ)≦25 ng/mL and limit of detection (LOD) of 10 ng/mL.

Plasma values of Compound 1 falling below the limit of quantitation (LOQ) were set to zero. Mean concentration values and standard deviation (SD) were calculated at each timepoints of the pharmacokinetic study (n=4 animals/timepoint). The following pharmacokinetic parameters were calculated: area under the plasma concentration versus time curve from time zero to the last measurable concentration time point (AUC₀₋ₜ), area under the plasma concentration versus time curve extrapolated to infinity (AUC_{inf}), maximum observed plasma concentration (Cₘₐₓ), time of maximum plasma concentration (tₘₐₓ), apparent first-order terminal elimination rate constant (kₑₗ), apparent first-order terminal elimination half-life will be calculated as 0.693/kel (t_{1/2}). The systemic clearance (CL) of Compound 1 after intravenous administration was calculated using Dose/AUC_{inf}. Pharmacokinetic parameters were calculated using Kinetica™ 4.1.1 (Innaphase Corporation, Philadelphia, Pa.).

Results

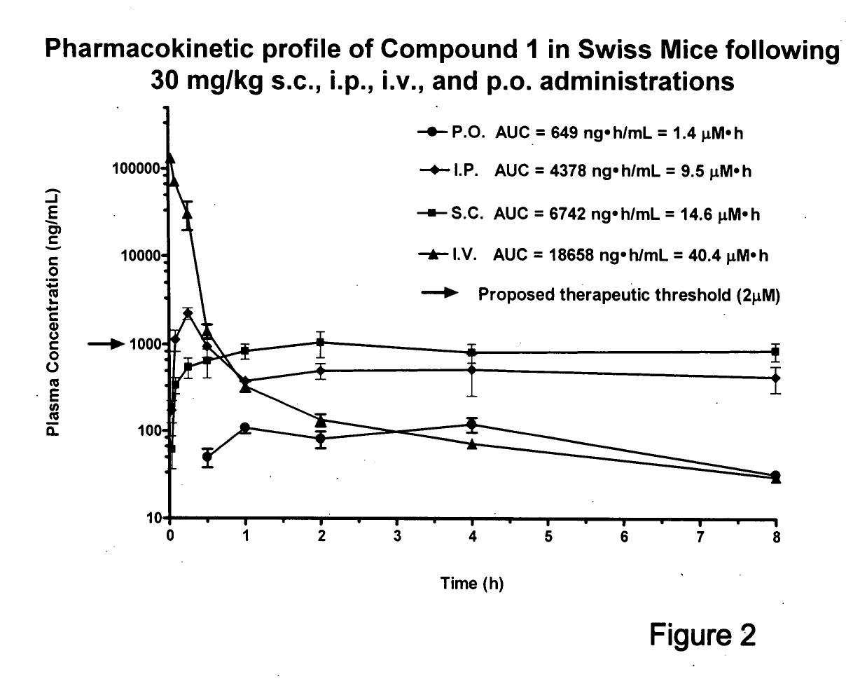

Mean plasma concentrations of Compound 1 following intravenous (IV), intraperitoneal (IP), subcutaneous (SC), and oral (PO) administrations at 30 mg/kg are presented in FIG. 2.

Mean (±SD) plasma concentrations of Compound 1 following IV administration of a 30 mg/kg dose declined rapidly in a biexponential manner resulting in very short half lives (t_{1/2} α and β of 4.6 min and 2.56 h, respectively). On the other hand, the pharmacokinetics of Compound 1 following intraperitoneal and subcutaneous administrations showed a PK profile suggestive of slow release. With both these routes of administration, the compound plasma concentration is sustained and maintained at therapeutically relevant levels for over 8 hours. Oral administration results in moderate but sustained drug levels. These data indicate that Compound 1 is orally bioavailable (˜5-8% when compared to IV bolus administration).

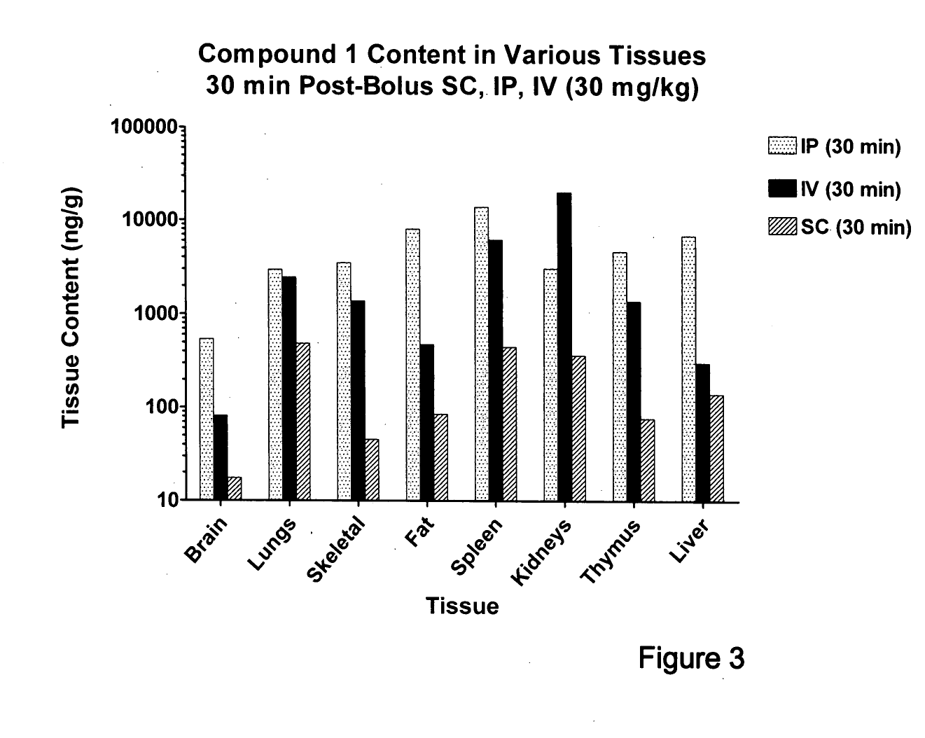

Mean tissue concentrations of Compound 1 30 min after intravenous (IV), intraperitoneal (IP) or subcutaneous (SC) administrations at 30 mg/kg are presented in FIG. 3. The 30 min time point was chosen since plasma concentrations were similar with all three routes of administration. Compound 1 is well distributed following IV and IP dosing. Surprisingly, although IP and SC administrations resulted in a similar PK profile, tissue levels were significantly lower following SC dosing. This could be explained by the absence of peak levels following SC administration compared with IV and IP administrations.

### Example 10

### In Vivo Antitumor Efficacy Studies using Formulation DII

Animal studies were done according to ethical guidelines of animal experimentation (Charte du comité d'éthique du CNRS, 2003) and the English “Guidelines for the welfare of animals in experimental neoplasia (Second Edition)” from the United Kingdom Coordinating Committee on Cancer Research (UKCCCR) (Workman et al. (1998), Br. J. Cancer, vol 77, no 1, 1-10).

A. Rat C6 Glioblastoma Mice Model

The rat C6 glioblastoma antitumor efficacy study was performed at INSERM U318 (Grenoble, France). The rat C6 glioblastoma subcutaneous tumor model is based on the use of a rat C6 cell line obtained from a rat glial tumor induced by N-nitrosomethylurea (Benda et al. (1968), Science, vol 161, 370-371). On each dosing day, Compound 1 stock solutions in bulk Formulation B11 (24 and 40 mg/mL in 20% ethanol, 20% PEG400 and 60% polysorbate 80) were diluted with sterile 5% dextrose in water (D5W) to prepare dosing solutions of 6 mg/mL and 10 mg/mL of Compound 1 in Formulation D11 (5% ethanol, 5% PEG-400, 15% polysorbate 80, and 75% D5W).

For the rat glioma antitumor efficacy study, female athymic (nu/nu) nude mice (6-7 weeks of age) were inoculated SC with 5×10⁶ C6 cells (day 0). Tumor bearing animals were randomized (10 per group) when tumors were palpable (day 6). Group 1 (control group) received drug-free Formulation D11 IP (5 mL/kg), once daily on days 6-18 (q1d×13). Group 2 received Compound 1 (6 mg/mL) IP at 20 mg/kg, once daily on days 6 to 13 and then at 10 mg/kg once daily on days 14 to 18. Group 3 received Compound 1 (6 mg/mL) SC at 30 mg/kg, once daily on days 6 to 13 and then at 15 mg/kg once daily on days 14 to 18. Group 4 received Compound 1 (10 mg/mL) IV at 100 mg/kg q1d×5 for 2 weeks. Each animal was euthanized when its tumor reached the predetermined endpoint size (˜2,500 mm³) or at the end of the study (D18). Treatment period was over 13 days, from day 6 to day 18, post tumor cell inoculation. Tumor growth inhibition (TGI) was calculated on day 16 post tumor cell inoculation, at which time some animals from the vehicle control group had to be sacrificed due to tumor burden.

Determination of Antitumor Activity:

Tumor growth was followed every other day by measuring tumor length (L) and width (W) using a calliper. Measurements were converted to tumor volumes (TV; mm³) using the standard formula, TV=(L×W²)/2. Tumor volume at day n was expressed as relative tumor volume (RTV) according to the following formula RTV=TVₙ/TV₀, where TVₙ is the tumor volume at day n and TV₀ is the tumor volume at day 0. The percentage of tumor growth inhibition (% TGI) was determined by 1−(mean RTV of treated group/mean RTV of control group)×100. According to the NCI standards, a % TGI of ≧58% (T/C≦42%) is indicative of antitumor activity. Statistical analysis was calculated by the two-tailed unpaired t test using the Prism software. Animals were weighed at least twice weekly during and after treatment until completion of the study. The mice were examined frequently for overt signs of any adverse drug-related side effects. Animals were euthanized if they showed more than 15% body weight loss for 3 consecutive days or 20% body weight loss during a single day.

When the time to endpoint (TTE) for each mouse was also calculated by the following equation:

$$TTE = \frac{log 10 ( endpoint volume ) - b}{m}$$

Where TTE is expressed in days, endpoint volume is in mm³, b is the intercept, and m is the slope of the line obtained by linear regression of a log-transformed tumor data set. This value was used to determined % tumor growth delay (% TGD), defined as the increase in median TTE for a treatment group compared to the control group expressed in days, or as a percentage of the median TTE of the control group.

Results:

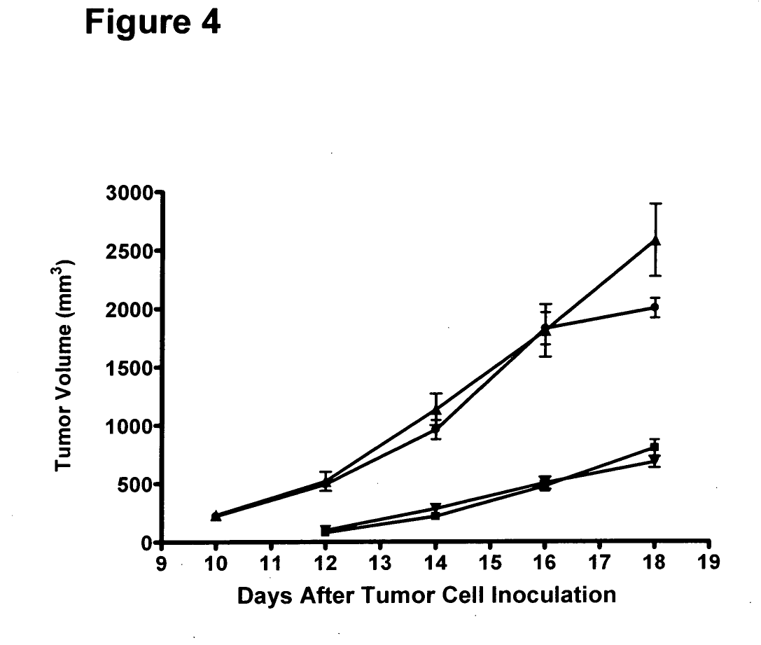

Compound 1 was administered following three different routes, SC, IP or IV, at different concentrations depending on the route of administration. Maximum body weight loss of 15% was observed on Day 13 for the IP group receiving 20 mg/kg (Q1D×8) followed by 10 mg/kg (Q1D×7) and 11% for the SC group receiving 30 mg/kg (Q1D×8) followed by 15 mg/kg (Q1D×7). No significant body weight loss was observed for the IV group. The effect of the different treatment routes on tumor growth inhibition was analyzed at Day 18. The efficacy data (FIG. 4) showed that daily bolus administrations of Compound 1-containing Formulation D11 either IP or SC resulted in significant antitumor efficacy in this tumor model, resulting in % TGI of 66% and 60% (P<0.0001). No significant difference in tumor volume relative to the vehicle control was noted for intravenous (IV) bolus administration of Formulation D11 at 100 mg/kg (Q1D×5) over 2 weeks. These data suggesting that Compound 1 efficacy would be correlated with prolonged exposure (as for SC and IP bolus administrations). Continuous intravenous infusion using the same formulation would be an alternative to the intravenous bolus administration.

B. Human U-87 MG Glioblastoma Mice Model

The human U-87 MG (ATCC® no. HTB-14™) glioblastoma antitumor efficacy study was performed at INSERM U318 (Grenoble, France). The U-87MG cell line is derived from a brain glioblastoma of a 44-year-old Caucasian female. On each dosing day, Compound 1 stock solutions (24 and 40 mg/mL in bulk Formulation B11) were diluted with sterile 5% dextrose in water (D5W) to prepare a dosing solution of 6 mg/mL of Compound 1 in ready-to-use Formulation D11 (5% ethanol, 5% PEG400, 15% polysorbate 80, and 75% D5W).

For the human glioblastoma antitumor efficacy study, female athymic (nu/nu) nude mice (6-7 weeks of age) were inoculated SC with 5×10⁶ U-87MG cells (day 0). Tumor bearing animals were randomized (10 per group) when tumors were palpable (day 24). Group 1 (control group) received drug-free vehicle (5% ethanol, 5% PEG-400, 15% polysorbate 80, and 75% D5W) SC (5 mL/kg), once daily q1d×15. Group 2 received Compound 1 (6 mg/mL) SC at 30 mg/kg, q1d×5 over 2 weeks (days 24-28 and 32-35). Group 3 (positive control group) received temozolomide PO at 150 mg/kg, q4d×3. Each animal was euthanized when its tumor reached the predetermined endpoint size (˜2,500 mm³) or at the end of the study (D40). Tumor growth inhibition (TGI) was calculated on day 34 post tumor cell inoculation, at which time some animals from the vehicle control group had to be sacrificed due to tumor burden.

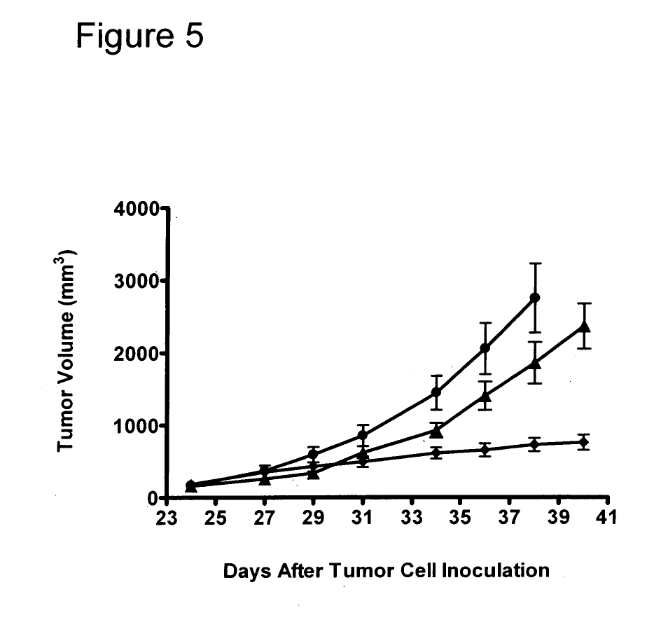

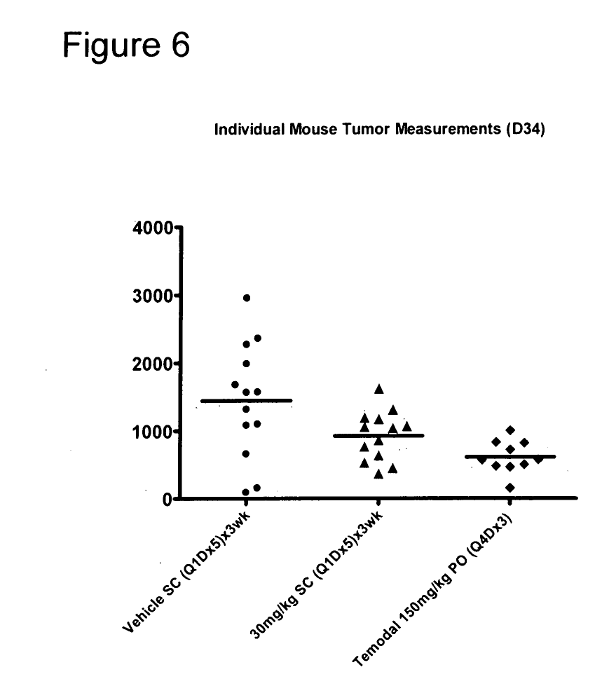

Compound 1 had demonstrated in vitro activity in this cell line with an IC₅₀ of 10.9 μM. Compound 1 antitumor activity in this model was tested by SC bolus injection (FIG. 5). The dose regimen was well tolerated with no significant body weight loss observed throughout the study. TGI was calculated at day 34, time at which some animals from the vehicle control group had to be sacrificed due to tumor burden. Moderate antitumor efficacy (% TGI=36%; P=0.05) was observed when Compound 1 was administered on a daily basis (FIG. 6).

C. Human PC3 Prostate Cancer Mice Model

The anticancer activity of Compound 1 was tested in a human PC3 prostate model in mice, using formulation D11; at 6, 9 and 10 mg/mL concentrations (see Table 7). HRLN male nude mice (8 weeks of age) were implanted with 1 mm³ PC3 tumor fragments subcutaneously (sc) in the right flank. Animals were randomized (ten per group) when tumors reach an average size of 80-120 mg and treatment began according to the table below.

TABLE 7
Dosing Schedules for Groups 1 to 6

| | | | | Concentration | Volume | | |
|---|---|---|---|---|---|---|---|
| Gr. | N | Agent | Dose (mg/kg) | (mg/mL) | (mL/kg) | Route | Schedule |
| 1 | 10 | Cyclophosphamide | 90 | 9 | 10 | IP | qd × 5 |
| 2 | 10 | D5W | — | — | 5 | SC | 5/2/5/2/5 |
| 3 | 10 | Compound 1 | 30 | 6 | 5 | SC | 5/2/5/2/5 |
| 4 | 10 | Compound 1 | 50 | 10 | 5 | SC | q3 d × 7 |
| 5 | 10 | Compound 1 | 30 | 6 | 5 | IP | q3 d × 7 |
| 6 | 10 | Compound 1 | 100 | 10 | 10 | IV | 5/2/5/2/5 |

Tumor measurements were taken twice weekly using callipers and were converted to tumor mass (in milligrams) using the formula: with² (mm)×length (mm)×0.52. Body weights were also recorded twice weekly. Statistical analysis was done using the unpaired two-tailed Student's t test.

% T/C was calculated at day 38 once animals in the control group had to be sacrificed due to antitumor burden. Intravenous treatment did not result in activity (likely due to short half-life and lack of sustaining therapeutically effective drug levels). On the other hand, subcutaneous administration at 30 mg/kg given from days 1 to 5, 8 to 12 and 15 to 19, or at 50 mg/kg every three days×7 (days 1, 4, 7, 10, 13, 16 and 19) where we maintain drug levels at therapeutically effective drug concentrations for over 8 hours resulted in significant antitumor activity with % T/C of 25.5% and 14.6%, respectively (P<0.0001).

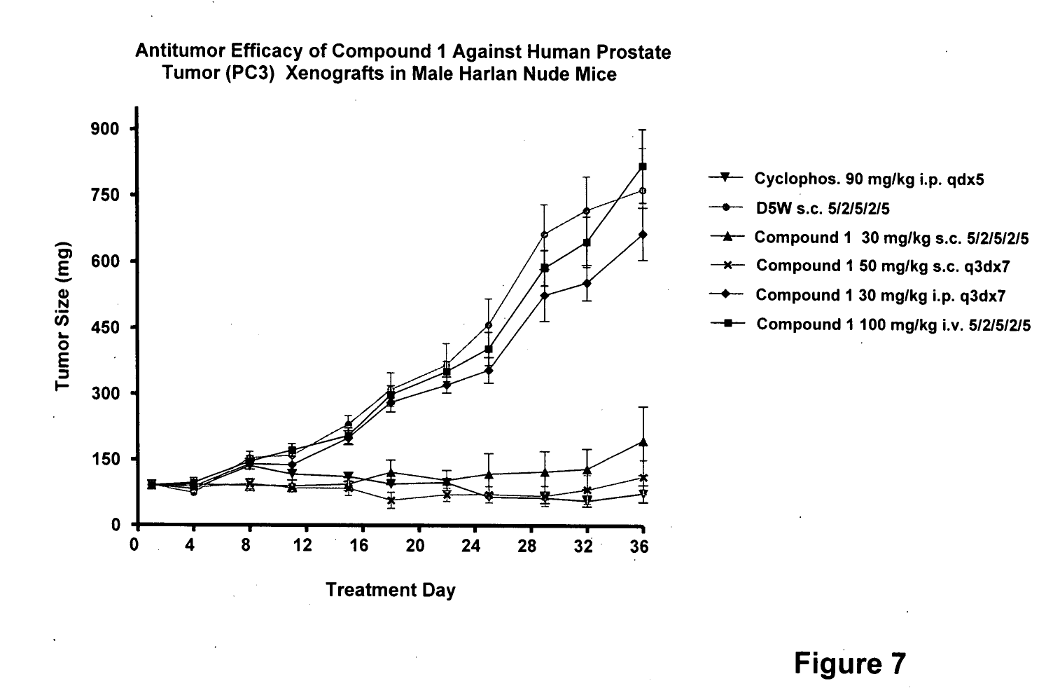

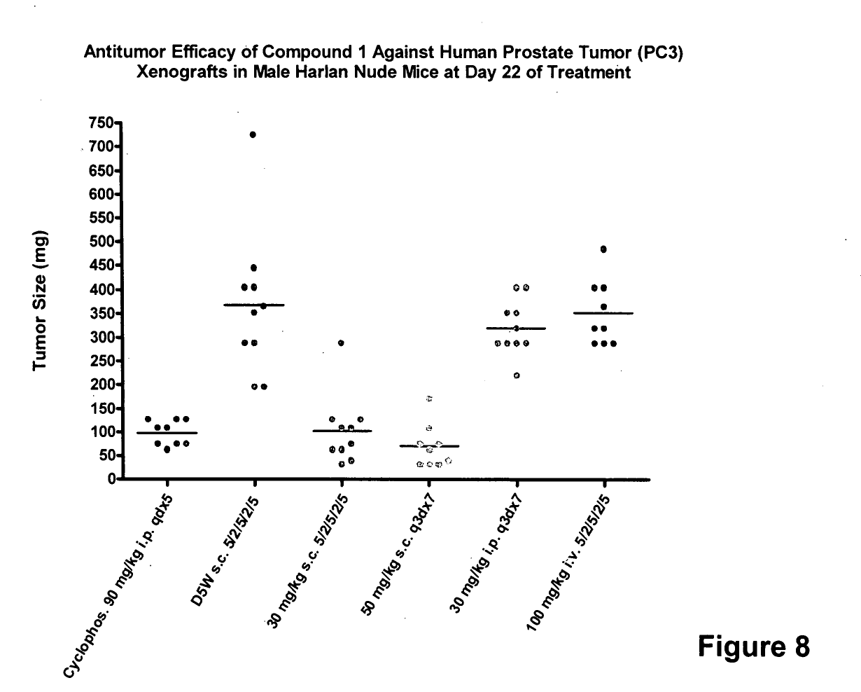

FIG. 7 shows antitumor efficacy results of Compound 1 in Formulation D11 against human prostate tumor xenografts. FIG. 8 shows antitumor efficacy results on individual animals on the 22^{nd} day of treatment.

D. Human MDA-MB-231 Breast Cancer Mice Model

The antitumor activity of Compound 1 was further tested in a human MD-MB-231 breast cancer model in mice, using formulation D11 at 6 and 10 mg/mL concentrations (see Table 8). HRLN female nude mice (8 weeks of age) were treated with 5×10⁶ MDA-MB-231 tumor cells (sc) in the right flank. Animals were randomized (ten per group) when tumors reach an average size of 80-120 mg and treatment began according to the table below.

TABLE 8
Dosing Schedules for Groups 1 to 8

| | | | | Concen- | | | |
|---|---|---|---|---|---|---|---|
| | | | Dose | tration | Volume | | |
| Gr | N | Agent | (mg/kg) | (mg/mL) | (mL/kg) | Route | Schedule |
| 1 | 10 | D5W | — | — | 10 | IV | 5/2/5/2/5 |
| 2 | 10 | paclitaxel | 30 | — | | IV | qod × 5 |
| 3 | 10 | Vehicle | — | — | 5 | SC | qd × 21 |
| 4 | 10 | Compound 1 | 100 | 10 | 10 | IV | 5/2/5/2/5 |
| 5 | 10 | Compound 1 | 30 | 6 | 5 | SC | 5/2/5/2/5 |
| 6 | 10 | Compound 1 | 20 | 6 | 3.3 | SC | qd × 21 |
| 7 | 10 | Compound 1 | 50 | 10 | 5 | SC | q3 d × 7 |
| 8 | 10 | Compound 1 | 30 | 6 | 5 | IP | q3 d × 7 |

Tumor measurements were taken twice weekly using calipers and were converted to tumor mass (in milligrams) using the formula: with² (mm)×length (mm)×0.52. Body weights were also recorded twice weekly. Statistical analysis was done using the unpaired two-tailed Student's t test.

% T/C was calculated at day 21 once animals in the control group had to be sacrificed due to tumor burden. Intravenous treatment did not result in activity (likely due to short half-life and lack of sustaining therapeutically effective drug levels). On the other hand, subcutaneous administration at 20 mg/kg given everyday for 21 days or at 30 mg/kg given from days 1 to 5, 8 to 12 resulted in significant antitumor activity with % T/Cs of 40% and 35% respectively; P<0.0001). Subcutaneous or intraperitoneal administration at 50 and 30 mg/kg respectively every three days×7 (days 1, 4, 7, 10, 13, 16 and 19) were also effective giving moderate but statistically significant T/C values of 68% (P=0.0019) and 58% (P=0.0007).

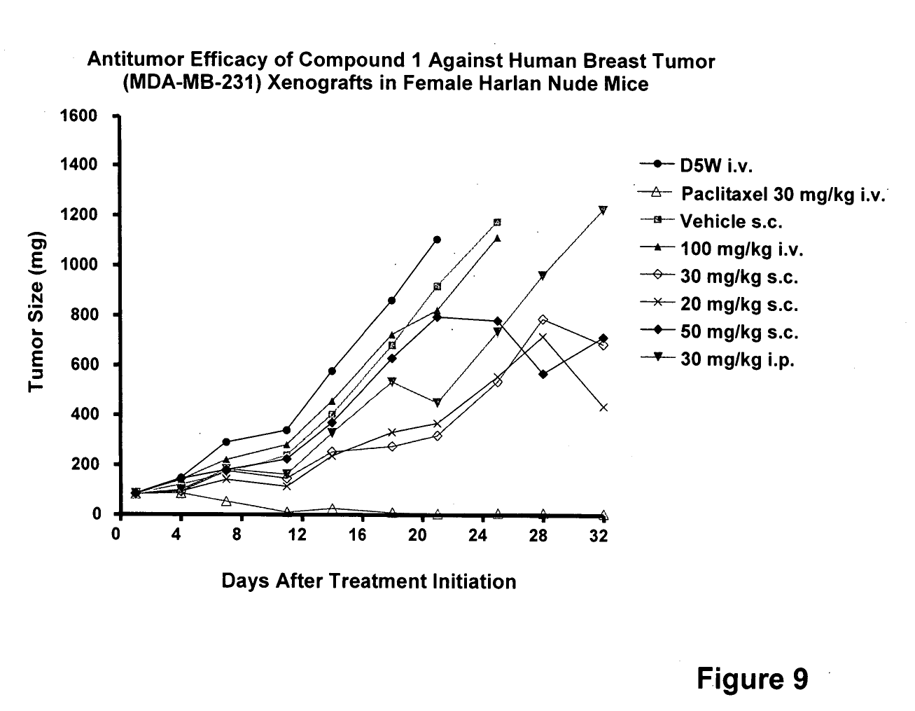

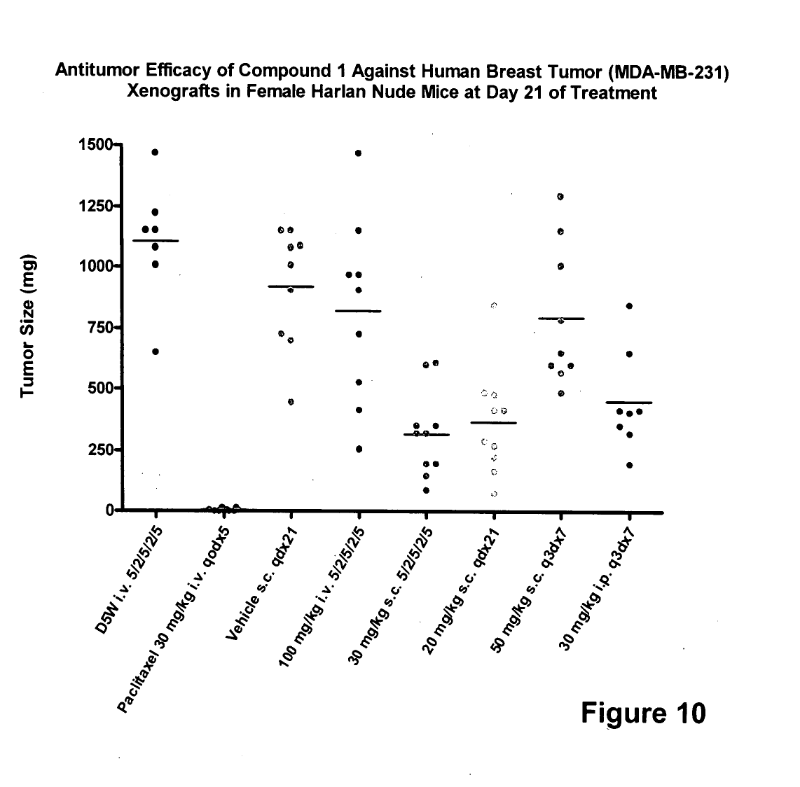

FIG. 9 shows antitumor efficacy results of Compound 1 in Formulation D11 against human breast tumor xenografts. FIG. 10 shows antitumor efficacy results on the 21^{st} day of treatment.

### Example 11

### In Vivo CIV Administration of Formulation D11

A. In Vivo Pharmacokinetics of Formulation DII given CIV in Rats:

Sprague-Dawley rats received an intravenous continuous infusion over 14 days of Compound 1 at 25 mg/kg/day, 50 mg/kg/day, or 75 mg/kg/day at a rate of 2 mL/kg/h for 14 consecutive days (Formulation D11). Blood was collected from the jugular vein in tubes containing K₂ EDTA from 3 rats/sex/group at the following time points: 2, 6, and 12 hours after the start of dosing on Day 1, on Day 2 at 6 hours (approximately 30 hours after the start of dosing), on Days 6 and 10 at 6 hours, and on Day 15, 1 hour prior the end of dosing, and then at 5 min, 15 min, 30 min, 1 h, and 2 h after the end of dosing.

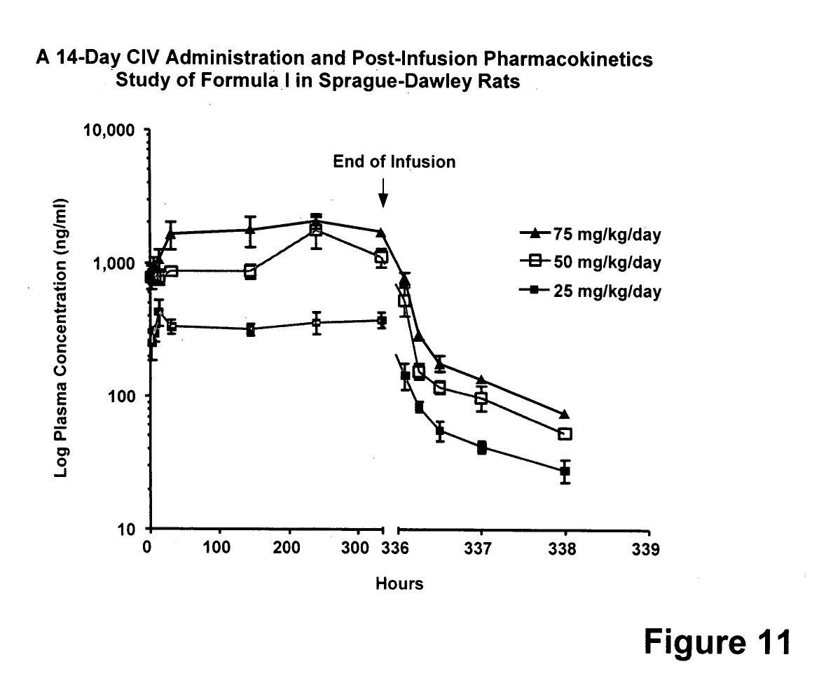

Results from this 14-day IV continuous infusion of Compound 1 are shown in Table 9 and FIG. 11. For the groups that received 25 mg/kg/day or 75 mg/kg/day, steady-state Compound 1 plasma concentrations were observed throughout the 14-day CIV infusion, with steady-state plasma concentrations of 347 ng/mL (˜0.8 μM) and 1,796 ng/mL (˜3.9 μM), respectively. For the mid dose group of 50 mg/kg/day, Compound 1 plasma concentration was unusually high on Day 10 (1,753 ng/mL or ˜3.8 μM) and decreased back to the steady-state level at Day 14 as measured during prior measurements (1,150 ng/mL or ˜2.5 μM), suggesting possible analytical or biological variability. Mean steady-state plasma concentrations in the 50 mg/kg/day and 75 mg/kg/day groups exceeded the therapeutic threshold of 2 μM defined in the in vivo antitumor activity experiments throughout the 14-day infusion period, with concentrations of ˜2.5 μM and ˜3.9 μM, respectively. AUCs for the different groups increased with increasing dose level, but this increase was slightly greater than dose-proportional with an AUC of 116,418 ng/mL*h for the 25 mg/kg/day group, 396,134 ng/mL*h for the 50 mg/kg/day group, and finally 597,378 ng/mL*h for the 75 mg/kg/day group. When infusion of Compound 1 in the different groups was terminated, rapid elimination of Compound 1 from plasma was observed in all groups, showing that Compound 1 is rapidly cleared from plasma. At 2 hours after the end of infusion of Compound 1, the mean concentration of Compound 1 had declined to 28 ng/mL in the low dose group (25 mg/kg/day), 53 ng/mL in the mid dose group (50 mg/kg/day), and to 75 ng/mL for the high dose group (75 mg/kg/day). The T_{1/2}z for Compound 1 varied between 1.2 and 1.6 h for the different dosage groups.

TABLE 9
PK Results in rats from a Compound 1 14-day CIV infusion

| Dose | | | | | | |
|---|---|---|---|---|---|---|
| (mg/ | Css | AUC_{α} | CL | Vss | Vz | T_{1/2}z |
| kg/day) | (ng/mL)^{a} | (ng/mL * h) | (L/h/kg) | (L/kg) | (L/kg) | (h)^{b} |
| 25 | 347 | 116,418 | 3.0 | 15.8 | 6.8 | 1.6 |
| 50 | 1,150 | 396,134 | 1.8 | 38.8 | 3.1 | 1.2 |
| 75 | 1,796 | 597,378 | 1.8 | 15.4 | 3.1 | 1.2 |

^{a}Average of plasma concentration between 30 h and 14 days.
^{b}Calculated at the end of treatment

In summary, the results showed that steady-state Compound 1 plasma concentrations above the therapeutic threshold of 2 μM were obtained with a 14-day IV continuous infusion of Compound 1 in rats at doses of 50 and 75 mg/kg/day. When dosing of Compound 1 was terminated after 14 days, the drug was rapidly eliminated from the plasma of rats for all dosing groups.

B. In Vivo Pharmacokinetics of Formulation D11 given CIV in Monkeys:

Cynomolgus monkeys received continuous IV infusion over 14 days of Compound 1 at 5 mg/kg/day, 15 mg/kg/day, or 30 mg/kg/day. The drug was infused intravenously (24 hours/day) into the femoral vein at a dose rate of 2 ml/kg/hour for 14 consecutive days. Blood samples were removed from each monkey on Days 1, 2, 6, 10, and 15 of the treatment period. Monkeys were bled by venipuncture and samples were collected into tubes containing K₂EDTA. On Day 1, samples were collected at 2, 6, and 12 hours after initiation of treatment. Additional samples were collected at 30 hours after the start of infusion (Day 2). On Days 6 and 10, samples were collected at approximately 6 hours after the bag changes. At the end of the 14 days of infusion, on Day 15, samples were collected at 1 hour prior to cessation of dose administration, and at 5 min, 30 min, 1 h, 2 h, 4 h, 8 h, and 24 h following cessation of dose administration.

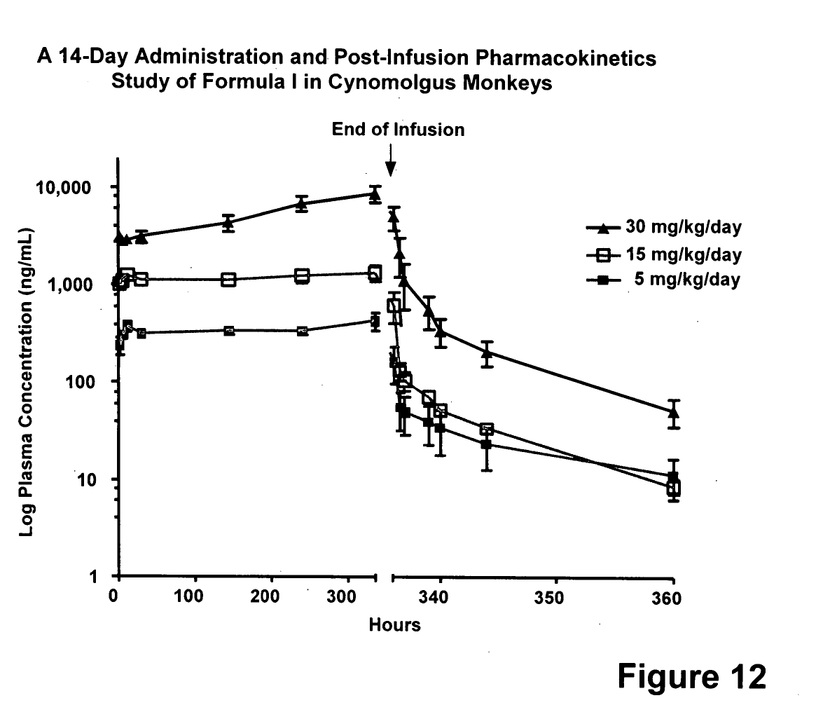

Results from this 14-day IV continuous infusion of Compound 1 are shown in Table 10 and FIG. 12. For the groups that received a 5 mg/kg/day dose or 15 mg/kg/day, steady-state Compound 1 plasma concentrations were observed throughout the 14-day CIV infusion, with mean steady-state plasma concentrations (between 30 h and 14 days) of 358 ng/mL (˜0.8 μM) and 1,173 ng/mL (˜2.5 μM), respectively. For the high dose group of 30 mg/kg/day, Compound 1 plasma concentration increased throughout the 14-day infusion period from 2,814 ng/mL (˜6.1 μM) at Day 1 to 4,354 ng/mL (˜9.4 μM) at Day 6, to 6,855 ng/mL (˜15 μM) by Day 10, and to 8,561 ng/mL (˜18.5 μM) by day 15. Plasma concentrations in the 15 mg/kg/day and the 30 mg/kg/day groups exceeded the therapeutic threshold observed in the in vivo antitumor activity experiments throughout the 14-day infusion period. AUCs for the different groups increased approximately proportionally to the dose received between the low and middle dose groups, with a mean AUC of 119,018 ng/mL*h for the 5 mg/kg/day group, 400,116 ng/mL*h for the 15 mg/kg/day group (3.4-fold increase between the groups, which is proportional to the 3-fold increase in dose level). However, the AUC value for the high dose group (30 mg/kg/day) was markedly greater, i.e. 1,874,950 ng/mL*h, which is 4.7-fold higher than that of the middle dose group, despite the 2-fold increase in dose level. When infusion of Compound 1 in the different groups was terminated, rapid elimination of Compound 1 from plasma was observed in all groups. The T_{1/2}z for Compound 1 varied between 8.1 and 11.5 h for the different dosage groups.

TABLE 10
PK Results in monkeys from a Compound 1 14-day CIV infusion

| Dose | Css | AUC_{α} | CL | Vss | Vz | T_{1/2}z |
|---|---|---|---|---|---|---|
| (mg/kg/day) | (ng/mL)^{a} | (ng/mL * h) | (L/h/kg) | (L/kg) | (L/kg) | (h)^{c} |
| 5 | 358^{b} (85) | 119,018 (26,690) | 0.61 (0.14) | 7.1 (3.9) | 10 (3) | 12 (3) |
| 15 | 1,173 (340) | 400,116 (126,140) | 0.56 (0.13) | 3.6 (2.0) | 6.8 (2.9) | 8.3 (2.2) |
| 30 | 6,283 (3,650) | 1,874,950 (945,067) | 0.27 (0.11) | 10.7 (6.2) | 3.2 (1.7) | 8.1 (1.0) |

^{a}Average of plasma concentration between 30 h and 14 days.
^{b}Values are Mean (SD).
^{c}Calculated at the end of treatment

In summary, the results showed that stable Compound 1 plasma concentrations above the therapeutic threshold of 2 μM are obtained with a 14-day IV continuous infusion of Compound 1 in monkeys. When dosing of Compound 1 was terminated after 14 days, the drug was rapidly eliminated from the plasma of monkeys for all dosing groups.

C. In Vivo Toxicity of Compound 1 in Rats and Monkeys:

When administered as a 14-day continuous intravenous infusion (as in b), no severe compound associated toxicity was observed in monkeys, and side effects, including inappetance and a moderate degree of regenerative anemia, were reversible. Diffuse vacuolization of hepatocytes and accumulation of foamy histiocytes (macrophages) in the spleen were observed in monkeys, which reflected clearance of the vehicle used. No degenerative changes were observed in any organs, including the infusion site, and there were no effect on body weight, ocular condition, electrocardiographic activity and other parameters assessed in the monkeys.

In the rats, a 14-day infusion (as in a) was associated with necrotization and inflammatory lesions at the site of infusion for all treated and control groups. The toxicity was due to the vehicle and was attributed to smaller size of infusion vessels, and concurrent catheter tract infection.

Single bolus intravenous administration showed an MTD of 85 mg/kg in healthy rats, and an MTD of about 35 mg/kg in monkeys.

Acute toxicity was also evaluated in a 24-hour CIV administration schedule in monkeys and doses of 35 mg/kg and 70 mg/kg, for a period of 24 hours (infusion rate of 2 mL/kg/hour), were both well tolerated.

### Example 12

### Simulation of the Pharmacokinetics of Compound 1 in Humans

An analysis was performed to derive allometric equations for Compound 1 pharmacokinetic parameters using Compound 1 plasma concentration-time data from three species, including mouse, rat, and monkey, and to estimate human pharmacokinetic parameters from those allometric equations.

Plasma concentrations of Compound 1 were obtained from mice, rats, and monkeys following intravenous injection or continuous infusion. Compound 1 pharmacokinetic parameters in mice, rats, and monkeys were estimated using population pharmacokinetic analysis, a function of the software program NONMEM™ (version 5). Typical population pharmacokinetic parameters for Compound 1 in humans were extrapolated from allometric equations that were derived from pharmacokinetic parameters estimated in the three animal species. Compound 1 plasma concentration-time profiles following 9-day or 14-day continuous infusion were simulated in a patient (weight, 70 kg; BSA, 1.8 m²) with a typical population clearance (mean CL), 50% higher clearance (mean CL+50%×mean CL), and 50% lower clearance (mean CL−50%×mean CL), respectively.

A two-compartment model with a first-order elimination from the central compartment adequately described Compound 1 plasma concentration-time profiles following intravenous bolus injection (30 mg/kg) in mice and rats, 7-day continuous infusion in rats (50 to 170 mg/kg/day), and 14-day continuous infusion in monkeys (5 to 30 mg/kg/day). The estimated population pharmacokinetic parameters of Compound 1 in mice, rats, and monkeys are presented in Table 11.

TABLE 11
Typical Population Pharmacokinetic Parameters in Mouse,
Rat, Monkey, and Estimated Parameters in Humans^{a}

| | WT | V1 | V2 | Q | Ke | CL | Vss | t_{1/2}, α | t_{1/2}, β |
|---|---|---|---|---|---|---|---|---|---|
| | (Kg) | (L/kg) | (L/kg) | (L/h/kg) | (h⁻¹) | (L/h/kg) | (L/kg) | (h) | (h) |
| Mouse | 0.022 | 0.176 | 0.225 | 0.0654 | 8.466 | 1.49 | 0.401 | 0.078 | 2.49 |
| (IV) | | | | | | | | | |
| Rat | 0.225 | 0.126 | 0.236 | 0.0696 | 8.651 | 1.09 | 0.362 | 0.075 | 2.50 |
| (IV + CIV) | | | | | | | | | |
| Monkey | 3.8 | 0.203 | 0.419 | 0.0398 | 2.172 | 0.441 | 0.622 | 0.308 | 8.05 |
| (CIV) | | | | | | | | | |
| Human | 70 | 0.198 | 0.559 | 0.032 | 1.192 | 0.236 | 0.757 | 0.509 | 13.8 |
| | WT | V1 | V2 | Q | | CL | Vss | | |
| | (Kg) | (L) | (L) | (L/h) | | (L/h) | (L) | | |
| Mouse | 0.022 | 0.004 | 0.005 | 0.001 | — | 0.032 | 0.009 | — | — |
| (IV) | | | | | | | | | |
| Rat | 0.225 | 0.028 | 0.053 | 0.016 | — | 0.245 | 0.081 | — | — |
| (IV + CIV) | | | | | | | | | |
| Monkey | 3.8 | 0.771 | 1.59 | 0.151 | — | 1.68 | 2.360 | — | — |
| (CIV) | | | | | | | | | |
| Human | 70 | 13.9 | 39.2 | 2.27 | — | 16.5 | 53.1 | — | — |

^{a}Population pharmacokinetic parameters for Compound 1 in humans were estimated from allometric equations derived from three species mouse, rat, and monkey.

A 14-day continuous infusion in monkeys resulted in mean steady-state plasma concentrations of 0.75, 2.57, and 14.07 μM at dose levels of 5, 15, and 30 mg/kg/day, respectively, and corresponding mean clearance values of 0.63, 0.57, and 0.23 Uh/kg, respectively. Application of a two-compartment model with Michaelis-Menten elimination better described the concentration data in monkeys than the linear model. Because the target concentration in humans is 2 μM, at which linear pharmacokinetics is assumed, all simulations for human plasma concentrations were performed based on a two-compartment model with linear first-order elimination.

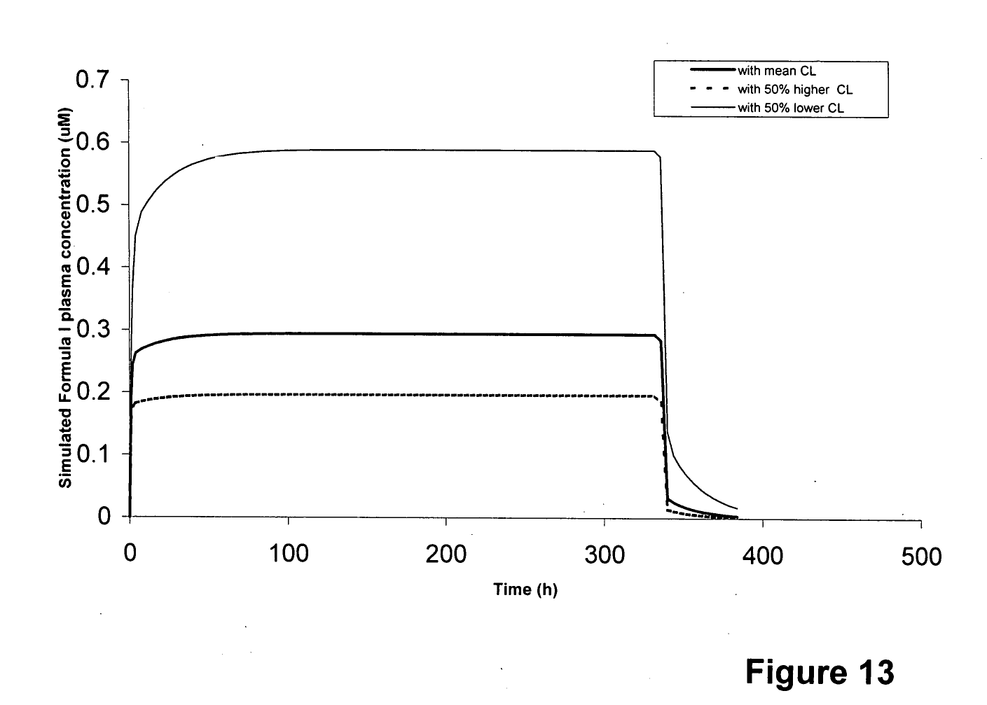

Allometric equations for the pharmacokinetic parameters clearance (CL), volume of distribution (V1 and V2), and inter-compartmental clearance (O) were derived. The population PK parameters of the compound of Compound 1 for humans were extrapolated from the allometric equations, and the estimated values are shown in Table 11. Simulated Compound 1 plasma concentration-time profiles in humans are shown in FIG. 13 and estimated end of infusion concentrations are provided in Table 12.

TABLE 12
Projected Compound 1 Steady-state Concentrations
Following CIV Infusion in Humans

| | | Estimated Compound 1 | | |
|---|---|---|---|---|
| | | Plasma Concentration^{a} (μM) | | |
| | Dose | Typical Population | 50% Higher | 50% Lower |
| | (mg/m²/day) | Clearance | Clearance | Clearance |
| CIV | 30 | 0.29 | 0.20 | 0.59 |
| (For 14 | 60 | 0.59 | 0.40 | 1.2 |
| days) | 120 | 1.2 | 0.79 | 2.4 |
| | 180 | 1.8 | 1.2 | 3.5 |

^{a}Compound 1 concentrations were estimated for a patient (70 kg, BSA 1.8 m²) with typical mean population pharmacokinetic parameters (CL, 0.236 L/h/kg; V1, 0.198 L/kg; V2, 0.559 L/kg; Q, 0.032 L/h/kg), a patient with 50% lower CL than the typical mean value (0.118 L/h/kg), and a patient with 50% higher CL than the typical mean value (0.354 L/h/kg).

From the simulation of a 14-day continuous infusion of Compound 1 at a dose of 30 mg/m²/day, the estimated steady-state plasma concentration, using parameters of an average patient, was 0.29 μM (Table 12). We have observed in the pharmacokinetic profiling of Compound 1 in monkeys that, in a 14-day continuous IV infusion, at doses of 5 mg/kg/day and 15 mg/kg/day, steady-state Compound 1 plasma concentrations were observed throughout the 14-day CIV infusion. It can thus be anticipated that dosing of Compound 1 at 180 mg/m²/day (4.5 mg/kg/day) in humans will produce steady-state plasma concentrations of Compound 1 during a continuous IV infusion over 14 days.

### Example 13

### Administration of Formulation B10 to Humans

Bulk Formulation B10 as described above is used for administration to humans for the treatment of cancer. The bulk formulation is reconstituted in sterile 0.9% saline prior to patient administration. Bulk formulation vials are provided with a drug reconstitution kit consisting of a sterile 60 mL pre-filled syringe containing 52 mL of 0.9% saline, infusion bag, and administration set (with pump connector) and extension set. The extension set comprises an anti-siphon valve and a sterile 0.2 micron in-line filter. The vial content is diluted with 52 mL of sterile 0.9% saline with the aid of a pre-filled syringe. This overfill ensures that there is a minimal extractable premix volume of 59 mL containing 4.48 mg/mL of Compound 1, which corresponds to 265 mg/vial. The dosing formulation is isotonic at this drug concentration in 0.9% saline.

Depending on the dose to administer, the dosing formulation is then transferred to a 250-mL, 500-mL, or 1-L EVA or PP infusion bag. The infusion bag is connected to a CADD Prizm VIP 6101 model pump for continuous 24-hour infusion. The daily dose is adjusted with the flow rate of the pump, which is programmed and locked by the pharmacist. Patient is monitored for adverse side effects and efficacy of the treatment.

For example, a 180 mg/m² daily dose is given during a period of 14 days to a human patient having a 1.8 m² body surface area. The patient is administered a daily volume of about 72.34 mL (324.1 mg of drug), for a total of 1012.8 mL (4537.4 mg of drug) of the reconstituted formulation above at a flow rate adjusted to about 3.014 mL/h. The 14-day infusion is given in two 7-day infusions, i.e. changing infusion bag after 7 days, each bag administering a total volume of about 506.4 mL. The patient is then allowed to rest for 7 days. One or more additional 14-day infusion treatments are given in the same manner, with or without adjustment of the dosage, depending on response and adverse side effects.

All patents, patent applications, and published references cited herein are hereby incorporated by reference in their entirety. In case of conflict, the present specification, including definitions, will control. While this invention has been particularly shown and described with reference to preferred embodiments thereof, it will be understood by those skilled in the art that various changes in form and details may be made therein without departing from the scope of the invention encompassed by the appended claims.

1. A pharmaceutical formulation comprising an active ingredient, together with a pharmaceutically acceptable vehicle comprising a pharmaceutically acceptable surfactant, wherein said active ingredient is a compound of Formula I, or a pharmaceutically acceptable salt, solvate or prodrug thereof: 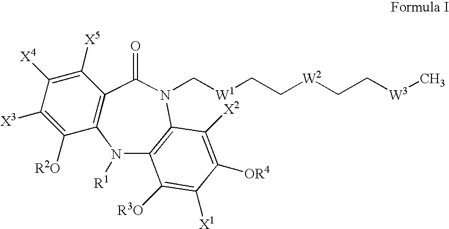
wherein,
W¹, W² and W³ are each independently selected from 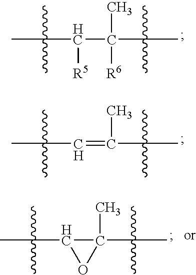
the chain from the tricycle terminates at W³, W² or W¹ with W³, W² or W¹ respectively being either —CH═O, —CH(OC₁₋₆alkyl)₂, —CH₂OH, —CH₂OC₁₋₆alkyl or C(O)OR⁷;
R¹ is selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R², R³, and R⁴ are each independently selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl, C₃₋₁₀heterocycloalkyl, C(O)H, C(O)C₁₋₁₀alkyl, C(O)C₂₋₁₀alkenyl, C(O)C₂₋₁₀alkynyl, C(O)C₆₋₁₀aryl, C(O)C₅₋₁₀heteroaryl, C(O)C₃₋₁₀cycloalkyl; C(O)C₃₋₁₀heterocycloalkyl or a C-coupled amino acid;
R⁵ and R⁶ are each independently selected from H, OH, OC₁₋₆alkyl, OC(O)C₁₋₆alkyl, NH₂, NHC₁₋₆alkyl, N(C₁₋₆alkyl)₂, NHC(O)C₁₋₆alkyl;
R⁷ is selected from H, C₁₋₁₀alkyl, C₂₋₁₀alkenyl, C₂₋₁₀alkynyl, C₆₋₁₀aryl, C₅₋₁₀heteroaryl, C₃₋₁₀cycloalkyl and C₃₋₁₀heterocycloalkyl;
X¹, X², X³, X⁴ and X⁵ are each H; or
one of X¹, X², X³, X⁴ or X⁵ is halogen and the remaining ones are H; and wherein, when any of R¹, R², R³, R⁴, R⁵, R⁶ and R⁷ comprises an alkyl, alkenyl, alkynyl, aryl, heteroaryl, cycloalkyl, or heterocycloalkyl group, then the alkyl, alkenyl, alkynyl, aryl, heteroaryl, cycloalkyl, or heterocycloalkyl group is optionally substituted with substituents selected from acyl, amino, acylamino, acyloxy, carboalkoxy, carboxy, carboxyamido, cyano, halo, hydroxyl, nitro, thio, C₁₋₆alkyl, C₂₋₇alkenyl, C₂₋₇alkynyl, C₃₋₁₀cycloalkyl, C₃₋₁₀heterocycloalkyl, C₆₋₁₀aryl, C₅₋₁₀heteroaryl, alkoxy, aryloxy, sulfinyl, sulfonyl, oxo, guanidino and formyl.

2. The pharmaceutical formulation of claim 1, wherein said active ingredient is selected from the group consisting of Compounds 1 to 130: 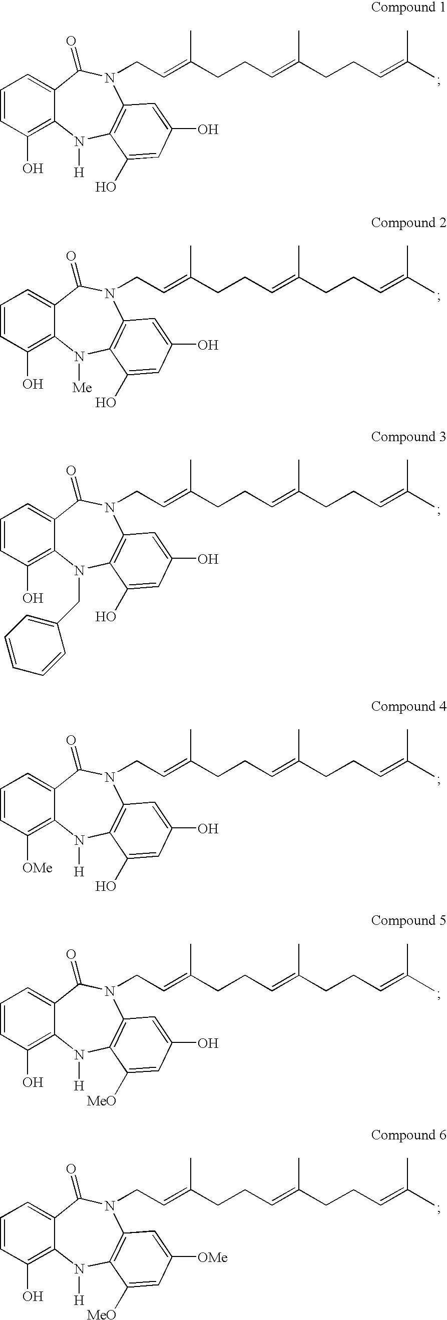 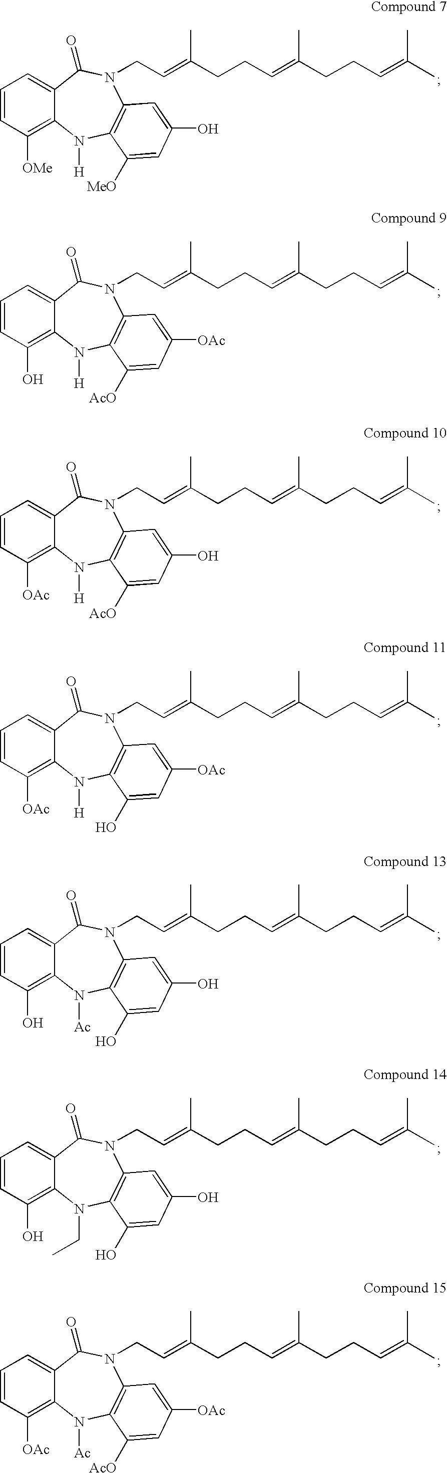 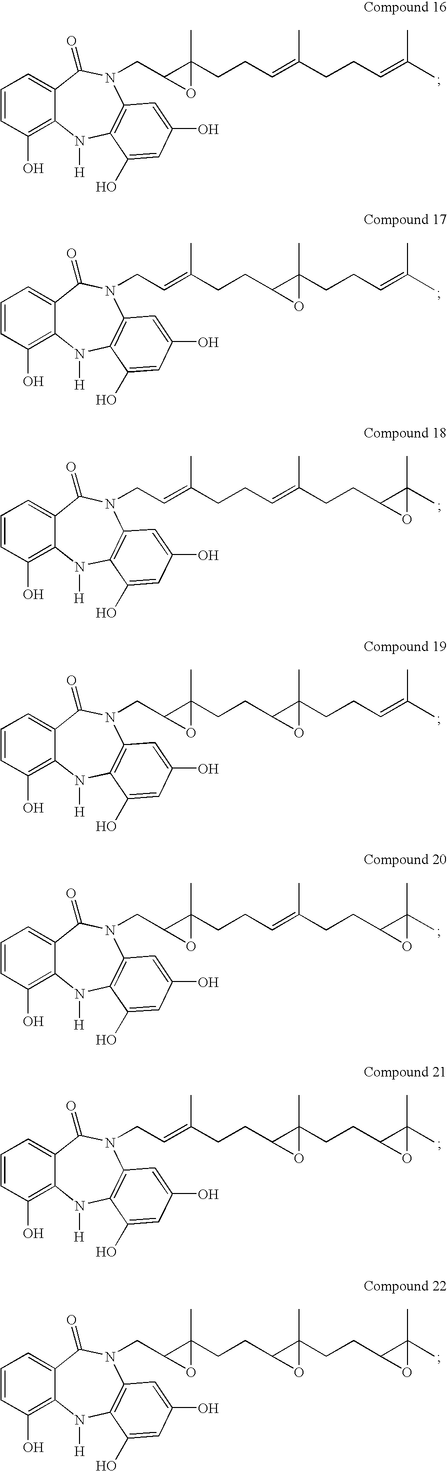 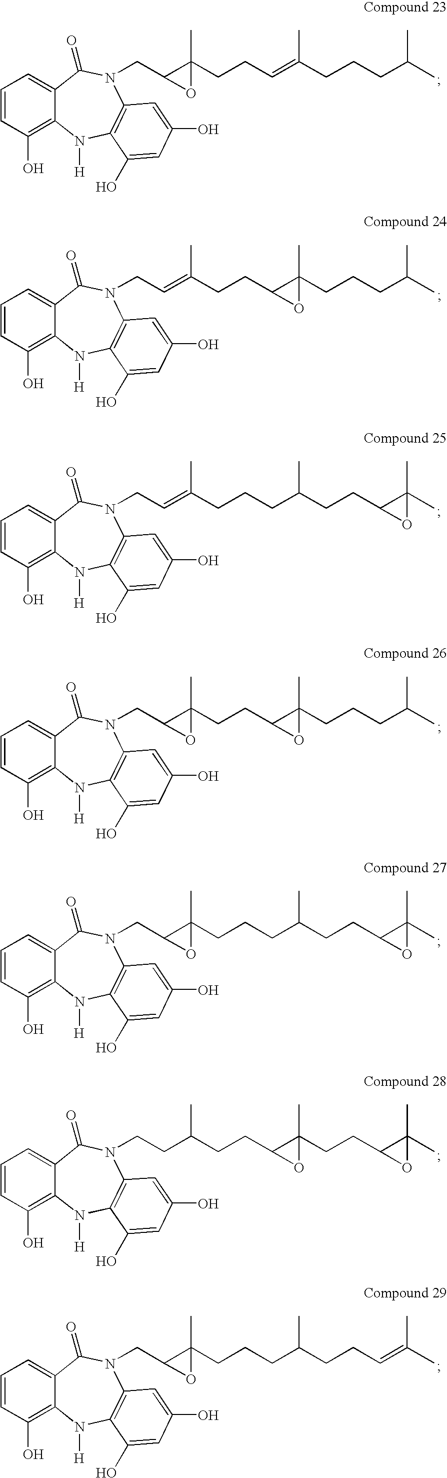 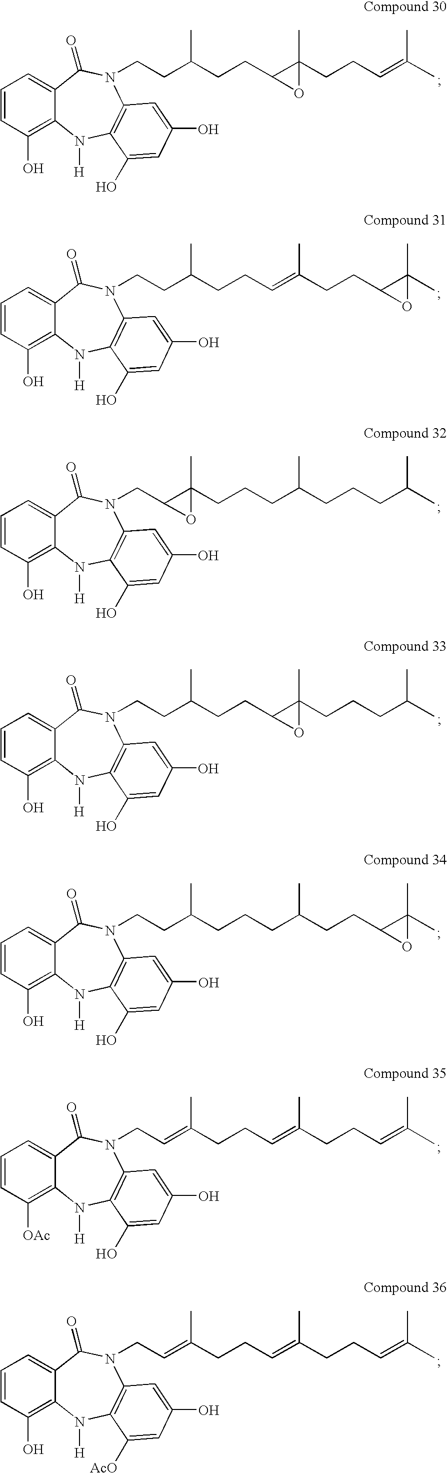 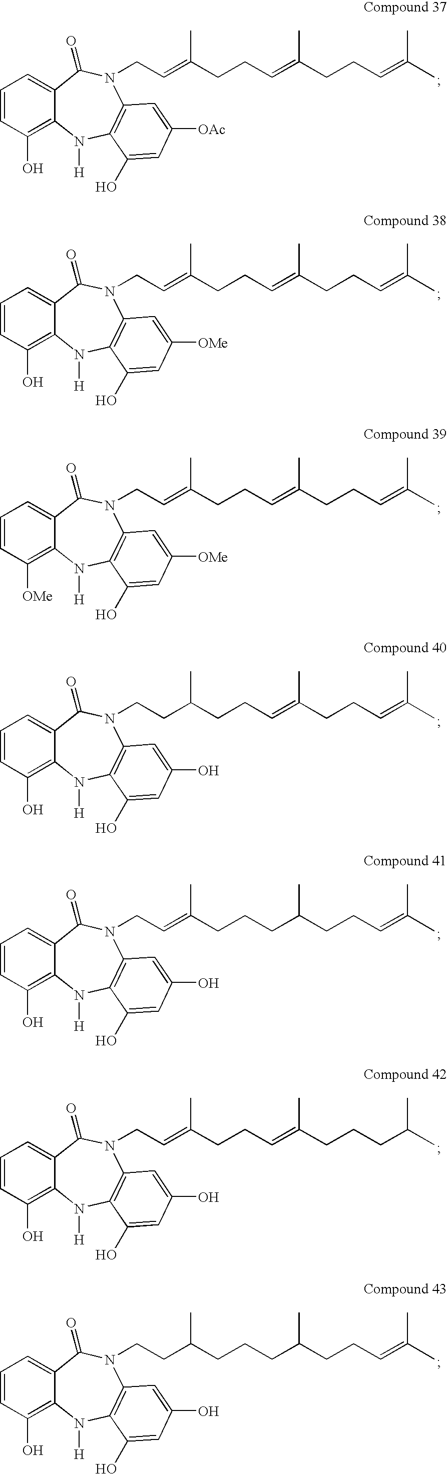 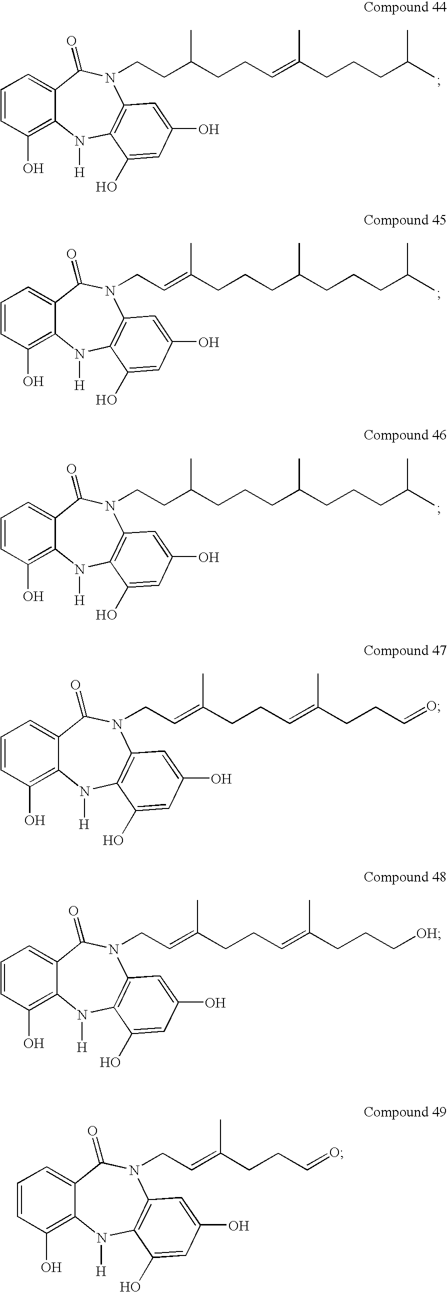 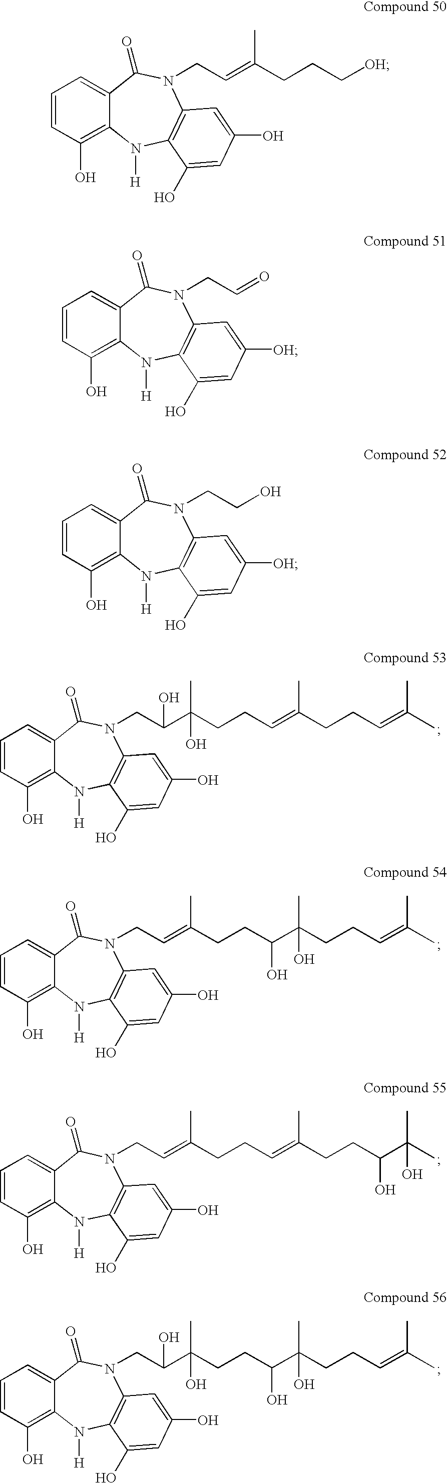 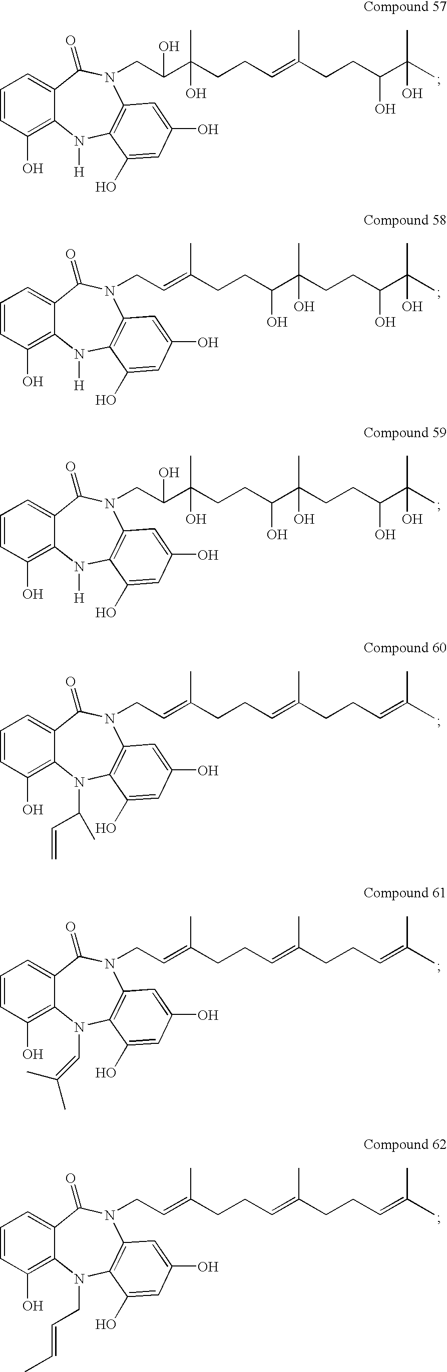 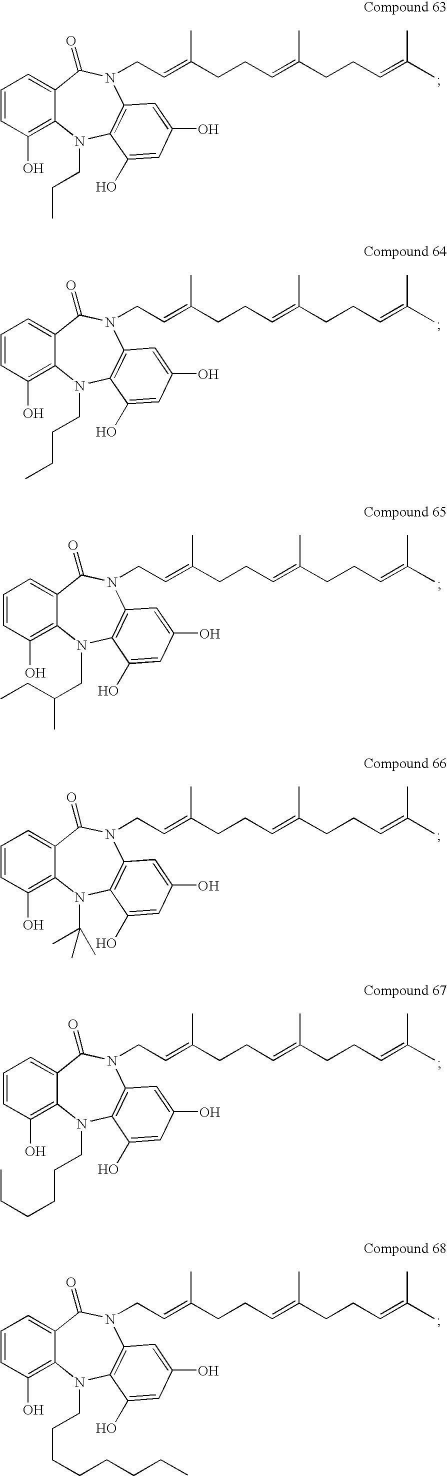 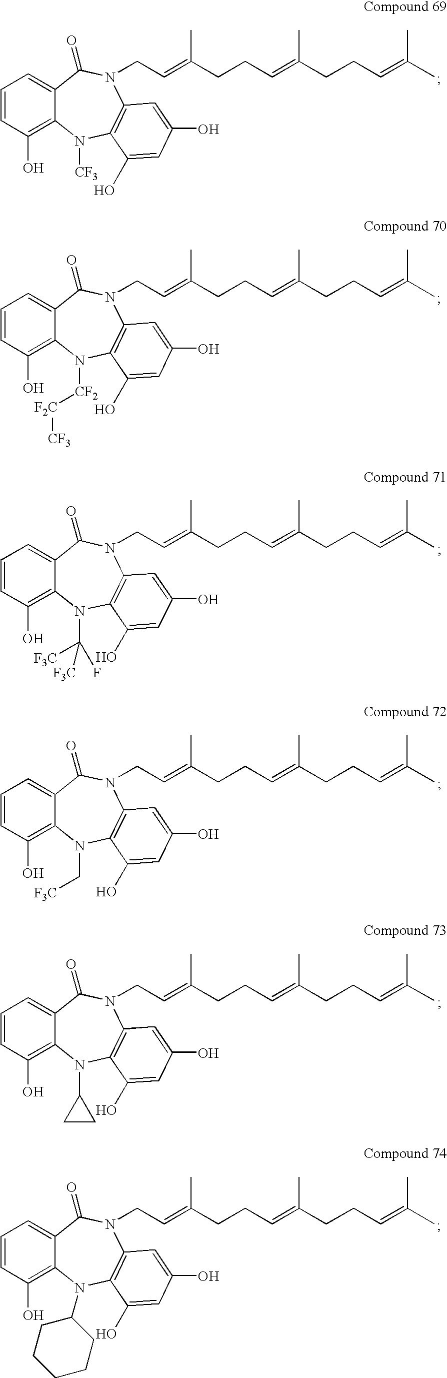 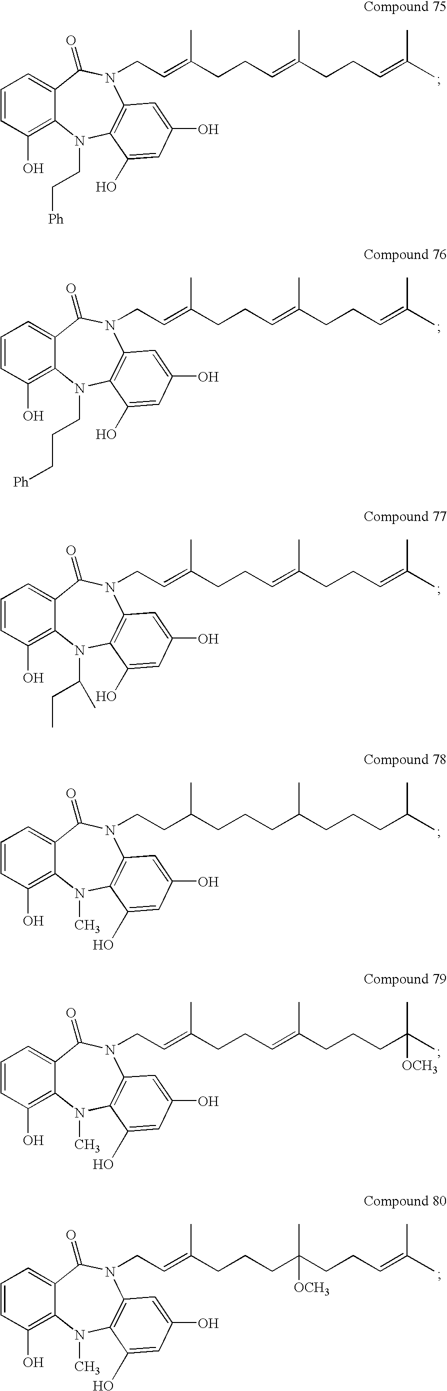 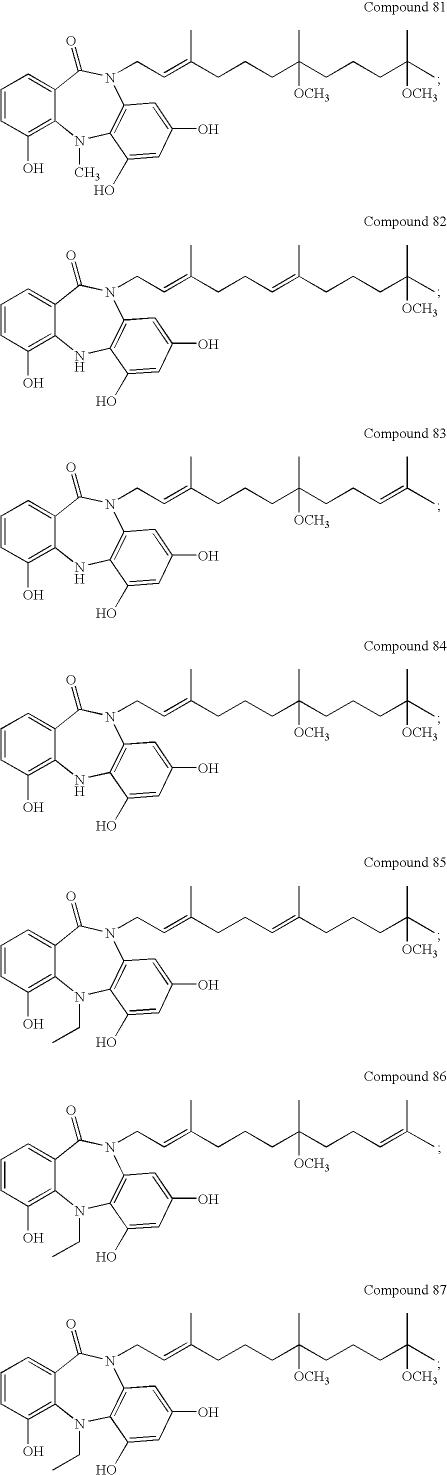 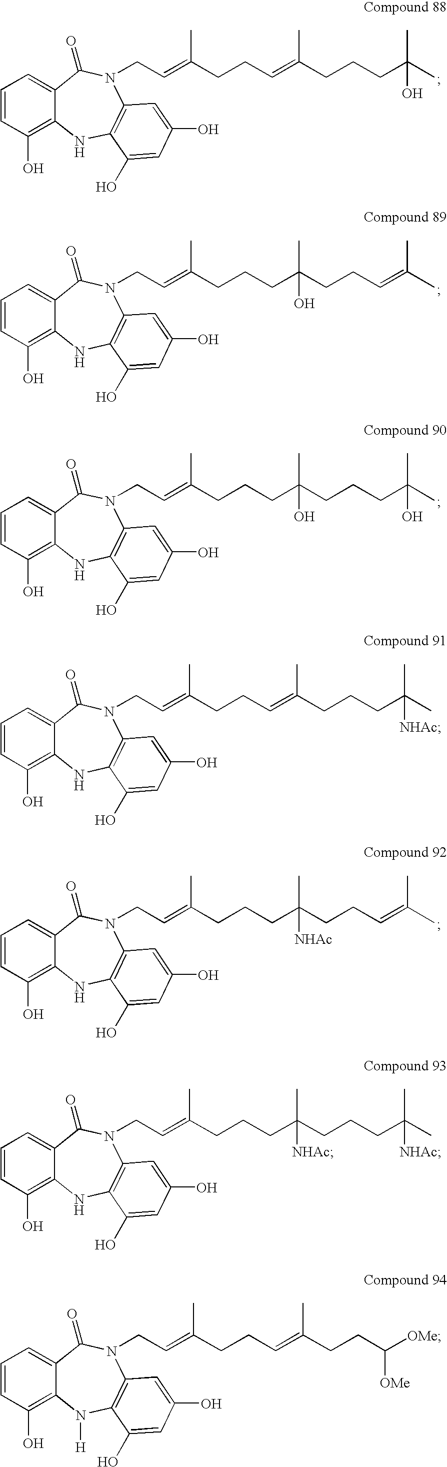 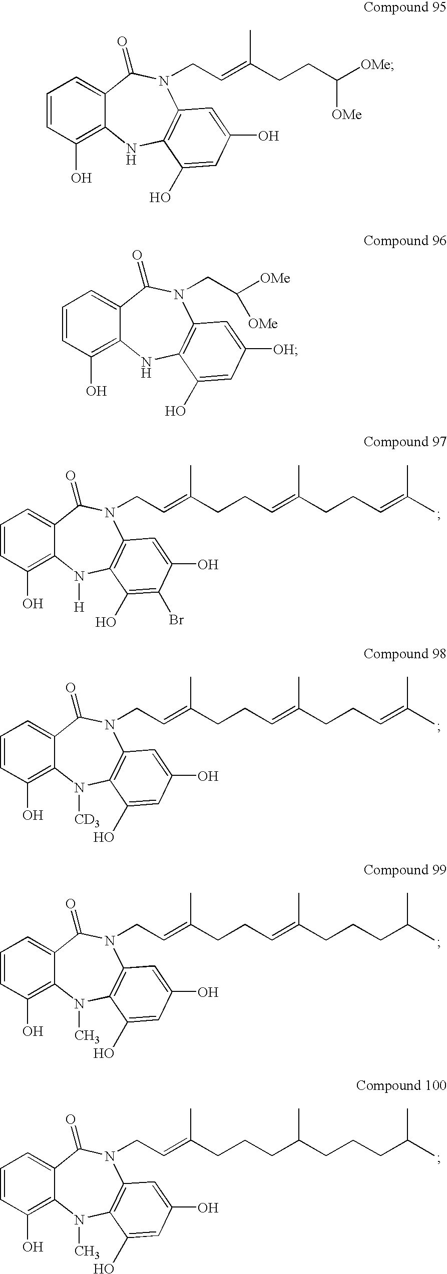 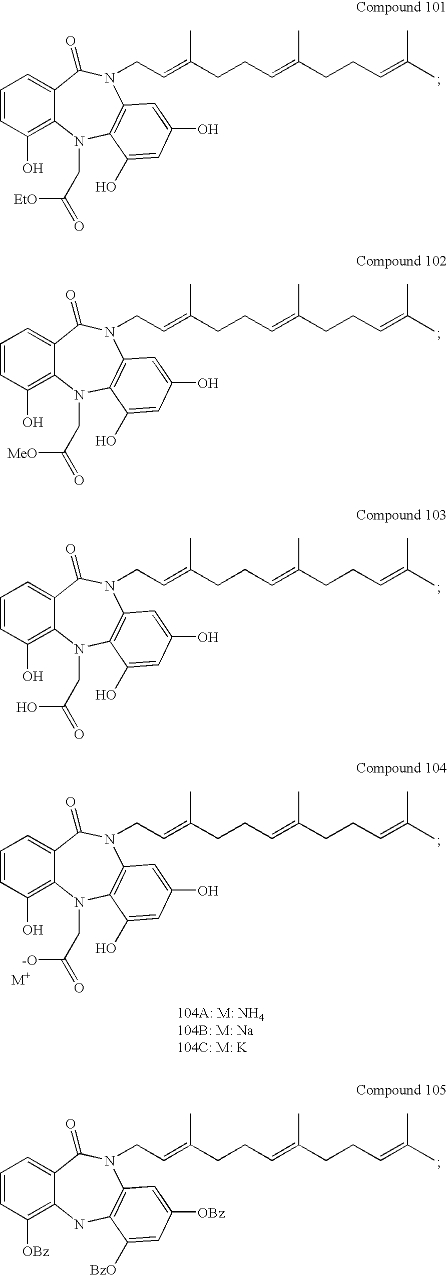 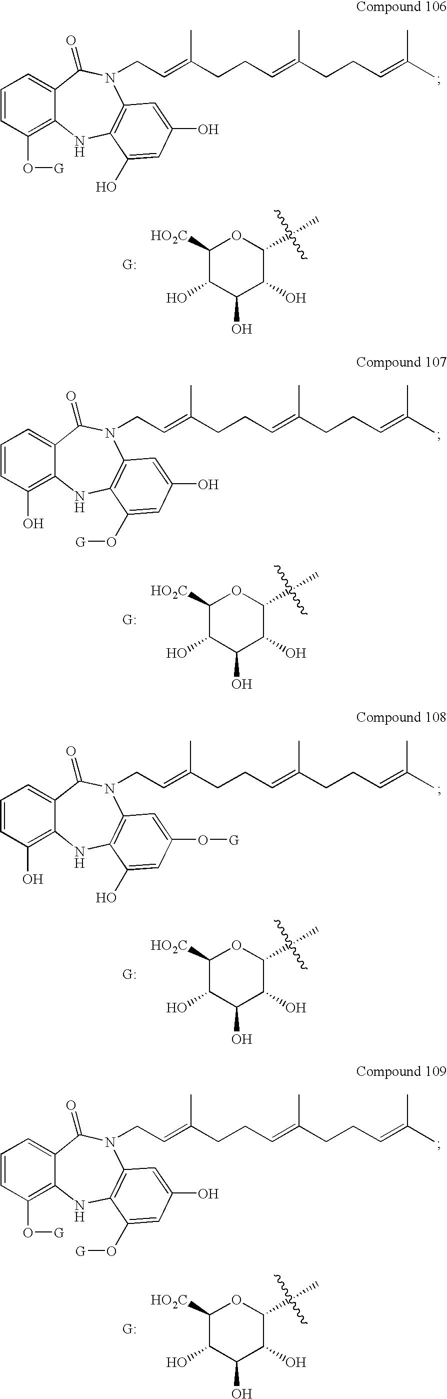 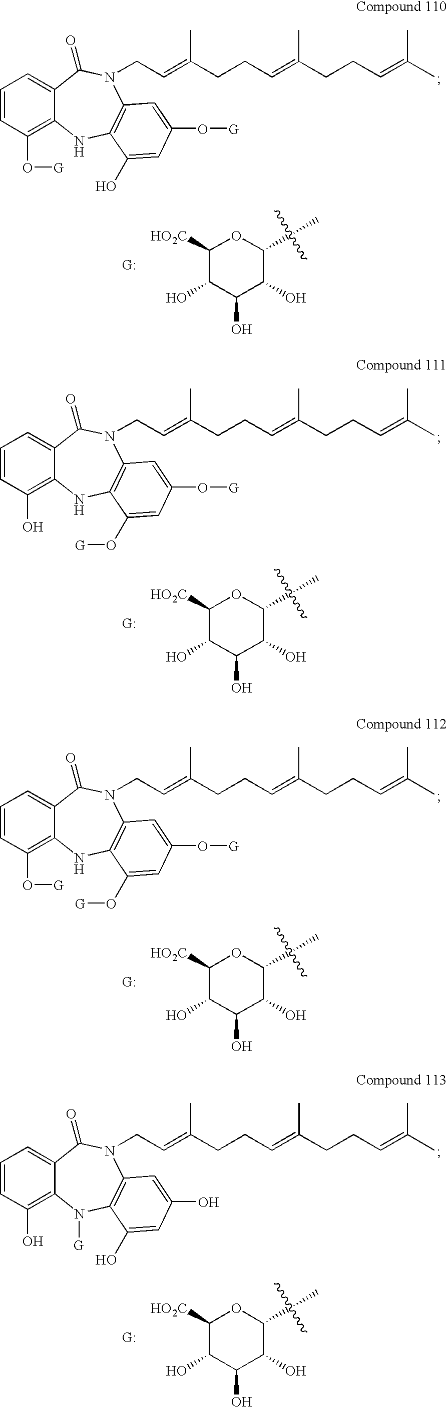 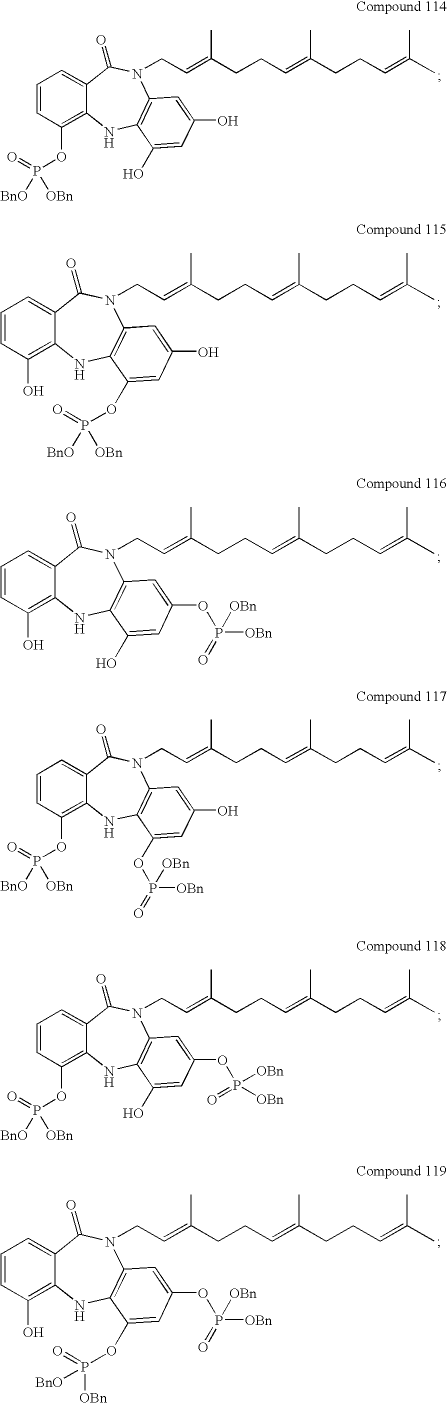 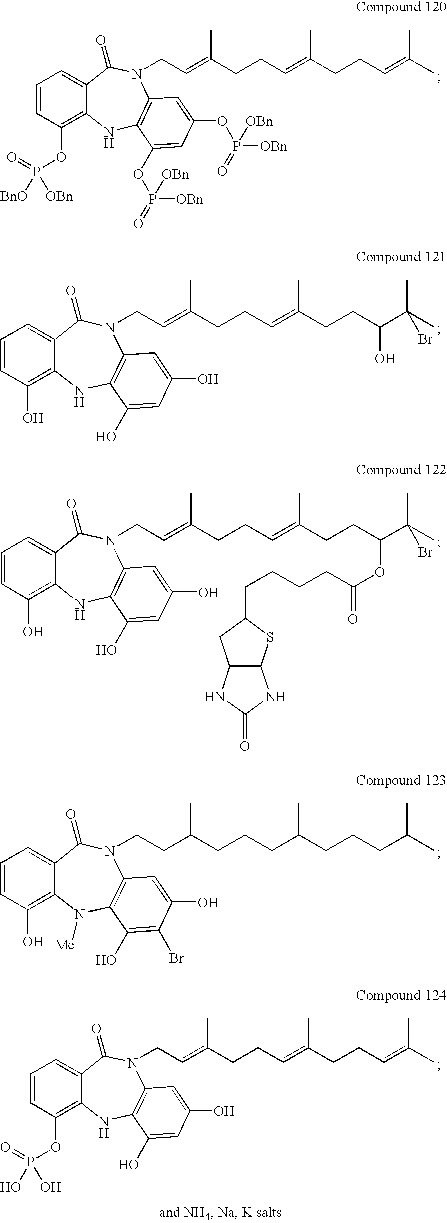 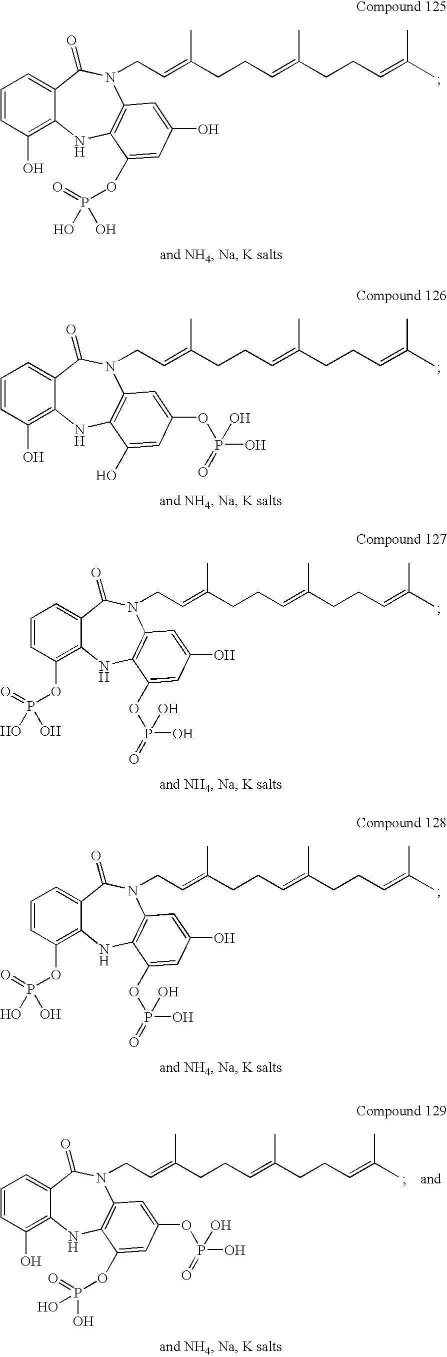 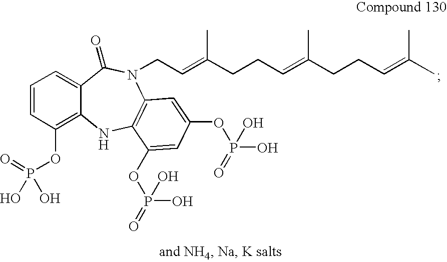
or a pharmaceutically acceptable salt, solvate or prodrug thereof.

3. The pharmaceutical formulation of claim 2, wherein said active ingredient is any one of Compounds 1 to 7, 9 to 11, 14, 17, 18, 46, 63, 64, 67, 77, 78, 80, 82 to 85, 87, 89, 92, 95 to 98, 100 to 103, and 105, or a pharmaceutically acceptable salt, solvate or prodrug thereof.

4. The pharmaceutical formulation of claim 2, wherein said active ingredient is Compound 1: 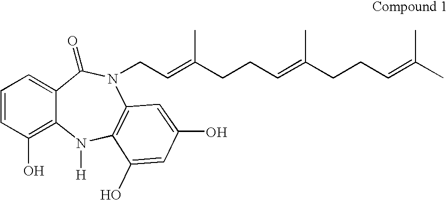
or a pharmaceutically acceptable salt or prodrug thereof.

5. The pharmaceutical formulation of any one of claim 1, wherein said formulation has a weight ratio of surfactant to active ingredient of about 1:1 to about 100:1.

6. The pharmaceutical formulation of claim 5, wherein said formulation has a weight ratio of surfactant to active ingredient of about 2:1 to about 50:1.

7. The pharmaceutical formulation of claim 6, wherein said surfactant is a sorbitan ester, polyoxyethylated castor oil or a phospholipid.

8. The pharmaceutical formulation of claim 7, wherein said surfactant is polysorbate 80.

9. The pharmaceutical formulation of claim 8, wherein said formulation has a weight ratio of polysorbate 80 to active ingredient of about 10:1 to about 25:1.

10. The pharmaceutical formulation of claim 1, wherein said vehicle further comprises a pharmaceutically acceptable solvent.

11. The pharmaceutical formulation of claim 10, wherein said solvent is ethanol and has a weight ratio of ethanol to active ingredient of about 1:1 to about 100:1.

12. The pharmaceutical formulation of claim 11, wherein said weight ratio is about 1:1 to about 50:1.

13. The pharmaceutical formulation of claim 12, wherein said weight ratio is about 1:1 to about 15:1.

14. The pharmaceutical formulation of claim 1, wherein said vehicle further comprises a solubilizer.

15. The pharmaceutical formulation of claim 14, wherein said solubilizer is a hydrophilic polymer.

16. The pharmaceutical formulation of claim 15, wherein said hydrophilic polymer is selected from poly(ethylene glycol) (PEG) or polyvinylpyrrolidone (PVP).

17. The pharmaceutical formulation of claim 14, wherein said formulation has a weight ratio of solubilizer to active ingredient of about 1:1 to about 100:1.

18. The pharmaceutical formulation of claim 15, wherein said formulation has a weight ratio of hydrophilic polymer to active ingredient of about 1:1 to about 50:1.

19. The pharmaceutical formulation of claim 16, wherein said formulation has a weight ratio of hydrophilic polymer to active ingredient of about 1:1 to about 15:1.

20. The pharmaceutical formulation of claim 1, further comprising an antioxidant.

21. The pharmaceutical formulation of claim 20, wherein said antioxidant comprises sodium ascorbate.

22. The pharmaceutical formulation of claim 21, wherein said antioxidant further comprises ascorbic acid.

23. The pharmaceutical formulation of claim 20, wherein said formulation has a ratio of antioxidant to active ingredient of about 1:20 to about 20:1.

24. The pharmaceutical formulation of claim 21, wherein said formulation has a ratio of antioxidant to active ingredient of about 1:5 to about 5:1.

25. The pharmaceutical formulation of claim 21, wherein said formulation has a ratio of antioxidant to active ingredient of about 1:5 to about 2:1.

26. The pharmaceutical composition of claim 1, wherein said formulation further comprises an aqueous component.

27. The pharmaceutical composition of claim 26, wherein said aqueous component is water and has a weight ratio of water to active ingredient of about 1:2 to about 25:1.

28. The pharmaceutical composition of claim 27, wherein the weight ratio of water to active ingredient is about 1:1 to about 10:1.

29. The pharmaceutical composition of claim 26, wherein said aqueous component is selected from 0.9% saline and 5% dextrose and comprises an active ingredient concentration of from about 0.01 to about 50 mg/mL of total volume of formulation.

30. The pharmaceutical formulation of claim 4, wherein said formulation comprises: a surfactant at a weight ratio to active ingredient of about 5:1 to about 30:1; ethanol at a weight ratio to active ingredient of about 1:1 to about 15:1; and a hydrophilic polymer at a weight ratio to active ingredient of about 1:1 to about 15:1.

31. The pharmaceutical formulation of claim 4, wherein said formulation comprises: a surfactant at a weight ratio to active ingredient of about 5:1 to about 30:1; ethanol at a weight ratio to active ingredient of about 1:1 to about 15:1; a hydrophilic polymer at a weight ratio to active ingredient of about 1:1 to about 15:1; and an antioxidant at a weight ratio to active ingredient of about 1:5 to about 5:1.

32. The pharmaceutical formulation of claim 4, wherein said formulation comprises: a surfactant at a weight ratio to active ingredient of about 5:1 to about 30:1; ethanol at a weight ratio to active ingredient of about 1:1 to about 15:1; a hydrophilic polymer at a weight ratio to active ingredient of about 1:1 to about 15:1; an antioxidant at a weight ratio to active ingredient of about 1:5 to about 5:1; and water at a weight ratio to active ingredient of about 1:1 to about 10:1.

33. The pharmaceutical formulation of claim 32, wherein said formulation further comprises an aqueous media, and a concentration in active ingredient ranging from 0.01 to 50 mg/mL of total volume of formulation.

34. A method for preparing a pharmaceutical formulation of claim 1, comprising the step of combining, with mixing, the active ingredient and a surfactant.

35. A method for preparing a pharmaceutical formulation of claim 4, comprising the step of combining, with mixing, the active ingredient and a surfactant.

36. A method of preparing a pharmaceutical formulation of claim 5, comprising the step of combining, with mixing, the active ingredient and a surfactant.

37. A method of preparing a pharmaceutical formulation of claim 11, comprising the step of combining, with mixing, in any order, the active ingredient, a surfactant and ethanol.

38. A method of preparing a pharmaceutical formulation of claim 15, comprising the step of combining, with mixing, in any order, the active ingredient, a surfactant, ethanol and a hydrophilic polymer.

39. A method of preparing a pharmaceutical formulation of claim 20, comprising the step of combining, with mixing, in any order, the active ingredient, a surfactant, ethanol, a hydrophilic polymer and an antioxidant.

40. A method of preparing a pharmaceutical formulation of claim 27, comprising the step of combining, with mixing, in any order, the active ingredient, a surfactant, ethanol, a hydrophilic polymer, an antioxidant and water.

41. A method of preparing a pharmaceutical formulation of claim 32, comprising the steps of combining, with mixing:
a) the active ingredient and ethanol to obtain an ethanolic solution;
b) the antioxidant and water to obtain an aqueous solution;
c) the hydrophilic polymer and the surfactant to obtain a mixture;
d) the ethanolic solution of step (a) and the mixture of step (c); and
e) the aqueous solution of step (b) and the solution of step (d) to produce the pharmaceutical formulation.

42. The method of claim 35, further comprising the step of combining the formulation and an aqueous media selected from 0.9% saline and 5% dextrose.

43. The method of claim 37, further comprising the step of combining the formulation and an aqueous media selected from 0.9% saline and 5% dextrose.

44. The method of claim 41, further comprising the step of combining the formulation and an aqueous media selected from 0.9% saline and 5% dextrose.

45. A method of treating tumor growth in a subject comprising the step of administering a therapeutically effective amount of a formulation of claim 1, to a subject in need thereof.

46. A method of treating tumor growth in a subject comprising the step of administering a therapeutically effective amount of a formulation of claim 4, to a subject in need thereof.

47. A method of treating tumor growth in a subject comprising the step of administering a therapeutically effective amount of a formulation of claim 11, to a subject in need thereof.

48. A method of treating tumor growth in a subject comprising the step of administering a therapeutically effective amount of a formulation of claim 29, to a subject in need thereof.

49. A method of treating tumor growth in a subject comprising the step of administering a therapeutically effective amount of a formulation of claim 30, to a subject in need thereof.

50. A method of treating tumor growth in a subject comprising the step of administering a therapeutically effective amount of a formulation of claim 31, to a subject in need thereof.

51. A method of treating tumor growth in a subject comprising the step of administering a therapeutically effective amount of a formulation of claim 32, to a subject in need thereof.

52. A method of treating tumor growth in a subject comprising the step of administering a therapeutically effective amount of a formulation of claim 33, to a subject in need thereof.

53. The method of claim 52, wherein said formulation is administered by continuous intravenous infusion 24 hours per day for a period of at least 7 days.

54. The method of claim 53, wherein said formulation is administered by continuous intravenous infusion 24 hours per day for a period of 14 days to 28 days.

55. A commercial package comprising a pharmaceutical formulation of claim 1 and instructions for the treatment of a neoplastic condition.

56. A commercial package comprising a pharmaceutical formulation of claim 32 and instructions for the treatment of a neoplastic condition.

57. A commercial package comprising a pharmaceutical formulation of claim 33 and instructions for the treatment of a neoplastic condition.

58. The commercial package of claim 55, wherein the pharmaceutical formulation is in a first sealed vial; and said commercial package further comprising a pre-filled syringe containing an pharmaceutically acceptable aqueous media, suitable for dissolving the content of the first vial.

59. The commercial package of claim 56, wherein the pharmaceutical formulation is in a first sealed vial; and said commercial package further comprising a pre-filled syringe containing an pharmaceutically acceptable aqueous media, suitable for dissolving the content of the first vial.

60. The commercial package of claim 58, further comprising an infusion bag.

61. The commercial package of claim 59, further comprising an infusion bag.
